(19) United States
(12) Patent Application Publication
(10) Pub. No.: US 2024/0409603 A1
(43) Pub. Date: Dec. 12, 2024

(54) MODIFIED STEM CELL COMPOSITIONS AND METHODS FOR USE

(71) Applicant: Jasper Therapeutics, Inc., Redwood City, CA (US)

(72) Inventors: Leopold Daniel D'ESPAUX, San Francisco, CA (US); Song Eun LEE, Redwood City, CA (US); Wendy PANG, Palo Alto, CA (US); Robert SIKORSKI, Woodside, CA (US); Rajiv TIWARI, Alhambra, CA (US)

(21) Appl. No.: 18/702,078

(22) PCT Filed: Oct. 18, 2022
(86) PCT No.: PCT/US2022/078320

§ 371 Date: Apr. 17, 2024

Related U.S. Application Data
(60) Provisional application No. 63/257,010, filed on Oct. 18, 2021.
(60) Provisional application No. 63/272,989, filed on Oct. 28, 2021.

(51) Int. Cl. C07K 14/705; A61K 35/28; A61K 39/395; C12N 5/0789

(52) U.S. Cl. CPC C07K 14/70503; A61K 35/28; A61K 39/3955; C12N 5/0647; C12N 2510/00

(57) ABSTRACT

Provided herein are modified CD117 polypeptides comprising one or more amino acid modifications that inhibit or reduce binding to an anti-CD117 antibody. Also provided are nucleic acids encoding modified CD117 polypeptides, and cells, e.g., hematopoietic stem cells (HSC), in which the CD117 nucleic acid or polypeptide is introduced. The HSC may be used, for example, in hematopoietic cell transplant.

### RELATED APPLICATIONS

This application claims priority to U.S. Provisional Patent Application Ser. No. 63/257,010 filed Oct. 18, 2021 and U.S. Provisional Patent Application Ser. No. 63/272,989 filed Oct. 28, 2021, which are incorporated herein by reference in their entireties.

### SEQUENCE LISTING

This application is being filed electronically via Patent Center and includes an electronically submitted sequence listing in .xml format. The .xml file contains a sequence listing entitled JATH_003_01WO_SeqList_ST26.xml created on Oct. 18, 2022 and having a size of 71,381 bytes. The sequence listing contained in this.xml file is part of the specification and is incorporated herein by reference in its entirety.

### FIELD OF THE INVENTION

The present disclosure relates to modified hematopoietic stem cells comprising a modified CD117 that is not bound by an anti-CD117 antibody, and their use for hematopoietic stem cell transplantation.

### BACKGROUND

Hematopoietic cell transplantation (HCT) generally involves the intravenous infusion of autologous or allogeneic donor hematopoietic stem cells (HSCs) or hematopoietic stem and progenitor cells (HPSCs) obtained from bone marrow, peripheral blood, or umbilical cord blood into a subject whose bone marrow or immune system is damaged or defective. HCT may be performed as part of therapy to treat a number of disorders, including cancers, such as leukemias, and congenital immunodeficiency disorders.

HCT is usually accompanied by a preparative or conditioning regimen to clear bone-marrow niches of endogenous HSCs, in order for donor HSCs to engraft. Current conditioning regimens may include treatment with DNA damaging radiation and/or chemotherapy, which can have toxic effects that limit the use of HCT. More recently, a non-genotoxic approach of targeting and depleting HSC has been developed, which uses an antibody that binds human CD117 (c-Kit), a receptor tyrosine kinase expressed on the surface of HSC and progenitor cells (HSPC). Treatment with the anti-CD117 antibody has been shown to suppress human hematopoiesis in vitro, deplete human HSC in mice xenografted with human cells, and safely deplete HSC of non-human primates (Agarwal, R. et al., Blood (2019) 134 (Supplement_1): 800.

Nonetheless, there remains a need in the art for improved compositions and methods for HCT, including conditioning methods with reduced toxicity. The present disclosure addresses this need.

### BRIEF SUMMARY OF THE INVENTION

The present disclosure provides inter alia novel modified CD117 polypeptides and related compositions and methods of use thereof in hematopoietic stem cell transplant. In particular embodiments, the modified CD117 polypeptides are not bound by an anti-CD117 antibody used for HCT conditioning, and are capable of signaling in HSCs and/or HSPCs in response to stem cell factor (SCF) binding. In certain embodiments, the modified CD117 polypeptides provide for SCF-mediated signaling and/or kinase activity when expressed in cells, e.g., HSCs and/or HSPCs. Accordingly, in particular embodiments, when expressed in HSCs and/or HSPCs, the modified CD117 polypeptides allow CD117 signaling in the presence of antibodies that block SCF binding to wild type CD117. In certain embodiments, the anti-CD117 antibody disrupts or blocks binding of SCF to wild type CD117.

In one aspect, the disclosure provides a modified CD117 polypeptide comprising one or more amino acid modifications as compared to a wild type CD117 polypeptide, e.g., one or more amino acid substitutions, insertions, or deletions. In certain embodiments, the modified CD117polypeptide comprises one or more amino acid substitutions, e.g., at one or more of the following amino acids present in wild type human CD117: E73, D121, R122, S123, Y125, K203, Y259, S261, W262, Y269, or R271, or at an amino acid residue located within 2, within 3, within 4, within 5, within 6, within 7 within 8 within 8 within 10, within 11 or within 12 amino acids of any of E73, D121, R122, S123, Y125, K203, Y259, S261, W262, Y269, or R271, e.g., either N-terminal or C-terminal of any of these residues. In particular embodiments, the one or more amino acid modifications is located within surface exposed amino acid residues of the extracellular domain of the wild type CD117 polypeptide. In particular embodiments, the modified CD117 polypeptide has at least 90%, at least 95%, at least 98%, or at least 99% sequence homology to the wild type CD117 polypeptide, or a functional fragment thereof. In certain embodiments, the wild type CD117 polypeptide is a wild type human CD117 polypeptide, optionally having one of the following amino acid sequences:

(SEQ ID NO: 1)
MRGARGAWDFLCVLLLLLRVQTGSSQPSVSPGEPSPPSIHPGKSDLIVRVGDEIRLLCTDP
GFVKWTFEILDETNENKQNEWITEKAEATNTGKYTCTNKHGLSNSIYVFVRDPAKLFLV
DRSLYGKEDNDTLVRCPLTDPEVTNYSLKGCQGKPLPKDLRFIPDPKAGIMIKSVKRAY
HRLCLHCSVDQEGKSVLSEKFILKVRPAFKAVPVVSVSKASYLLREGEEFTVTCTIKDVS
SSVYSTWKRENSQTKLQEKYNSWHHGDFNYERQATLTISSARVNDSGVFMCYANNTFG
SANVTTTLEVVDKGFINIFPMINTTVFVNDGENVDLIVEYEAFPKPEHQQWIYMNRTFTD
KWEDYPKSENESNIRYVSELHLTRLKGTEGGTYTFLVSNSDVNAAIAFNVYVNTKPEILT
YDRLVNGMLQCVAAGFPEPTIDWYFCPGTEQRCSASVLPVDVQTLNSSGPPFGKLVVQS
SIDSSAFKHNGTVECKAYNDVGKTSAYFNFAFKGNNKEQIHPHTLFTPLLIGFVIVAGM
MCIIVMILTYKYLQKPMYEVQWKVVEEINGNNYVYIDPTQLPYDHKWEFPRNRLSFGK
TLGAGAFGKVVEATAYGLIKSDAAMTVAVKMLKPSAHLTEREALMSELKVLSYLGNH
MNIVNLLGACTIGGPTLVITEYCCYGDLLNFLRRKRDSFICSKQEDHAEAALYKNLLHSK
ESSCSDSTNEYMDMKPGVSYVVPTKADKRRSVRIGSYIERDVTPAIMEDDELALDLEDL
LSFSYQVAKGMAFLASKNCIHRDLAARNILLTHGRITKICDFGLARDIKNDSNYVVKGN
ARLPVKWMAPESIFNCVYTFESDVWSYGIFLWELFSLGSSPYPGMPVDSKFYKMIKEGF
RMLSPEHAPAEMYDIMKTCWDADPLKRPTFKQIVQLIEKQISESTNHIYSNLANCSPNRQ
KPVVDHSVRINSVGSTASSSQPLLVHDDV;
or
(SEQ ID NO: 2)
MRGARGAWDFLCVLLLLLRVQTGSSQPSVSPGEPSPPSIHPGKSDLIVRVGDEIRLLCTDP
GFVKWTFEILDETNENKQNEWITEKAEATNTGKYTCTNKHGLSNSIYVFVRDPAKLFLV
DRSLYGKEDNDTLVRCPLTDPEVTNYSLKGCQGKPLPKDLRFIPDPKAGIMIKSVKRAY
HRLCLHCSVDQEGKSVLSEKFILKVRPAFKAVPVVSVSKASYLLREGEEFTVTCTIKDVS
SSVYSTWKRENSQTKLQEKYNSWHHGDFNYERQATLTISSARVNDSGVFMCYANNTFG
SANVTTTLEVVDKGFINIFPMINTTVFVNDGENVDLIVEYEAFPKPEHQQWIYMNRTFTD
KWEDYPKSENESNIRYVSELHLTRLKGTEGGTYTFLVSNSDVNAAIAFNVYVNTKPEILT
YDRLVNGMLQCVAAGFPEPTIDWYFCPGTEQRCSASVLPVDVQTLNSSGPPFGKLVVQS
SIDSSAFKHNGTVECKAYNDVGKTSAYFNFAFKEQIHPHTLFTPLLIGFVIVAGMMCIIV
MILTYKYLQKPMYEVQWKVVEEINGNNYVYIDPTQLPYDHKWEFPRNRLSFGKTLGAG
AFGKVVEATAYGLIKSDAAMTVAVKMLKPSAHLTEREALMSELKVLSYLGNHMNIVN
LLGACTIGGPTLVITEYCCYGDLLNFLRRKRDSFICSKQEDHAEAALYKNLLHSKESSCS
DSTNEYMDMKPGVSYVVPTKADKRRSVRIGSYIERDVTPAIMEDDELALDLEDLLSFSY
QVAKGMAFLASKNCIHRDLAARNILLTHGRITKICDFGLARDIKNDSNYVVKGNARLPV
KWMAPESIFNCVYTFESDVWSYGIFLWELFSLGSSPYPGMPVDSKFYKMIKEGFRMLSP
EHAPAEMYDIMKTCWDADPLKRPTFKQIVQLIEKQISESTNHIYSNLANCSPNRQKPVVD
HSVRINSVGSTASSSQPLLVHDDV.

In particular embodiments, the modified CD117 polypeptide substantially retains CD117 signaling and/or kinase activity as compared to the wild type CD117 polypeptide. In particular embodiments, the modified CD117 polypeptide substantially retains CD117 signaling and/or kinase activity in response to SCF binding, as compared to the wild type CD117 polypeptide. In particular embodiments, at least 30%, at least 40%, at least 50%, at least 60%, at least 70%, at least 80%, at least 90%, or at least 95% of CD117 signaling and/or kinase activity in response to SCF binding is retained in cells, e.g., HSCs and/or HSPCs, expressing the modified CD117 polypeptide.

In particular embodiments, the one or more amino acid modifications do not substantially inhibit or reduce CD117 signaling or cell proliferation or cell viability, optionally in response to stem cell factor (SCF) binding, by the modified CD117 polypeptide expressed in cells, e.g., HSCs and/or HSPCs, as compared to the wild type CD117 polypeptide.

In particular embodiments, the one or more amino acid modifications inhibit or reduce binding of an anti-CD117 antibody to the modified CD117 polypeptide expressed in cells e.g., HSCs and/or HSPCs, as compared to the wild type CD117 polypeptide. In particular embodiments, binding is inhibited by at least at least 30%, at least 40%, at least 50%, at least 60%, at least 70%, at least 80%, at least 90%, or at least 95%. In particular embodiments, the anti-CD117 antibody comprises the six CDRs present in any one of JSP191, AB85, CDX-0159, or FSI-174. In particular embodiments, the anti-CD117 antibody is any one of JSP191, AB85, CDX-0159, or FSI-174.

In particular embodiments, the one or more amino acid modifications do not substantially inhibit or reduce binding of stem cell factor (SCF) to the modified CD117 polypeptide expressed in cells e.g., HSCs and/or HSPCs, as compared to the wild type CD117 polypeptide. In particular embodiments, binding is inhibited by less than 10%, less than 20%, less than 30$, less than 40%, less than 50%, or less than 60%.

In a related embodiment, the disclosure provides a nucleic acid encoding the modified CD117 polypeptide. In some embodiments, the nucleic acid sequence comprises any one of SEQ ID NOS: 5-7, or a sequence having at least 50%, at least 55%, at least 60%, at least 70%, at least 75%, at least 80%, at least 85%, at least 90%, at least 95%, at least 96%, at least 97%, at least 98%, or at least 99%, sequence identity to any one of SEQ ID NOS: 5-7. 19. In certain embodiments, the nucleic acid, optionally an mRNA or modified mRNA comprises:
a) a sequence of any one of SEQ ID NOS: 5-7, optionally further comprising a poly A tail, optionally an A70 poly A tail;
b) a sequence having at least 50%, at least 55%, at least 60%, at least 70%, at least 75%, at least 80%, at least 85%, at least 90%, at least 95%, at least 96%, at least 97%, at least 98%, or at least 99%, sequence identity to any one of SEQ ID NOS: 5-7, optionally further comprising a poly A tail, optionally an A70 poly A tail;
c) a coding sequence of any one of SEQ ID NOS: 5-7;
d) a sequence having at least 50%, at least 55%, at least 60%, at least 70%, at least 75%, at least 80%, at least 85%, at least 90%, at least 95%, at least 96%, at least 97%, at least 98%, or at least 99%, sequence identity to a coding sequence of any one of SEQ ID NOS: 5-7;
e) a sequence comprising the Cap sequence, Kozak sequence, and coding sequence of any one of SEQ ID NOS: 5-7, optionally further comprising a poly A tail, optionally an A70 poly A tail; or
f) a sequence having at least 50%, at least 55%, at least 60%, at least 70%, at least 75%, at least 80%, at least 85%, at least 90%, at least 95%, at least 96%, at least 97%, at least 98%, or at least 99%, sequence identity to a sequence comprising the Cap sequence, Kozak sequence, and coding sequence of any one of SEQ ID NOS: 5-7, optionally further comprising a poly A tail, optionally an A70 poly A tail. In particular embodiments, the nucleic acid comprises RNA, DNA, or a combination thereof. In some embodiments, the nucleic acid comprises a modified mRNA. In some embodiments, the nucleic acid is associated with one or more lipids, optionally wherein the nucleic acid is present within a lipid nucleic acid particle, a lipid nanoparticle, or a liposome.

In a further related embodiment, the disclosure provides a vector comprising the nucleic acid encoding the modified CD117 polypeptide. In certain embodiments, the vector is an expression vector, e.g., an AAV vector or a lentiviral vector. In particular embodiments, the vector is capable of transducing hematopoietic stem cells.

In some embodiments, the disclosure provides mRNAs encoding a wild type or modified CD117 polypeptide. In some embodiments, the mRNA encodes a E73A mutant CD117 polypeptide. In some embodiments, the CD117 mRNA is introduced into an HSC and/or HSPC. In some embodiments the cell is CD34+, and in some embodiments, the cell is CD34+/CD90+, CD34+/CD38−, CD34+/CD38−/CD90+, or CD34+/CD133+.

In some embodiments, the disclosure provides a modified cell, e.g., HSC and/or HSPC, comprising the modified CD117 polypeptide and/or the nucleic acid encoding the modified CD117 polypeptide. In particular embodiments, the modified cell expresses both the modified CD117 polypeptide and wild type CD117 polypeptide. In particular embodiments, the modified cell expresses the modified CD117 polypeptide but not the wild type CD117 polypeptide. In certain embodiments, the modified cell was transduced with the vector. In certain embodiments, the endogenous gene encoding the wild type CD117 polypeptide is genetically modified to encode the modified CD117 polypeptide, e.g., by gene editing, such as CRISPR-Cas9 gene editing, TALEN gene editing, zinc finger gene editing, or homing endonuclease or meganuclease gene editing. In certain embodiments, the gene editing is performed using a base editing method.

In certain embodiments, the cell is a stem cell, e.g., a hematopoietic stem cell (HSC) or a hematopoietic stem and progenitor cell (HSPC). In particular embodiments, the cell is CD34+, and in some embodiments, the cell is CD34+/CD90+, CD34+/CD38−, CD34+/CD38−/CD90+, or CD34+/CD133+. In some embodiments, the cell is a human cell. In some embodiments, the cell was obtained from a mammalian donor. In certain embodiments, the mammalian donor is a subject in need of a hematopoietic stem cell transplant (autologous donor), wherein in other embodiments, the mammalian donor is not the subject in need of the hematopoietic stem cell transplant (allogeneic donor). In certain embodiments, the cell expresses the modified CD117 polypeptide, optionally wherein the modified cell expresses the modified CD117 polypeptide transiently or constitutively. In particular embodiments, the modified CD117 polypeptide is expressed on the cell surface or in the cell membrane, and in certain embodiments, the cell is capable of proliferating in the presence of an anti-CD117 antibody. In certain embodiments, the anti-CD117 antibody is capable of inhibiting proliferation and/or survival of a cell expressing only the wild-type CD117. In some embodiments, the anti-CD117 antibody induces apoptosis or death of a cell expressing only the wild-type CD117. In certain embodiments, contact with or the presence of the anti-CD117 antibody results in less than 50%, less than 40%, less than 30%, less than 20%, or less than 10% as much cell death in cells expressing the modified CD117 polypeptide as compared to in cells expressing only the wild-type CD117 polypeptide. In some embodiments, the anti-CD117 antibody is selected from the group consisting of: JSP191, CDX-0159, AB85, and FSI-174.

In a further related embodiment, the disclosure provides a pharmaceutical composition comprising the modified cells, e.g., HSCs and/or HSPCs, comprising the nucleic acid encoding the modified CD117 polypeptide, and a pharmaceutically acceptable excipient, carrier, or diluent. In some embodiments, the pharmaceutical composition further comprises an anti-CD117 antibody. In certain embodiments, the pharmaceutical composition further comprises one or more anti-CD47, anti-CD40L, anti-CD122, anti-CD4, and/or anti-CD8 antibody.

In a related aspect, the disclosure includes a method of modifying a cell, e.g., an HSC and/or HSPC, comprising introducing a nucleic acid or vector encoding a modified CD117 polypeptide into the cell, optionally wherein the cell is transiently modified or permanently modified, and optionally wherein the method is for preparing modified cells for hematopoietic cell transplantation (HCT) into a mammalian subject. In certain embodiments, the nucleic acid or vector is introduced into the cell by transfection, transduction, infection, electroporation, or nanopore technology. In certain embodiments, the cell is modified by gene editing.

In another aspect, the disclosure includes a method of treating a mammalian subject in need thereof, comprising administering to the subject a pharmaceutical composition comprising modified cells, e.g., HSCs, comprising the nucleic acid encoding the modified CD117 polypeptide and/or the modified CD117 polypeptide. In some embodiments, the method further comprises administering to the subject a conditioning regimen to facilitate or increase engraftment of the modified cells, or deplete endogenous, wild-type HSCs, wherein the conditioning regimen is administered prior to and/or concurrent with and/or following the administering of the pharmaceutical composition. In some embodiments, the conditioning regimen comprises or consists of an anti-CD117 antibody, optionally JSP191. In some embodiments, the conditioning regimen comprises one or more of: chemotherapy (optionally a nucleoside analog and/or an alkylating agent), monoclonal antibody therapy, and radiation, optionally radiation to the entire body. In particular embodiments, the conditioning regimen is milder than would be used if the subject was being administered hematopoietic stem cells that did not comprise the modified CD117 polypeptide. In some embodiments, the subject is not administered a conditioning regimen to facilitate or increase engraftment of the modified cells, prior to or concurrent with the administering of the pharmaceutical composition, or the conditioning regimen only comprises the anti-CD117 antibody. In particular embodiments, the method results in reduced toxicity, reduced morbidity, or reduced graft-versus-host disease, as compared to a method wherein a subject is administered hematopoietic stem cells that do not comprise the modified CD117 polypeptide in combination with a conditioning regimen, e.g., a reduction of at least 10%, at least 20%, at least 30%, at least 40%, or at least 50% in toxicity, morbidity, and/or graft-versus-host disease.

In particular embodiments, the method is used to treat a disease or disorder selected from the group consisting of: a cancer, a cardiac disorder, a neural disorder, an autoimmune disease, an immunodeficiency, a metabolic disorder, and a genetic disorder. In certain embodiments, the cancer is a solid tissue cancer or a blood cancer, e.g., a leukemia, a lymphoma, or a myelodysplastic syndrome, such as acute myeloid leukemia (AML). In certain embodiments, the immunodeficiency is severe combined immunodeficiency (SCID). In certain embodiments, the genetic disorder is sickle cell disease or Fanconi anemia. In some embodiments, the methods further comprise administering to the subject another therapeutic agent for treatment of the disease or disorder.

### BRIEF DESCRIPTION OF THE DRAWINGS

FIGS. 1A-1C are graphs showing binding of JSP191 (FIG. 1A) or AB85 (FIG. 1B (whole view) and FIG. 1C (zoomed in view)) to clones of an alanine scanning library. Mean binding value is plotted as a function of expression (represented by clone reactivity) for each clone. Critical clones that do not bind JSP191 or AB85 are represented in black (lower right quadrant); the remaining mutant clones are represented in grey.

FIG. 2 is a table showing the results of alanine scanning mutagenesis of CD117 and binding of JSP191Fab, AB85Fab and 104D2 Mab. Mean binding reactivity (and range) of each to clones comprising the indicated point mutations in CD117 is shown as % binding relative to the binding to wild type CD117. Critical residues for JSP191 and AB8 5binding are shaded.

FIGS. 3A-3B show crystal structures of JSP191 Fab (FIG. 3A) and AB85 Fab (FIG. 3B) in contact with CD117. Critical residues of CD117 are represented as dark spheres in the crystal structure of the Abs (Yuzawa et al., 2007).

FIG. 4 is a table listing the critical residues whose mutation gave the lowest reactivities with the JSP191 or AB85 antibody.

FIG. 5 is a schematic of CD117 (cKit) interaction with stem cell factor (SCF) to regulate HSC survival, self-renewal, and differentiation.

FIG. 6 is a table of DNA template sequences used to produce the CD117 mRNAs provided herein, including the name, size, and description of the mRNA sequences (which can be found in the attached sequence listing and are denoted as SEQ ID NOS: 5-7). The sequences of SEQ ID NOS: 5-7 include a T7 promoter compatible with CleanCapAG, CleanCapAG, Kozak sequence, an HBA1 5′ UTR, coding sequence, a TAATGA double stop codon, an HBB1 3′ UTR, but do not include the poly-adenosine tail of 70 nucleotides, which is also present in the DNA templates. The corresponding mRNA sequences comprise the same sequence absent the T7 promoter, and it is understood that for all provided DNA sequences, the corresponding mRNA sequences comprise Us instead of Ts (which may be further modified).

FIG. 7 is a graph of CD117 expression from different CD117 mRNAs or controls 20 hours after electroporation.

FIGS. 8A and 8B are graphs of cell viability 20 hours post electroporation with the indicated mRNAs or controls. FIG. 8A is a graph of live cell numbers; FIG. 8B is a graph of percent viability relative to cell death.

FIG. 9 is a graph of CD17 expression from different CD117 mRNAs or controls 3 hours after electroporation.

FIG. 10 is a graph of cell count versus CD117 expression in human CD34+ cells expressing mock, control (null), wild type, and E73A CD117 mRNAs.

FIGS. 11A-11C are graphs of CD117 expression in human CD34+ cells over time showing initially two distinct levels of wild type and E73A CD117 expression which resolve by 20 hours after electroporation. FIG. 11A shows cell count versus CD117 expression 3 hours after electroporation; FIG. 11B shows cell count versus CD117 expression 20 hours after electroporation; and FIG. 11C shows cell count versus CD117 expression 48 hours after electroporation.

FIG. 12 is a graph of the level of CD117 (cKit) expression for mock electroporation (Mock EP), null control (Ctrl), wild type (WT), and E73A CD117 expressing cells.

FIGS. 13A and 13B are graphs showing that expression of CD117 E73A mutants in Ba/F3 cells leads to JSP191 resistance. Cells in these experiments were transfected with lentiviral constructs encoding wild type and E73 CD117 proteins. FIG. 13A shows growth of Ba/F3 cells based on wild type and E73A CD117 expression in the presence of human stem cell factor (hSCF). FIG. 13B shows growth of Ba/F3 cells based on wild type and E73A CD117 expression in the presence of the JSP191 antibody.

FIGS. 14A-14B are graphs showing the effects of CD117 expression on cell growth in the presence of stem cell factor (SCF) and the JSP91 antibody. FIG. 14A shows the growth of human CD34+ cells transfected without mRNA. FIG. 14B shows the growth of cells transfected with wild type CD117 mRNA (as encoded by SEQ ID NO: 54).

### DETAILED DESCRIPTION OF THE INVENTION

Hematopoietic stem cell transplantation (HCT) can be curative therapy for many diseases, based on the principle that healthy hematopoietic stem cells (HSCs) and/or hematopoietic stem and progenitor cells (HSPCs) replace abnormal HSCs. However, HCT is not widely used due to the toxicities associated with the current practices of this procedure. The deleterious effects of HCT can include substantial tissue injury and even mortality from the use of chemotherapy and/or radiation prior to transplant (which are needed to prepare recipients to accept donor or autologous gene-corrected cells) and graft-vs-host disease (GVHD) caused by donor lymphocytes that are contained within allogeneic hematopoietic grafts. Despite the known complications caused by chemotherapy and/or radiation, and the infusion of donor lymphocytes in the allograft, these modalities are incorporated into HCTs, because they facilitate the engraftment of donor HSCs. Furthermore, HCTs can fail because donor HSC fail to engraft and/or fail to persist following the HCT procedure.

Certain HCT procedures include conditioning a patient prior to HCT by treatment with an anti-CD117 antibody that inhibits stem cell factor (SCF) from binding to CD117 on the surface of a patient's endogenous HSCs, which depletes endogenous HSCs prior to transplant of HSCs and/or HSPCs into the patient. However, this typically requires a significant washout period of about a week or more following administration of the anti-CD117 antibody, so there are few remaining antibodies that would deplete the transplanted HSCs and/or HSPCs.

The present disclosure provides compositions and methods that augment the ability of donor or autologous gene-corrected HSCs and/or HSPCs to engraft and/or persist in recipients, thereby increasing the likelihood of success of an HCT procedure, and reducing the toxicities associated with HCT. In particular, the disclosure provides modified HSCs and/or HSPCs for transplant that comprise a modified CD117 polypeptide that has reduced binding to anti-CD117 antibodies used for conditioning prior to HCT. Accordingly, the modified cells can be transplanted into the subject in the presence of the anti-CD117 antibodies without being subject to depletion, thus providing an improved method of conditioning a patient for HCT and potentially allowing a reduced (or no) washout period and/or other advantages. The HCT methods provided herein may also result in a reduction in the need for intensive chemotherapy, radiation, and/or donor lymphocytes or other cells used to facilitate HSC engraftment, thereby reducing the toxicity of HCT. Compositions and methods disclosed herein may be used to treat all disorders for which blood stem cell transplantation is indicated.

In certain embodiments, the disclosure provides for compositions and methods for the ex vivo introduction of CD117 variants and mutants (modified CD117), by RNA-based and/or DNA-based methods, into HSCs and/or HSPCs, including but not limited to CD34+ cells or subsets of CD34+ cells, such that the HSCs and/or HSPCs are able to be successfully transplanted into recipients. The modified CD117 may be expressed transiently or constitutively in the modified HSCs and/or HSPCs. For example, a nucleic acid encoding a modified CD117 may be transiently introduced into HSCs/and/or HSPCs prior to transplant, where it expresses the modified CD117. Alternatively, HSCs and/or HSPCs may be altered by gene editing methodologies, such as the use of CRISPR/Cas9, TALENS, zing finger nucleases, or homing endonucleases or meganucleases. Thus, the modified CD117 may be expressed in additional to the endogenous wild type CD117, or it may replace the endogenous CD117. Transplantation of these modified HSCs may be done after or in combination with conditioning therapies, including treatment with antibodies (such as anti-CD117 antibodies). These HSCs may be transplanted alone or in combination with other cells.

It is to be understood that this invention is not limited to the particular methodology, products, apparatus and factors described, as such methods, apparatus and formulations may, of course, vary. It is also to be understood that the terminology used herein is for the purpose of describing particular embodiments only, and it is not intended to limit the scope of the present invention which will be limited only by appended claims.

It must be noted that as used herein and in the appended claims, the singular forms “a,” “and,” and “the” include plural referents unless the context clearly dictates otherwise. Thus, for example, reference to “a drug candidate” refers to one or mixtures of such candidates, and reference to “the method” includes reference to equivalent steps and methods known to those skilled in the art, and so forth.

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

As used herein, “antibody” includes reference to an immunoglobulin molecule immunologically reactive with a particular antigen, and includes both polyclonal and monoclonal antibodies. The term also includes genetically engineered forms such as humanized antibodies, chimeric antibodies (e.g., humanized murine antibodies) and heteroconjugate antibodies. The term “antibody” also includes antigen binding forms of antibodies, including fragments with antigen-binding capability (e.g., Fab′, F(ab′)₂, Fab, Fv and rIgG. The term also refers to recombinant single chain Fv fragments (scFv). The term antibody also includes bivalent or bispecific molecules, diabodies, triabodies, and tetrabodies.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin.

The assignment of amino acids to each VL and VH domain (and the CDRs therein) is in accordance with any conventional definition of CDRs. Conventional definitions include: the Kabat definition (Kabat, Sequences of Proteins of Immunological Interest (National Institutes of Health, Bethesda, Md., 1987 and 1991); the Chothia definition (Chothia & Lesk, J. Mol. Biol. 196:901-917, 1987; Chothia et al., Nature 342:878-883, 1989); a composite of Chothia Kabat CDR in which CDR-H1 is a composite of Chothia and Kabat CDRs; the AbM definition used by Oxford Molecular's antibody modelling software; and, the contact definition of Martin et al. (world wide web bioinfo.org.uk/abs). Kabat provides a widely used numbering convention (Kabat numbering) in which corresponding residues between different heavy chains or between different light chains are assigned the same number. Unless otherwise specified numbering of positions within the variable regions of antibodies is Kabat numbering. When an antibody is said to comprise CDRs by a certain definition of CDRs (e.g., Kabat) that definition specifies the minimum number of CDR residues present in the antibody (i.e., the Kabat CDRs). It does not exclude that other residues falling within another conventional CDR definition but outside the specified definition are also present. For example, an antibody comprising CDRs defined by Kabat includes among other possibilities, an antibody in which the CDRs contain Kabat CDR residues and no other CDR residues, and an antibody in which CDR HI is a composite Chothia-Kabat CDR HI and other CDRs contain Kabat CDR residues and no additional CDR residues based on other definitions.

The term “polynucleotide” refers to a polymeric form of nucleotides of any length, including deoxyribonucleotides or ribonucleotides, or analogs or mixtures thereof. A polynucleotide may comprise modified nucleotides, such as methylated nucleotides and nucleotide or nucleoside analogs, and may be interrupted by non-nucleotide components. If present, modifications to the nucleotide structure may be imparted before or after assembly of the polymer. The term polynucleotide, as used herein, includes, but is not limited to, double- and single-stranded molecules, and mixtures thereof. Unless otherwise specified or required, any embodiment of the invention described herein that is a polynucleotide encompasses both the double-stranded form and each of two complementary single-stranded forms known or predicted to make up the double-stranded form, whether as RNA or DNA, or a mixture thereof.

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

A polynucleotide or polypeptide has a certain percent “sequence identity” to another polynucleotide or polypeptide, meaning that, when aligned, that percentage of bases or amino acids are the same when comparing the two sequences. Sequence similarity can be determined in a number of different manners. To determine sequence identity, sequences can be aligned using the methods and computer programs, including BLAST, available over the worldwide web at ncbi.nlm.nih.gov/BLAST/. Another alignment algorithm is FASTA, available in the Genetics Computing Group (GCG) package, from Madison, Wis., USA, a wholly owned subsidiary of Oxford Molecular Group, Inc. Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Of particular interest are alignment programs that permit gaps in the sequence. The Smith-Waterman is one type of algorithm that permits gaps in sequence alignments. See Meth. Mol. Biol. 70: 173-187 (1997). Also, the GAP program using the Needleman and Wunsch alignment method can be utilized to align sequences. See J. Mol. Biol. 48: 443-453 (1970).

Of interest is the BestFit program using the local homology algorithm of Smith and Waterman (Advances in Applied Mathematics 2: 482-489 (1981) to determine sequence identity. The gap generation penalty will generally range from 1 to 5, usually 2 to 4 and in many embodiments will be 3. The gap extension penalty will generally range from about 0.01 to 0.20 and in many instances will be 0.10. The program has default parameters determined by the sequences inputted to be compared. Preferably, the sequence identity is determined using the default parameters determined by the program. This program is available also from Genetics Computing Group (GCG) package, from Madison, Wis., USA.

Another program of interest is the FastDB algorithm. FastDB is described in Current Methods in Sequence Comparison and Analysis, Macromolecule Sequencing and Synthesis, Selected Methods and Applications, pp. 127-149, 1988, Alan R. Liss, Inc. Percent sequence identity is calculated by FastDB based upon the following parameters: Mismatch Penalty: 1.00; Gap Penalty: 1.00; Gap Size Penalty: 0.33; and Joining Penalty: 30.0.

A “vector” as used herein refers to a macromolecule or association of macromolecules that comprises or associates with a polynucleotide and which can be used to mediate delivery of the polynucleotide to a cell. Illustrative vectors include, for example, plasmids, viral vectors, liposomes, and other gene delivery vehicles.

An “expression vector” as used herein encompasses a vector, e.g. plasmid, minicircle, viral vector, liposome, and the like as discussed herein or as known in the art, comprising a polynucleotide which encodes a gene product of interest, and is used for effecting the expression of a gene product in an intended target cell. An expression vector also comprises control elements operatively linked to the encoding region to facilitate expression of the gene product in the target. The combination of control elements, e.g., promoters, enhancers, UTRs, miRNA targeting sequences, etc., and a gene or genes to which they are operably linked for expression is sometimes referred to as an “expression cassette.” Many such control elements are known and available in the art or can be readily constructed from components that are available in the art.

A “promoter” as used herein encompasses a DNA sequence that directs the binding of RNA polymerase and thereby promotes RNA synthesis, i.e., a minimal sequence sufficient to direct transcription. Promoters and corresponding protein or polypeptide expression may be ubiquitous, meaning strongly active in a wide range of cells, tissues and species, or it may be cell-type specific, tissue-specific, or species specific. Promoters may be “constitutive,” meaning continually active, or “inducible,” meaning the promoter can be activated or deactivated by the presence or absence of biotic or abiotic factors.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

The term “native” or “wild-type” as used herein refers to a nucleotide sequence, e.g. gene, or gene product, e.g. RNA or polypeptide, that is present in a wild-type cell, tissue, organ or organism. The term “variant” as used herein refers to a mutant of a reference polynucleotide or polypeptide sequence, for example a native polynucleotide or polypeptide sequence, i.e., having less than 100% sequence identity with the reference polynucleotide or polypeptide sequence. Put another way, a variant comprises at least one amino acid difference (e.g., amino acid substitution, amino acid insertion, amino acid deletion) relative to a reference polynucleotide sequence, e.g., a native polynucleotide or polypeptide sequence. For example, a variant may be a polynucleotide having a sequence identity of 50% or more, 60% or more, or 70% or more with a full length native polynucleotide sequence, e.g., an identity of 75% or 80% or more, such as 85%, 90%, or 95% or more, for example, 98% or 99% identity with the full length native polynucleotide sequence. As another example, a variant may be a polypeptide having a sequence identity of 70% or more with a full length native polypeptide sequence, e.g., an identity of 75% or 80% or more, such as 85%, 90%, or 95% or more, for example, 98% or 99% identity with the full length native polypeptide sequence. Variants may also include variant fragments of a reference, e.g., native, sequence sharing a sequence identity of 70% or more with a fragment of the reference, e.g., native, sequence, e.g., an identity of 75% or 80% or more, such as 85%, 90%, or 95% or more, for example, 98% or 99% identity with the native sequence.

The term “stem cell” as used herein refers to a mammalian cell that has the ability both to self-renew, and to generate differentiated progeny (see Morrison et al. (1997) Cell 88:287-298). Endogenous stem cells may be characterized by the presence of markers associated with specific epitopes. Hematopoietic stem cells (HSC) are multipotent cells that reside in the bone marrow (BM) and are responsible for the life-long production of mature blood cells. HSPCs include HSCs as well as hematopoietic progenitor cells that reside in bone marrow and are capable of differentiating into mature blood cells. In some embodiments, HSC and/or HSPC engraftment cells may be fresh, frozen, or subject to prior culture. HSC and/or HSPC may be obtained from fetal liver, bone marrow, cord blood, or peripheral blood, by a donor (allogeneic), the patient themselves (autologous), or any other conventional source.

The terms “administering” or “introducing” or “providing”, as used herein, refer to delivery of a composition to a cell, to cells, tissues and/or organs of a subject, or to a subject. Such administering or introducing may take place in vivo, in vitro or ex vivo.

The terms “treatment”, “treating” and the like are used herein to generally mean obtaining a desired pharmacologic and/or physiologic effect. The effect may be prophylactic in terms of completely or partially preventing a disease or symptom thereof, e.g., reducing the likelihood that the disease or symptom thereof occurs in the subject, and/or may be therapeutic in terms of a partial or complete cure for a disease and/or adverse effect attributable to the disease. “Treatment” as used herein covers any treatment of a disease in a mammal, and includes: (a) inhibiting the disease, i.e., arresting its development; or (b) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy may be administered before or during a symptomatic stage of the disease.

The terms “individual,” “host,” “subject,” and “patient” are used interchangeably herein, and refer to a mammal, including, but not limited to, human and non-human primates, including simians and humans; mammalian sport animals (e.g., horses), mammalian farm animals (e.g., sheep, goats, etc.), mammalian pets (dogs, cats, etc.), and rodents (e.g., mice, rats, etc.).

As used herein, the term “substantially” means by a significant or large amount or degree. For example, to “substantially” increase may mean to increase by at least two-fold, at least three-fold, at least four-fold, at least five-fold, or at least ten-fold, and to “substantially” decrease may mean to decrease by at least 30%, at least 40%, at least 50%, at least 60%, at least 70%, at least 80%, or at least 90%.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Generally, conventional methods of protein synthesis, recombinant cell culture and protein isolation, and recombinant DNA techniques within the skill of the art are employed in the present invention. Such techniques are explained fully in the literature, see, e.g., Maniatis, Fritsch & Sambrook, Molecular Cloning: A Laboratory Manual (1982); Sambrook, Russell and Sambrook, Molecular Cloning: A Laboratory Manual (2001); Harlow, Lane and Harlow, Using Antibodies: A Laboratory Manual: Portable Protocol No. I, Cold Spring Harbor Laboratory (1998); and Harlow and Lane, Antibodies: A Laboratory Manual, Cold Spring Harbor Laboratory; (1988).

### CD117 Variant Polypeptides

CD117, also known as CD117 or stem cell factor receptor (SCFR), has a molecular weight of 145 kDa as a mature protein and is a member of the type III receptor tyrosine kinase (RTK) family that includes platelet-derived growth factor (PDGF) receptors and the macrophage colony-stimulating factor 1 (CSF-1) (c-fms) receptor. CD117 is essential for the development of normal hematopoietic cells and plays an important role in the survival, proliferation, and differentiation of mast cells, melanocytes, and germ cells. It is expressed by hematopoietic cells in the embryonic liver throughout development, and by more committed progenitors, such as myeloid, erythroid, megakaryocytic, natural killer, and dendritic progenitor cells.

CD117 includes an approximately 519 amino acid extracellular domain comprised of five immunoglobulin-like domains, a transmembrane segment, a juxtamembrane domain, and a protein kinase domain that contains an insert of about 80 amino acid residues. Approximately 184 amino acids of the extracellular domain are surface exposed, which were identified based on x-ray crystallographic studies. The crystallographic structure of CD117 is provided in, e.g., Mol, et al., Accelerated Publications, Volume 278, ISSUE 34, P31461-31464, Aug. 22, 2003; Ogg et al., RCSB Protein Data Bank, 6XV9, Crystal structure of the kinase domain of human CD117 in complex with a type-II inhibitor, DOI: 10.2210/pdb6XV9/pdb; McAuley et al., RCSB Protein Data Bank Alkynyl Benzoxazines and Dihydroquinazolines as Cysteine Targeting Covalent Warheads and Their Application in Identification of Selective Irreversible Kinase Inhibitors, DOI: 10.1021/jacs.9b13391; Schimpl et al., RCSB Protein Data Bank 6GQM, Crystal structure of human CD117 kinase domain in complex with a small molecule inhibitor, AZD3229, DOI: 10.1021/acs.jmedchem.8b00938; and Lin et al., RCSB Protein Data Bank Identification of a Multitargeted Tyrosine Kinase Inhibitor for the Treatment of Gastrointestinal Stromal Tumors and Acute Myeloid Leukemia, DOI: 10.1021/acs.jmedchem.9b01229. Binding of CD117 to its ligand (stem cell factor; SCF) induces receptor dimerization, trans autophosphorylation of the kinase domain, recruitment of signaling proteins via phosphotyrosine binding or Src homology 2 (SH2) domains, and subsequent signal transduction.

In particular embodiments, an illustrative SCF has the following polypeptide sequence: NP_000890.1 kit ligand isoform b precursor [Homo sapiens]

(SEQ ID NO: 14)
MKKTQTWILTCIYLQLLLFNPLVKTEGICRNRVTNNVKDVTKLVANLPKDYMITLKYVP
GMDVLPSHCWISEMVVQLSDSLTDLLDKFSNISEGLSNYSIIDKLVNIVDDLVECVKENS
SKDLKKSFKSPEPRLFTPEEFFRIFNRSIDAFKDFWASETSDCWSSTLSPEKDSRVSVTKPF
MLPPVAASSLRNDSSSSNRKAKNPPGDSSLHWAAMALPALFSLIIGFAFGALYWKKRQP
SLTRAVENIQINEEDNEISMLQEKEREFQEV.

In one aspect, the disclosure provides a modified CD117 polypeptide comprising one or more amino acid modifications as compared to a wild type CD117 polypeptide. In particular embodiments, the one or more amino acid modifications comprise one or more amino acid substitutions, insertions, or deletions. In certain embodiments, the one or more amino acid modifications are located in the extracellular domain of the CD117 polypeptide. In certain embodiments, the one or more amino acid modifications are located in one or more surface exposed amino acids of the CD117 polypeptide's extracellular domain. In particular embodiments, the modified CD117 polypeptides comprise one or more deletions, e.g., an N-terminal or C-terminal deletion, optionally wherein the deletion does not substantially impair biological activity, e.g., signaling, of the modified CD117 polypeptide, e.g., in response to SCF binding to the modified CD117 polypeptide when expressed in cells, e.g., HSCs and/or HSPCs. In certain embodiments, the modified CD117 polypeptides retain or have at least 90%, at least 95%, at least 98%, or at least 99% sequence homology to the wild type CD117 polypeptide.

In particular embodiments, the one or more amino acid modifications inhibit or reduce binding of an anti-CD117 antibody to the modified CD117 polypeptide expressed in cells as compared to binding of the antibody to the wild type CD117 polypeptide. In particular embodiments, the modified CD117 has less than 10%, less than 20%, less than 30%, less than 40%, less than 50%, less than 60%, or less than 70% binding to an anti-CD117 antibody as compared to the corresponding wild type CD117.

Thus, in certain embodiments, the one or more amino acid modifications disrupt or are present within an epitope of wild type CD117 that is bound by an anti-CD117 antibody. In particular embodiments, the anti-CD117 antibody comprises the six CDRs present in any one of JSP191, AB85, CDX-0159, or FSI-174. In particular embodiments, the anti-CD117 antibody in any one of JSP191, AB85, CDX-0159, or FSI-174.

In some embodiments, the anti-CD117 antibody is JSP191 or comprises one or more (e.g., 2, 3, 4, 5, or 6) of the six CDRs present in JSP191, and the one or more amino acid modifications are present within one or more epitope on wild type CD117 bound by JSP191. In certain embodiments, the modified CD117 comprises an amino acid modification, e.g., a substitution, of one or more of E73, D121, R122, S123, Y125, and K203, or is within an epitope comprising any of these amino acids. In certain embodiments, the substitution is an alanine substitution, a conservative substitution, or a non-conservative substitution.

In some embodiments, the anti-CD117 antibody is AB85 or comprises one or more (e.g., 2, 3, 4, 5, or 6) of the six CDRs present in AB85, and the one or more amino acid modifications are present within one or more epitope on CD117 bound by AB85. In certain embodiments, the modified CD117 comprises an amino acid modification, e.g., a substitution, of one or more of Y259, S261, W262, Y269, and R271, or is within an epitope comprising any of the amino acids. In certain embodiments, the substitution is an alanine substitution, a conservative substitution, or a non-conservative substitution.

In some embodiments, the anti-CD117 antibody is CDX-0159 or comprises one or more (e.g., 2, 3, 4, 5, or 6) of the six CDRs present in CDX-0159, and the one or more amino acid modifications are present within one or more epitope on CD117 bound by CDX-0159. In certain embodiments, the substitution is an alanine substitution, a conservative substitution, or a non-conservative substitution.

In some embodiments, the anti-CD117 antibody is FSI-174 or comprises one or more (e.g., 2, 3, 4, 5, or 6) of the six CDRs present in FSI-174, and the one or more amino acid modifications are present within one or more epitope on CD117 bound by FSI-174. In certain embodiments, the substitution is an alanine substitution, a conservative substitution, or a non-conservative substitution.

In particular embodiments, the one or more amino acid modifications comprise one or more amino acid substitutions or deletions of an amino acid residue selected from the following in human CD117: E73, D121, R122, S123, Y125, K203, Y259, S261, W262, Y269, or R271. In certain embodiments, the one or more amino acid modifications comprise one or more amino acid substitutions, e.g., of any of these residues.

In certain embodiments, an amino acid residue is substituted by any other amino acid, by alanine. In certain embodiments, the amino acid substitution is a conservative amino acid substitution. The term “conservative substitution” as used herein denotes that one or more amino acids are replaced by another, biologically similar residue.

In the scheme below, conservative substitutions of amino acids are grouped by the indicated physicochemical properties. I: neutral, hydrophilic, II: acids and amides, III: basic, IV: hydrophobic, V: aromatic, bulky amino acids.

| I | II | III | IV | V |
|---|---|---|---|---|
| A | N | H | M | F |
| S | D | R | L | Y |
| T | E | K | I | W |
| P | Q | | V | |
| G | | | C | |

In the scheme below, conservative substitutions of amino acids are grouped by the indicated physicochemical properties. VI: neutral or hydrophobic, VII: acidic, VIII: basic, IX: polar, X: aromatic.

| VI | VII | VIII | IX | X |
|---|---|---|---|---|
| A | E | H | M | F |
| L | D | R | S | Y |
| I | | K | T | W |
| P | | | C | |
| G | | | N | |
| V | | | Q | |

In certain embodiments, the wild type CD117 polypeptide upon which the variant is based is a human CD117 polypeptide, while in other embodiments, it is another mammalian CD117polypeptide. Sequences of human and mammalian CD117 polypeptides are known in the art. Due to alternative splicing of the CD117, aka c-KIT, gene, the human CD117 polypeptide is expressed as various isoforms, and any of these may be used according to the disclosure. These isoforms include two GNNK+ and GNNK− isoforms (also denoted c-Kit and c-KitA, respectively), which differ by the presence or absence of four amino acids, GNNK, and which are coexpressed in most tissues, although the GNNK− isoform usually predominates. In particular embodiments, the wild type CD117 polypeptide is the GNNK+ or GNNK− isoform and comprises or consists of one of the following amino acid sequences:

(SEQ ID NO: 1)
MRGARGAWDFLCVLLLLLRVQTGSSQPSVSPGEPSPPSIHPGKSDLIVRVGDEIRLLCTDP
GFVKWTFEILDETNENKQNEWITEKAEATNTGKYTCTNKHGLSNSIYVFVRDPAKLFLV
DRSLYGKEDNDTLVRCPLTDPEVTNYSLKGCQGKPLPKDLRFIPDPKAGIMIKSVKRAY
HRLCLHCSVDQEGKSVLSEKFILKVRPAFKAVPVVSVSKASYLLREGEEFTVTCTIKDVS
SSVYSTWKRENSQTKLQEKYNSWHHGDFNYERQATLTISSARVNDSGVFMCYANNTFG
SANVTTTLEVVDKGFINIFPMINTTVFVNDGENVDLIVEYEAFPKPEHQQWIYMNRTFTD
KWEDYPKSENESNIRYVSELHLTRLKGTEGGTYTFLVSNSDVNAAIAFNVYVNTKPEILT
YDRLVNGMLQCVAAGFPEPTIDWYFCPGTEQRCSASVLPVDVQTLNSSGPPFGKLVVQS
SIDSSAFKHNGTVECKAYNDVGKTSAYFNFAFKGNNKEQIHPHTLFTPLLIGFVIVAGM
MCIIVMILTYKYLQKPMYEVQWKVVEEINGNNYVYIDPTQLPYDHKWEFPRNRLSFGK
TLGAGAFGKVVEATAYGLIKSDAAMTVAVKMLKPSAHLTEREALMSELKVLSYLGNH
MNIVNLLGACTIGGPTLVITEYCCYGDLLNFLRRKRDSFICSKQEDHAEAALYKNLLHSK
ESSCSDSTNEYMDMKPGVSYVVPTKADKRRSVRIGSYIERDVTPAIMEDDELALDLEDL
LSFSYQVAKGMAFLASKNCIHRDLAARNILLTHGRITKICDFGLARDIKNDSNYVVKGN
ARLPVKWMAPESIFNCVYTFESDVWSYGIFLWELFSLGSSPYPGMPVDSKFYKMIKEGF
RMLSPEHAPAEMYDIMKTCWDADPLKRPTFKQIVQLIEKQISESTNHIYSNLANCSPNRQ
KPVVDHSVRINSVGSTASSSQPLLVHDDV;
or
(SEQ ID NO: 2)
MRGARGAWDFLCVLLLLLRVQTGSSQPSVSPGEPSPPSIHPGKSDLIVRVGDEIRLLCTDP
GFVKWTFEILDETNENKQNEWITEKAEATNTGKYTCTNKHGLSNSIYVFVRDPAKLFLV
DRSLYGKEDNDTLVRCPLTDPEVTNYSLKGCQGKPLPKDLRFIPDPKAGIMIKSVKRAY
HRLCLHCSVDQEGKSVLSEKFILKVRPAFKAVPVVSVSKASYLLREGEEFTVTCTIKDVS
SSVYSTWKRENSQTKLQEKYNSWHHGDFNYERQATLTISSARVNDSGVFMCYANNTFG
SANVTTTLEVVDKGFINIFPMINTTVFVNDGENVDLIVEYEAFPKPEHQQWIYMNRTFTD
KWEDYPKSENESNIRYVSELHLTRLKGTEGGTYTFLVSNSDVNAAIAFNVYVNTKPEILT
YDRLVNGMLQCVAAGFPEPTIDWYFCPGTEQRCSASVLPVDVQTLNSSGPPFGKLVVQS
SIDSSAFKHNGTVECKAYNDVGKTSAYFNFAFKEQIHPHTLFTPLLIGFVIVAGMMCIIV
MILTYKYLQKPMYEVQWKVVEEINGNNYVYIDPTQLPYDHKWEFPRNRLSFGKTLGAG
AFGKVVEATAYGLIKSDAAMTVAVKMLKPSAHLTEREALMSELKVLSYLGNHMNIVN
LLGACTIGGPTLVITEYCCYGDLLNFLRRKRDSFICSKQEDHAEAALYKNLLHSKESSCS
DSTNEYMDMKPGVSYVVPTKADKRRSVRIGSYIERDVTPAIMEDDELALDLEDLLSFSY
QVAKGMAFLASKNCIHRDLAARNILLTHGRITKICDFGLARDIKNDSNYVVKGNARLPV
KWMAPESIFNCVYTFESDVWSYGIFLWELFSLGSSPYPGMPVDSKFYKMIKEGFRMLSP
EHAPAEMYDIMKTCWDADPLKRPTFKQIVQLIEKQISESTNHIYSNLANCSPNRQKPVVD
HSVRINSVGSTASSSQPLLVHDDV.

In certain embodiments, the modified CD117 polypeptide substantially retains kinase activity as compared to the wild type CD117 polypeptide, e.g., when expressed in cells and bound by SCF. In particular embodiments, the modified CD117 polypeptide has at least 50%, at least 60%, at least 70%, at least 80%, at least 90%, or 100% of the kinase activity of the wild type CD117 polypeptide. Kinase activity may be determined using assays known in the art, including the ADP-Glo™ Kinase Assay, which is a luminescent kinase assay that measures ADP formed from a kinase reaction; ADP is converted into ATP, which is converted into light by Ultra-Glo™ Luciferase (available from Promega Corporation, Madison, WI).

In particular embodiments, the one or more amino acid modifications do not substantially inhibit or reduce binding of stem cell factor (SCF) to the modified CD117 polypeptide when expressed in cells, as compared to the binding of SCF to the wild type CD117 polypeptide. In particular embodiments, the modified CD117 retains at least 40%, at least 50%, at least 60%, at least 70%, at least 80%, at least 90%, or at least 95% of binding to SCF as compared to the corresponding wild type CD117.

In particular embodiments, the one or more amino acid modifications do not result in cells expressing only the modified CD117 having substantially inhibited or reduce CD117 signaling or proliferation or viability, optionally in response to SCF binding, as compared to the signaling in cells only expressing the wild type CD117 polypeptide. In particular embodiments, the modified CD117 retains at least 40%, at least 50%, at least 60%, at least 70%, at least 80%, at least 90%, or at least 95% CD117 signaling and/or proliferation and/or viability, optionally in response to SCF binding, as compared to the corresponding wild type CD117.

CD117 signaling or proliferation or viability may be determined using methods standard in the art. For example, in certain embodiments, CD117 signaling or proliferation (e.g., in response to SCF), of cells comprising a modified CD117 polypeptide is determined using a cell line (e.g., Ba/F3 cells) engineered to express the modified CD117 polypeptide. Cells are cultured in the presence of IL-3, with or without stem cell factor (SCF), and in the presence or absence of an anti-CD117 antibody, e.g., JSP191. Control parental Ba/F3 cells do not proliferate in the absence of IL-3. Further, parental Ba/F3 cells do not express CD117 and are not responsive to SCF signaling. Proliferation in response to SCF binding may this be determined for cells overexpressing the modified CD117, e.g., in the presence and absence of SCF and/or the anti-CD117 antibody.

### CD117 Polynucleotides

The disclosure also provides nucleic acid or polynucleotides, e.g., messenger RNA (mRNA) encoding a wild type or modified CD117 polypeptide.

In particular embodiments, the nucleic acid comprises RNA, DNA, or a combination thereof, and in particular embodiments, the nucleic acid comprises single-stranded and/or double-stranded regions, or a mixture thereof. In certain embodiments, the nucleic acid is a double-stranded DNA, and in certain embodiments, the nucleic acid is a single stranded RNA, e.g., a messenger RNA (mRNA).

In some embodiments, an RNA, e.g., an mRNA of the disclosure may be produced by in vitro transcription (IVT) of one or more DNA templates having polynucleotide sequence(s) encoding the desired RNA. The DNA template may comprise one or more promoters that enable transcription. In some embodiments, for example, a template may comprise a T7 promoter configured for transcription of the CD117 mRNA. Alternatively, the RNA may be prepared synthetically. The disclosure includes DNA templates comprising a sequence disclosed herein, e.g., any of SEQ ID NOs: 5-7, and variants thereof. In certain embodiments, the DNA template sequences are present in a DNA molecule or a DNA vector or plasmid.

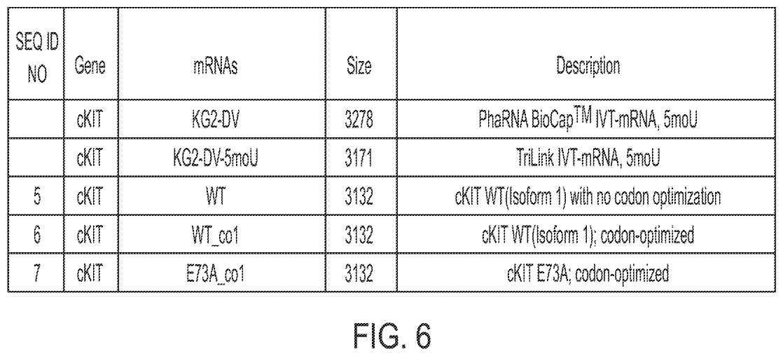

In some embodiments, a nucleic acid comprises any of the sequences of FIG. 6 (SEQ ID NOs: 5-7), or a sequence with at least 50%, at least 55%, at least 60%, at least 70%, at least 75%, at least 80%, at least 85%, at least 90%, at least 95%, at least 96%, at least 97%, at least 98%, or at least 99%, sequence identity thereto. In certain embodiments, a nucleic acid, e.g., an mRNA, comprises the coding region of any of the sequences of FIG. 6 (SEQ ID NOS: 5-7), or a sequence with at least 50%, at least 55%, at least 60%, at least 70%, at least 75%, at least 80%, at least 85%, at least 90%, at least 95%, at least 96%, at least 97%, at least 98%, or at least 99%, sequence identity thereto. In certain embodiments, a nucleic acid, e.g., an mRNA, is expressed from a template or vector comprising any of the sequences of FIG. 6 (SEQ ID NOS: 5-7), or a sequence with at least 50%, at least 55%, at least 60%, at least 70%, at least 75%, at least 80%, at least 85%, at least 90%, at least 95%, at least 96%, at least 97%, at least 98%, or at least 99%, sequence identity thereto. In certain embodiments, a nucleic acid, e.g., an mRNA, comprises any of the sequences of FIG. 6 (SEQ ID NOS: 5-7), or a sequence with at least 50%, at least 55%, at least 60%, at least 70%, at least 75%, at least 80%, at least 85%, at least 90%, at least 95%, at least 96%, at least 97%, at least 98%, or at least 99%, sequence identity thereto, absent the promoter and/or including a poly A sequence, e.g., an A70 polyA tail.

In particular embodiments, the polynucleotides described herein, e.g., an mRNA, are codon-optimized, e.g., to enhance expression of the encoded polypeptide in a host cell. It is understood that RNA disclosed herein may comprise any of the DNA sequences disclosed herein, or portions or variants thereof, wherein the Ts of the DNA are substituted with Us (or modifications thereof).

In particular embodiments, the mRNA comprises an RNA sequence corresponding to the open reading frame DNA sequence of SEQ ID NO: 5:

ATGAGAGGCGCTCGCGGCGCCTGGGATTTTCTCTGCGTTCTGCTCCTACTGCTTCGCG
TCCAGACAGGCTCTTCTCAACCATCTGTGAGTCCAGGGGAACCGTCTCCACCATCCA
TCCATCCAGGAAAATCAGACTTAATAGTCCGCGTGGGCGACGAGATTAGGCTGTTAT
GCACTGATCCGGGCTTTGTCAAATGGACTTTTGAGATCCTGGATGAAACGAATGAGA
ATAAGCAGAATGAATGGATCACGGAAAAGGCAGAAGCCACCAACACCGGCAAATA
CACGTGCACCAACAAACACGGCTTAAGCAATTCCATTTATGTGTTTGTTAGAGATCC
TGCCAAGCTTTTCCTTGTTGACCGCTCCTTGTATGGGAAAGAAGACAACGACACGCT
GGTCCGCTGTCCTCTCACAGACCCAGAAGTGACCAATTATTCCCTCAAGGGGTGCCA
GGGGAAGCCTCTTCCCAAGGACTTGAGGTTTATTCCTGACCCCAAGGCGGGCATCAT
GATCAAAAGTGTGAAACGCGCCTACCATCGGCTCTGTCTGCATTGTTCTGTGGACCA
GGAGGGCAAGTCAGTGCTGTCGGAAAAATTCATCCTGAAAGTGAGGCCAGCCTTCA
AAGCTGTGCCTGTTGTGTCTGTGTCCAAAGCAAGCTATCTTCTTAGGGAAGGGGAAG
AATTCACAGTGACGTGCACAATAAAAGATGTGTCTAGTTCTGTGTACTCAACGTGGA
AAAGAGAAAACAGTCAGACTAAACTACAGGAGAAATATAATAGCTGGCATCACGGT
GACTTCAATTATGAACGTCAGGCAACGTTGACTATCAGTTCAGCGAGAGTTAATGAT
TCTGGAGTGTTCATGTGTTATGCCAATAATACTTTTGGATCAGCAAATGTCACAACA
ACCTTGGAAGTAGTAGATAAAGGATTCATTAATATCTTCCCCATGATAAACACTACA
GTATTTGTAAACGATGGAGAAAATGTAGATTTGATTGTTGAATATGAAGCATTCCCC
AAACCTGAACACCAGCAGTGGATCTATATGAACAGAACCTTCACTGATAAATGGGA
AGATTATCCCAAGTCTGAGAATGAAAGTAATATCAGATACGTAAGTGAACTTCATCT
AACGAGATTAAAAGGCACCGAAGGAGGCACTTACACATTCCTAGTGTCCAATTCTG
ACGTCAATGCTGCCATAGCATTTAATGTTTATGTGAATACAAAACCAGAAATCCTGA
CTTACGACAGGCTCGTGAATGGCATGCTCCAATGTGTGGCAGCAGGATTCCCAGAGC
CCACAATAGATTGGTATTTTTGTCCAGGAACTGAGCAGAGATGCTCTGCTTCTGTAC
TGCCAGTGGATGTGCAGACACTAAACTCATCTGGGCCACCGTTTGGAAAGCTAGTGG
TTCAGAGTTCTATAGATTCTAGTGCATTCAAGCACAATGGCACGGTTGAATGTAAGG
CTTACAACGATGTGGGCAAGACTTCTGCCTATTTTAACTTTGCATTTAAAGGTAACA
ACAAAGAGCAAATCCATCCCCACACCCTGTTCACTCCTTTGCTGATTGGTTTCGTAAT
CGTAGCTGGCATGATGTGCATTATTGTGATGATTCTGACCTACAAATATTTACAGAA
ACCCATGTATGAAGTACAGTGGAAGGTTGTTGAGGAGATAAATGGAAACAATTATG
TTTACATAGACCCAACACAACTTCCTTATGATCACAAATGGGAGTTTCCCAGAAACA
GGCTGAGTTTTGGGAAAACCCTGGGTGCTGGAGCTTTCGGGAAGGTTGTTGAGGCA
ACTGCTTATGGCTTAATTAAGTCAGATGCGGCCATGACTGTCGCTGTAAAGATGCTC
AAGCCGAGTGCCCATTTGACAGAACGGGAAGCCCTCATGTCTGAACTCAAAGTCCT
GAGTTACCTTGGTAATCACATGAATATTGTGAATCTACTTGGAGCCTGCACCATTGG
AGGGCCCACCCTGGTCATTACAGAATATTGTTGCTATGGTGATCTTTTGAATTTTTTG
AGAAGAAAACGTGATTCATTTATTTGTTCAAAGCAGGAAGATCATGCAGAAGCTGC
ACTTTATAAGAATCTTCTGCATTCAAAGGAGTCTTCCTGCAGCGATAGTACTAATGA
GTACATGGACATGAAACCTGGAGTTTCTTATGTTGTCCCAACCAAGGCCGACAAAAG
GAGATCTGTGAGAATAGGCTCATACATAGAAAGAGATGTGACTCCCGCCATCATGG
AGGATGACGAGTTGGCCCTAGACTTAGAAGACTTGCTGAGCTTTTCTTACCAGGTGG
CAAAGGGCATGGCTTTCCTCGCCTCCAAGAATTGTATTCACAGAGACTTGGCAGCCA
GAAATATCCTCCTTACTCATGGTCGGATCACAAAGATTTGTGATTTTGGTCTAGCCA
GAGACATCAAGAATGATTCTAATTATGTGGTTAAAGGAAACGCTCGACTACCTGTGA
AGTGGATGGCACCTGAAAGCATTTTCAACTGTGTATACACGTTTGAAAGTGACGTCT
GGTCCTATGGGATTTTTCTTTGGGAGCTGTTCTCTTTAGGAAGCAGCCCCTATCCTGG
AATGCCGGTCGATTCTAAGTTCTACAAGATGATCAAGGAAGGCTTCCGGATGCTCAG
CCCTGAACACGCACCTGCTGAAATGTATGACATAATGAAGACTTGCTGGGATGCAG
ATCCCCTAAAAAGACCAACATTCAAGCAAATTGTTCAGCTAATTGAGAAGCAGATTT
CAGAGAGCACCAATCATATTTACTCCAACTTAGCAAACTGCAGCCCCAACCGACAG
AAGCCCGTGGTAGACCATTCTGTGCGGATCAATTCTGTCGGCAGCACCGCTTCCTCC
TCCCAGCCTCTGCTTGTGCACGACGATGTCT.

In particular embodiments, the mRNA comprises an RNA sequence corresponding to the open reading frame DNA sequence of SEQ ID NO: 6:

ATGAGAGGCGCCCGGGGCGCATGGGACTTCTTATGTGTATTATTGCTCCTCCTGAGG
GTTCAGACCGGGAGTAGCCAACCTTCCGTGTCCCCTGGTGAACCATCGCCACCATCG
ATCCACCCTGGCAAATCCGACCTGATCGTGAGGGTCGGCGATGAAATCCGGCTCCTA
TGCACGGACCCTGGTTTTGTGAAATGGACCTTTGAGATCCTCGATGAAACCAATGAG
AATAAACAGAACGAGTGGATTACAGAAAAGGCTGAGGCCACAAATACAGGTAAAT
ACACATGCACCAATAAGCACGGCCTCTCGAACTCTATCTACGTTTTTGTGCGAGATC
CTGCAAAACTTTTCCTCGTCGATCGCTCCTTATACGGAAAGGAGGACAATGATACAC
TGGTCAGATGCCCTTTAACAGACCCCGAGGTTACAAACTATTCACTCAAGGGTTGTC
AGGGTAAACCTCTGCCAAAAGACCTGCGCTTTATCCCTGATCCTAAAGCCGGCATCA
TGATAAAATCTGTAAAGCGGGCCTACCACCGTCTCTGCTTGCACTGTAGCGTTGATC
AAGAAGGCAAGTCAGTTTTAAGCGAGAAATTCATCCTGAAGGTGCGCCCAGCCTTC
AAGGCTGTGCCTGTAGTGTCAGTGTCTAAGGCCTCATACCTGCTCCGGGAGGGGGAG
GAATTTACTGTGACCTGCACGATAAAAGACGTTTCCTCTTCTGTGTACTCTACTTGGA
AGAGGGAAAATAGCCAGACCAAGCTGCAGGAGAAGTACAACAGCTGGCATCATGG
TGACTTCAACTACGAGAGACAGGCCACTCTGACTATTTCATCTGCACGCGTGAATGA
CTCCGGTGTGTTTATGTGCTACGCAAATAACACCTTCGGCAGTGCCAATGTGACAAC
TACCCTGGAAGTCGTTGACAAGGGCTTCATTAACATCTTTCCAATGATCAATACAAC
CGTCTTTGTTAACGATGGCGAAAACGTGGACCTGATCGTTGAATATGAAGCATTCCC
CAAACCAGAGCACCAGCAGTGGATCTACATGAATCGCACTTTCACCGACAAGTGGG
AAGACTACCCCAAATCCGAGAACGAGTCTAACATCCGCTATGTGTCGGAACTCCAC
CTGACTAGACTTAAGGGTACGGAAGGAGGCACCTACACCTTTTTGGTGAGCAATAG
CGACGTGAACGCGGCAATTGCTTTTAACGTATACGTGAATACGAAACCCGAAATATT
GACATATGACCGTCTGGTGAACGGAATGCTTCAGTGTGTGGCCGCAGGCTTTCCTGA
ACCAACCATCGACTGGTACTTTTGCCCTGGTACCGAGCAGCGGTGCTCCGCGAGCGT
GCTGCCTGTGGACGTCCAGACGCTAAATTCTAGTGGGCCACCTTTTGGAAAACTGGT
GGTTCAGTCGTCAATTGATTCTTCTGCATTTAAGCATAATGGGACAGTGGAGTGTAA
AGCTTACAACGATGTGGGGAAGACAAGCGCCTATTTCAACTTTGCCTTTAAGGGGAA
CAATAAAGAGCAGATTCATCCACACACCTTGTTCACTCCTTTATTGATCGGGTTTGTG
ATCGTGGCGGGAATGATGTGTATTATCGTTATGATTTTGACTTATAAGTACCTGCAG
AAGCCTATGTATGAAGTGCAGTGGAAAGTGGTGGAAGAGATCAACGGGAACAATTA
CGTTTATATCGACCCCACCCAGTTGCCATATGACCACAAATGGGAATTTCCCAGGAA
TCGCTTGAGCTTCGGGAAGACACTCGGTGCCGGAGCCTTCGGAAAGGTAGTAGAAG
CAACGGCTTACGGGCTAATCAAGTCAGATGCCGCTATGACTGTTGCGGTGAAAATGT
TGAAGCCATCGGCTCATCTGACAGAGCGGGAGGCTCTGATGAGCGAACTCAAGGTT
CTCAGTTACCTCGGCAATCACATGAACATTGTAAATCTCCTTGGGGCCTGTACGATC
GGCGGTCCCACCCTCGTCATAACAGAATACTGCTGCTATGGCGATCTGCTGAACTTC
CTCCGGCGCAAGAGGGATTCCTTTATATGTAGCAAACAAGAAGACCACGCGGAGGC
CGCTCTATACAAAAACCTGTTACACAGTAAAGAGTCTTCATGCAGCGACAGTACGA
ATGAATACATGGACATGAAACCTGGGGTAAGTTATGTTGTGCCTACTAAAGCCGAC
AAGCGGCGCAGCGTCAGGATCGGATCCTATATTGAGAGGGACGTGACACCTGCTAT
TATGGAAGATGACGAGTTAGCATTGGACCTCGAGGACCTTCTATCCTTTTCATATCA
GGTGGCCAAGGGCATGGCCTTCCTGGCGTCTAAAAACTGTATTCACCGCGATTTGGC
CGCGAGAAACATCCTGCTCACACATGGAAGAATCACCAAAATTTGCGACTTTGGCCT
GGCCAGAGATATCAAGAACGACTCAAACTATGTGGTGAAGGGAAATGCACGTCTGC
CCGTGAAGTGGATGGCACCAGAGTCAATCTTTAATTGTGTGTATACGTTCGAAAGTG
ATGTCTGGTCATACGGAATCTTCCTGTGGGAGTTGTTCTCCCTGGGGTCTTCCCCCTA
CCCAGGGATGCCTGTGGACTCTAAATTCTACAAGATGATCAAGGAGGGCTTCCGCAT
GTTATCACCAGAGCACGCACCCGCTGAGATGTACGATATTATGAAAACTTGCTGGGA
CGCTGATCCCCTAAAGCGGCCAACTTTCAAACAGATTGTTCAGCTTATTGAAAAGCA
GATCAGTGAATCTACAAACCATATCTATAGTAATCTGGCCAATTGCTCACCTAACCG
ACAGAAGCCCGTGGTTGATCACTCCGTTAGGATCAACTCTGTGGGCAGCACTGCAAG
CTCCAGTCAGCCCCTGCTTGTCCACGATGATGTC.

In particular embodiments, the mRNA comprises an RNA sequence corresponding to the open reading frame DNA sequence of SEQ ID NO: 7:

ATGAGAGGCGCCCGGGGCGCATGGGACTTCTTATGTGTATTATTGCTCCTCCTGAGG
GTTCAGACCGGGAGTAGCCAACCTTCCGTGTCCCCTGGTGAACCATCGCCACCATCG
ATCCACCCTGGCAAATCCGACCTGATCGTGAGGGTCGGCGATGAAATCCGGCTCCTA
TGCACGGACCCTGGTTTTGTGAAATGGACCTTTGAGATCCTCGATGccACCAATGAGA
ATAAACAGAACGAGTGGATTACAGAAAAGGCTGAGGCCACAAATACAGGTAAATA
CACATGCACCAATAAGCACGGCCTCTCGAACTCTATCTACGTTTTTGTGCGAGATCC
TGCAAAACTTTTCCTCGTCGATCGCTCCTTATACGGAAAGGAGGACAATGATACACT
GGTCAGATGCCCTTTAACAGACCCCGAGGTTACAAACTATTCACTCAAGGGTTGTCA
GGGTAAACCTCTGCCAAAAGACCTGCGCTTTATCCCTGATCCTAAAGCCGGCATCAT
GATAAAATCTGTAAAGCGGGCCTACCACCGTCTCTGCTTGCACTGTAGCGTTGATCA
AGAAGGCAAGTCAGTTTTAAGCGAGAAATTCATCCTGAAGGTGCGCCCAGCCTTCA
AGGCTGTGCCTGTAGTGTCAGTGTCTAAGGCCTCATACCTGCTCCGGGAGGGGGAGG
AATTTACTGTGACCTGCACGATAAAAGACGTTTCCTCTTCTGTGTACTCTACTTGGAA
GAGGGAAAATAGCCAGACCAAGCTGCAGGAGAAGTACAACAGCTGGCATCATGGT
GACTTCAACTACGAGAGACAGGCCACTCTGACTATTTCATCTGCACGCGTGAATGAC
TCCGGTGTGTTTATGTGCTACGCAAATAACACCTTCGGCAGTGCCAATGTGACAACT
ACCCTGGAAGTCGTTGACAAGGGCTTCATTAACATCTTTCCAATGATCAATACAACC
GTCTTTGTTAACGATGGCGAAAACGTGGACCTGATCGTTGAATATGAAGCATTCCCC
AAACCAGAGCACCAGCAGTGGATCTACATGAATCGCACTTTCACCGACAAGTGGGA
AGACTACCCCAAATCCGAGAACGAGTCTAACATCCGCTATGTGTCGGAACTCCACCT
GACTAGACTTAAGGGTACGGAAGGAGGCACCTACACCTTTTTGGTGAGCAATAGCG
ACGTGAACGCGGCAATTGCTTTTAACGTATACGTGAATACGAAACCCGAAATATTGA
CATATGACCGTCTGGTGAACGGAATGCTTCAGTGTGTGGCCGCAGGCTTTCCTGAAC
CAACCATCGACTGGTACTTTTGCCCTGGTACCGAGCAGCGGTGCTCCGCGAGCGTGC
TGCCTGTGGACGTCCAGACGCTAAATTCTAGTGGGCCACCTTTTGGAAAACTGGTGG
TTCAGTCGTCAATTGATTCTTCTGCATTTAAGCATAATGGGACAGTGGAGTGTAAAG
CTTACAACGATGTGGGGAAGACAAGCGCCTATTTCAACTTTGCCTTTAAGGGGAACA
ATAAAGAGCAGATTCATCCACACACCTTGTTCACTCCTTTATTGATCGGGTTTGTGAT
CGTGGCGGGAATGATGTGTATTATCGTTATGATTTTGACTTATAAGTACCTGCAGAA
GCCTATGTATGAAGTGCAGTGGAAAGTGGTGGAAGAGATCAACGGGAACAATTACG
TTTATATCGACCCCACCCAGTTGCCATATGACCACAAATGGGAATTTCCCAGGAATC
GCTTGAGCTTCGGGAAGACACTCGGTGCCGGAGCCTTCGGAAAGGTAGTAGAAGCA
ACGGCTTACGGGCTAATCAAGTCAGATGCCGCTATGACTGTTGCGGTGAAAATGTTG
AAGCCATCGGCTCATCTGACAGAGCGGGAGGCTCTGATGAGCGAACTCAAGGTTCT
CAGTTACCTCGGCAATCACATGAACATTGTAAATCTCCTTGGGGCCTGTACGATCGG
CGGTCCCACCCTCGTCATAACAGAATACTGCTGCTATGGCGATCTGCTGAACTTCCT
CCGGCGCAAGAGGGATTCCTTTATATGTAGCAAACAAGAAGACCACGCGGAGGCCG
CTCTATACAAAAACCTGTTACACAGTAAAGAGTCTTCATGCAGCGACAGTACGAATG
AATACATGGACATGAAACCTGGGGTAAGTTATGTTGTGCCTACTAAAGCCGACAAG
CGGCGCAGCGTCAGGATCGGATCCTATATTGAGAGGGACGTGACACCTGCTATTATG
GAAGATGACGAGTTAGCATTGGACCTCGAGGACCTTCTATCCTTTTCATATCAGGTG
GCCAAGGGCATGGCCTTCCTGGCGTCTAAAAACTGTATTCACCGCGATTTGGCCGCG
AGAAACATCCTGCTCACACATGGAAGAATCACCAAAATTTGCGACTTTGGCCTGGCC
AGAGATATCAAGAACGACTCAAACTATGTGGTGAAGGGAAATGCACGTCTGCCCGT
GAAGTGGATGGCACCAGAGTCAATCTTTAATTGTGTGTATACGTTCGAAAGTGATGT
CTGGTCATACGGAATCTTCCTGTGGGAGTTGTTCTCCCTGGGGTCTTCCCCCTACCCA
GGGATGCCTGTGGACTCTAAATTCTACAAGATGATCAAGGAGGGCTTCCGCATGTTA
TCACCAGAGCACGCACCCGCTGAGATGTACGATATTATGAAAACTTGCTGGGACGCT
GATCCCCTAAAGCGGCCAACTTTCAAACAGATTGTTCAGCTTATTGAAAAGCAGATC
AGTGAATCTACAAACCATATCTATAGTAATCTGGCCAATTGCTCACCTAACCGACAG
AAGCCCGTGGTTGATCACTCCGTTAGGATCAACTCTGTGGGCAGCACTGCAAGCTCC
AGTCAGCCCCTGCTTGTCCACGATGATGTC.

In certain embodiments, the nucleic acid comprises a modified mRNA. In particular embodiments, a modified mRNA comprises one or more modified nucleotide or nucleoside. Modified mRNAs comprising one or more modified nucleoside have been described as having advantages over unmodified mRNAs, including increase stability, higher expression levels and reduced immunogenicity. Non-limiting examples of modifications to mRNAs that may be present in the nucleic acids encoding the modified CD117 polypeptides are described, e.g., in PCT Patent Application Publication Nos. WO2011/130624, WO2012/138453, WO2013052523, WO2013151666, WO2013/071047, WO2013/078199, WO2012045075, WO2014081507, WO2014093924, WO2014164253, US Patent Nos: U.S. Pat. No. 8,278,036 (describing modified mRNAs comprising pseudouridine), U.S. Pat. No. 8,691,966 (describing modified mRNAs comprising pseudouridine and/or N1-methylpseudouridine), U.S. Pat. No. 8,835,108 (describing modified mRNAs comprising 5-methylcytidine, U.S. Pat. No. 8,748,089 (describing modified mRNAs comprising pseudouridine or 1-methylpseudouridine). In particular embodiments, the modified mRNA comprises one or more nucleoside modification. In particular embodiments, the modified mRNA sequence comprises at least one modification as compared to an unmodified A, G, U or C ribonucleoside. For example, uridine can a similar nucleoside such as pseudouridine (Ψ) or N1-methyl-pseudouridine (m1Ψ), and cytosine can be replaced by 5-methylcytosine. In particular embodiments, the at least one modified nucleosides include N1-methyl-pseudouridine and/or 5-methylcytidine. In certain embodiments, all uridines in the modified mRNA are replaced with a similar nucleoside such as pseudouridine (Ψ) or N1-methyl-pseudouridine (m1Ψ), and/or all cytosines in the modified mRNA are substituted with a similar nucleoside such as 5-methylcytosine.

In particular embodiments, the modified mRNA comprises a 5′ terminal cap sequence followed by a sequence encoding the modified CD117 polypeptide, followed by a 3′ tailing sequence, such as a polyA or a polyA-G sequence.

In particular embodiments, the nucleic acid sequence (e.g., mRNA) encoding CD117 comprises 5′ and/or 3′ cellular or viral untranslated regions (UTRs) relative to the sequence encoding the CD117 polypeptide. In some embodiments, the UTR improves mRNA stability, localization and/or expression. In some embodiments, the UTR is tissue specific. In some embodiments, the 5′ UTR comprises a UTR sequence from alpha-globin. In some embodiments, the nucleic acid comprises a Kozak sequence. In some embodiments, the 3′UTR comprises a UTR from an alpha-globin and/or a beta-globin gene, i.e., a 5′ UTR from hemoglobin alpha 1 (HBA1) and/or a 3′ UTR from one or more of HBA1 or hemoglobin beta 1 (HBB1) gene. In some embodiments the modified mRNA comprises a 3′UTR and/or 5′UTR from HBA1 and/or HBB1. In some embodiments, the modified mRNA comprises a 3′ and/or 5′ UTR from AES and/or mtRNR1.

In some embodiments, the nucleic acid sequence encoding CD117 comprises a 5′ UTR with at least 90%, at least 95%, at least 96%, at least 97%, at least 98%, at least 99% identity, or 100% identity to a 5′UTR sequence of HBA1:

(SEQ ID NO: 15)
ACTCTTCTGGTCCCCACAGACTCAGAGAGAACCCACC.

In some embodiments, the nucleic acid sequence encoding CD117 comprises a Kozak sequence with at least 90%, at least 95%, at least 96%, at least 97%, at least 98%, at least 99% identity, or 100% identity to the following:
GCCGCCACC.

In some embodiments, the nucleic acid sequence encoding CD117 comprises a 3′UTR nucleic acid sequence with at least 90%, at least 95%, at least 96%, at least 97%, at least 98%, at least 99% identity, or 100% identity to a 3′UTR of HBB1:

(SEQ ID NO: 16)
GCTCGCTTTCTTGCTGTCCAATTTCTATTAAAGGTTCCTTTGTTCCCTA
AGTCCAACTACTAAACTGGGGGATATTATGAAGGGCCTTGAGCATCTGG
ATTCTGCCTAATAAAAAACATTTATTTTCATTGC.

In some embodiments, the nucleic acid sequence encoding CD117 comprises a 3′UTR nucleic acid sequence with at least 90%, at least 95%, at least 96%, at least 97%, at least 98%, at least 99% identity, or 100% to a 3′UTR of HBA1:

(SEQ ID NO: 17)
GCTGGAGCCTCGGTGGCCATGCTTCTTGCCCCTTGGGCCTCCCCCCAGC
CCCTCCTCCCCTTCCTGCACCCGTACCCCCGTGGTCTTTGAATAAAGTC
TGAGTGGGCGGCA.

In some embodiments, the nucleic acid sequence encoding CD117 comprises an extra stop codon downstream of TAA to avoid run-off translation of an mRNA. In some embodiments, the extra stop codon is TGA. In some embodiments, the nucleic acid sequence comprises a TAAATG double stop codon. In some embodiments the nucleic acid sequence encoding CD117 comprises a TCTAGA sequence to linearize a plasmid as a template for transcription.

In some embodiments, the nucleic acid sequence encoding CD117 encodes a poly-adenine or poly guanine tail. A poly A or polyA-G tail improves mRNA stability and manufacturability. In some embodiments, the polyA tail may be from 20 to 180 adenine bases in length. In some embodiments, the polyA tail may be from 35 to 140 bases in length. In some embodiments, the polyA tail is segmented with a linker to reduce recombination during plasmid production in prokaryotic cells. In some embodiments the polyA tail is 70 consecutive adenine bases in length (A70 poly A tail). In some embodiments, the linker is a series of bases other than adenine. In some embodiments, the linker is a series of bases including adenine. In some embodiments, the linker is about 3 to about 10 bases in length. In some embodiments, the linker is about 5 to about 20 bases in length. In some embodiments the linker comprises the sequence TATGCA.

In particular embodiments, the mRNA encoding wild type or mutant CD117 comprises a wild type 5′ terminal cap sequence. In certain embodiments, the mRNA encoding wild type or mutant CD117 comprises a modified 5′ terminal cap, not limited to but including, e.g., m7G(5′)ppp(5′)(2′OMeA)pG (CleanCap® Reagent AG for co-transcriptional capping of mRNA; TriLink Biotechnologies, USA) or m7(3′OMeG)(5′)ppp(5′)(2′OMeA)pG (CleanCap Reagent AG (3′ OMe) for co-transcriptional capping of mRNA; TriLink Biotechnologies, USA). In certain embodiments, the mRNA encoding CD47 comprises the modified 5′ terminal cap, 3′-O-Me-m7G(5′)ppp(5′)G (Anti Reverse Cap Analog (ARCA); APExBIO, USA).

In some embodiments, a modified mRNA comprises a viral polymerase promoter, e.g., a T7 CleanCapAG promoter. In some embodiments an mRNA encoding a modified wild type or mutant CD117 polypeptide comprise one or more of a T7 promoter, CleanCapAG, Kozak sequence, an HBA1 5′ UTR, a TAATGA double stop codon, an HBB1 3′ UTR, and a poly-adenosine tail, e.g., of 70 nucleotides. In particular embodiments, an mRNA construct comprises a CleanCapAG, Kozak sequence, HBA1 5′ UTR, open reading frame encoding CD117 or a mutant or modified CD117, e.g., E73A, a TAATGA double stop codon, and an A 70 poly A tail. In certain embodiments, it further comprises a HBB1 3′ UTR.

In certain embodiments, the nucleic acid, e.g., a modified mRNA, is associated with one or more lipids, e.g., to facilitate delivery across the cell membrane, shield its negative charge, and/or to protect against degradation by nucleases. In certain embodiments, the nucleic acid is associated with or present within a lipid nucleic acid particle, a lipid nanoparticle, or a liposome. In certain embodiments, the lipid nucleic acid particle, a lipid nanoparticle, or a liposome facilitates delivery or uptake of the nucleic acid by a cell. In certain embodiments, mRNA, optionally modified mRNA, is co-formulation into lipid nanoparticles (LNPs). In particular embodiments, mRNA-LNP formulations comprise: (1) an ionizable or cationic lipid or polymeric material bearing tertiary or quaternary amines to encapsulate the polyanionic mRNA; (2) a zwitterionic lipid (e.g., 1,2-dioleoyl-sn-glycero-3-phosphoethanolamine [DOPE]) that resembles the lipids in the cell membrane; (3) cholesterol to stabilize the lipid bilayer of the LNP; and (4) a polyethylene glycol (PEG)-lipid to lend the nanoparticle a hydrating layer, improve colloidal stability, and reduce protein absorption.

In certain embodiments, the nucleic acid encoding the modified CD117 polypeptide is present in a vector. In particular embodiments, the vector is capable of delivering the nucleic acid into mammalian HSCs or other stem cells, e.g., into the nucleus of the HSCs or stem cells. In certain embodiments, the vector is an episomal vector, e.g., a plasmid. In particular embodiments, the vector is an expression vector comprising a promoter sequence operatively linked to a nucleic acid sequence encoding the modified CD117 polypeptide. In particular embodiments, the expression vector comprises a promoter sequence that facilitates expression of the encoded modified CD117 polypeptide in HSCs or other stem cells. In particular embodiments, the expression vector comprises 5′ and/or 3′ cellular or viral UTRs or the derivatives thereof upstream and downstream, respectively, of the sequence encoding the modified CD117 polypeptide. In certain embodiments, the vector is used for in vitro transcription of an mRNA encoding the wild type or modified CD117 polypeptide.

In certain embodiments, the vector is a viral vector, optionally an AAV vector, a cytomegalovirus vector, an adenovirus vector, or a lentiviral vector. In certain embodiments, a viral vector infects an HSC when viral vector and the HSCs are incubated together for at least about 24 hours in a culture medium.

### Modified Hematopoietic Stem Cells and Pharmaceutical Compositions

In a related aspect, the disclosure provides modified cells, e.g., HSCs and/or HSPCs, comprising a nucleic acid encoding a modified CD117 polypeptide as described herein. In certain embodiments, the modified CD117 polypeptide comprises one or more amino acid substitutions, e.g., at one or more of the following amino acids present in wild type human CD117: E73, D121, R122, S123, Y125, K203, Y259, S261, W262, Y269, or R271. In particular embodiments, the modified CD117 polypeptide comprises one or modifications in an epitope of CD117 bound by an anti-CD117 antibody that comprises any of these amino acid residues. In particular embodiments, the modified CD117 polypeptide comprises a modification of an amino acid within 1, within 2, within 3, within 4, within 5, within 6, within 7, within 8, within 9, within 10, within 11, or within 12 amino acids of any of these amino acid residues. The modification may N-terminal or C-terminal to any of E73, D121, R122, S123, Y125, K203, Y259, S261, W262, Y269, or R271.

In certain embodiments, the nucleic acid, e.g., the mRNA or vector encoding the wild type or modified CD117 polypeptide is transiently present in the modified cell, and is not present within the genome of the cell. In other embodiments, the nucleic acid encoding the wild type or modified CD117 is incorporated into the genome of the cell. In certain embodiments, one or both endogenous CD117 alleles of the cell's genome are modified or edited to introduce the one or more modifications present in the modified CD117 polypeptide. In particular embodiments, the modified cell expresses and/or comprises the modified CD117 polypeptide, and in particular embodiments, the modified CD117 polypeptide is present on the cell surface, e.g., with the extracellular domain present outside the modified cell. In certain embodiments, the modified cell is transduced with or infected with an expression vector, optionally a viral vector. In particular embodiments, the modified cell expresses and/or comprises both the modified CD117 polypeptide and a wild type, endogenous CD117 polypeptide, and in particular embodiments, both the modified CD117 polypeptide and the wild type, endogenous CD117 polypeptide are present on the cell surface, e.g., with their extracellular domains present outside the modified cell. In certain embodiments, the modified cell expresses and/or comprises only the modified CD117 polypeptide and not a wild type, endogenous CD117 polypeptide, and in particular embodiments, the modified CD117 polypeptide is present on the cell surface, e.g., with its extracellular domain present outside the modified cell.

In certain embodiments, the modified cell comprising a modified CD117 polypeptide and/or encoding nucleic acid is a host cell, such as, e.g., an HEK293 cell that may be used to produce modified CD117 polypeptides. In preparing the subject compositions, any host cells may be employed, including but not limited to, for example, mammalian cells (e.g. 293 cells), insect cells (e.g., SF9 cells), microorganisms, and yeast.

In particular embodiments, the modified cell is a stem cell or pluripotent cell, and in certain embodiments, the stem cell is a hematopoietic stem cell (HSC) or an HSPC. In some embodiments, the stem cell is a mammalian cell that has the ability both to self-renew, and to generate differentiated progeny. In certain embodiments, the stem cell is a human cell. The stem cell may have one or more of the following properties: an ability to undergo asynchronous, or symmetric replication, that is where the two daughter cells after division can have different phenotypes; extensive self-renewal capacity; capacity for existence in a mitotically quiescent form; and clonal regeneration of all the tissue in which they exist, for example the ability of hematopoietic stem cells to reconstitute all hematopoietic lineages.

Hematopoietic stem cells (HSCs) are maintained throughout life (self-renewing). They produce hematopoietic progenitor cells that differentiate into every type of mature blood cell within a well-defined hierarchy. Hematopoietic stem cells can also be generated in vitro, for example from pluripotent embryonic stem cells, induced pluripotent cells, and the like. For example, see Sugimura et al. (2017) Nature 545:432-438, herein specifically incorporated by reference, which details a protocol for generation of hematopoietic progenitors.

The cells may be fresh, frozen, or have been subject to prior culture. They may be fetal, neonate, adult, etc. Hematopoietic stem cells and HSPCs may be obtained from fetal liver, bone marrow, blood, particularly G-CSF or GM-CSF mobilized peripheral blood, or any other conventional source. Cells for engraftment are optionally isolated from other cells, where the manner in which the stem cells are separated from other cells of the hematopoietic or other lineage is not critical to this invention. If desired, a substantially homogeneous population of stem or progenitor cells may be obtained by selective isolation of cells free of markers associated with differentiated cells, while displaying epitopic characteristics associated with the stem cells.

Modified HSCs may be produced using HSCs obtained from a mammalian donor. In particular embodiments, the donor is a subject in need of a hematopoietic stem cell transplant, e.g., a subject diagnosed with a disease or disorder that can be treated with HCT. In other embodiments, the modified HSCs may be produced using HSCs obtained from a healthy donor, e.g., wherein the modified HSCs are to be used to treat a different subject with HCT. Thus, the modified HSCs may be autologous or allogeneic to a subject in need for HCT.

Prior to harvesting stem cells from a donor, the bone marrow can be primed with granulocyte colony-stimulating factor (G-CSF; filgrastim [Neupogen]) to increase the stem cell count. Mobilization of stem cells from the bone marrow into peripheral blood by cytokines such as G-CSF or GM-CSF has led to the widespread adoption of peripheral blood progenitor cell collection by apheresis for hematopoietic stem cell transplantation. The dose of G-CSF used for mobilization may be about 10 ug/kg/day. In autologous donors who are heavily pretreated, however, doses of up to about 40 ug/kg/day can be given. Mozobil may be used in conjunction with G-CSF to mobilize hematopoietic stem cells to peripheral blood for collection.

Among hematopoietic stem cell (HSC) markers, CD34 is well known for its unique expression on HSCs. In certain embodiments, the modified cell is a CD34+cell. In particular embodiments, the modified cell is a subset of HSCs that has one of the following patterns or combinations of cell surface marker expression: CD34+/CD90+, CD34+/CD38−, or CD34+/CD38−/CD90+. The CD34+ and/or CD90+ cells may be selected by affinity methods, including without limitation magnetic bead selection, flow cytometry, and the like from the donor hematopoietic cell sample. The HSC composition may be at least about 50% pure, as defined by the percentage of cells that are CD34+ in the population, may be at least about 75% pure, at least about 85% pure, at least about 95% pure, or more.

In certain embodiments, the hematopoietic stem cells and/or HSPCs are obtained from bone marrow, peripheral blood, or umbilical cord blood and subsequently modified by introduction of the nucleic acid encoding the modified CD117 polypeptide into the cell. For example, the nucleic acid may be introduced by transfection or infection with a viral vector, or by contact with an mRNA, or the nucleic acid may be introduced by gene editing of an endogenous gene encoding CD117.

In certain embodiments, the disclosure provides a method of modifying cells, including stem cells such as HSCs and/or HSPCs, comprising introducing the nucleic acid encoding a modified CD117 polypeptide into the cell. In particular embodiments, the introduced nucleic acid is present within a viral vector. In certain embodiments, the nucleic acid is associated with or present in a lipid nanoparticle, liposome, or the like. In certain embodiments, the nucleic acid remains present in the modified cell only transiently, or the nucleic acid only transiently expresses the modified CD117 polypeptide in the cell. In certain embodiments, the method is used to prepare modified cells for HCT treatment of a mammalian subject. In particular embodiments, the nucleic acid or vector may be introduced into the cell by a variety of methods known in the art, such as transfection, transduction, infection, electroporation, or nanopore technology. In particular embodiments, mRNA, e.g., modified mRNA is introduced into the cells using lipid nucleic acid particles (LNPs) or nanoparticles. Thus, cells, e.g., HSCs and/or HSPCS may be modified by introducing a nucleic acid encoding a modified CD117 polypeptide into the HSCs and/or HSPCs according to a variety of methods available in the art. In particular embodiments, the nucleic acid, e.g., an mRNA or vector is introduced with electroporation.

In certain embodiments, the disclosure provides a method of modifying cells, including stem cells such as HSCs and/or HSPCs, comprising modifying one or more endogenous CD117 genes or alleles within the cells, e.g., by homologous recombination or gene editing according to a variety of methods available in the art. In certain embodiments, a CD117 gene in HSCs and/or HSPCs is edited by any of a variety of methods known and available in the art, including but not limited to: transcription activator-like effector nucleases (TALENs), megaTALs, clustered regularly interspaced short palindromic repeats (CRISPR)-CRISPR associated (Cas) systems, zinc finger nucleases, homing endonucleases, or meganucleases. In certain embodiments, the CD117 gene is edited by a base editing method. As used herein, a gene-editing system is a system comprising one or more proteins or polynucleotides capable of editing an endogenous target gene or locus in a sequence specific manner. In some embodiments, the gene-editing system is a protein-based gene regulating system comprising a protein comprising one or more zinc-finger binding domains and an enzymatic domain. In some embodiments, the protein-based gene regulating system comprises a protein comprising a Transcription activator-like effector nuclease (TALEN) domain and an enzymatic domain. Such embodiments are referred to herein as “TALENs.” In particular embodiments, the gene editing system comprises a nucleic acid sequence corresponding to a region of the CD117 gene and comprising a modification thereof.

Zinc finger-based systems comprise a fusion protein comprising two protein domains: a zinc finger DNA binding domain and an enzymatic domain. A “zinc finger DNA binding domain”, “zinc finger protein”, or “ZFP” is a protein, or a domain within a larger protein, that binds DNA in a sequence-specific manner through one or more zinc fingers, which are regions of amino acid sequence within the binding domain whose structure is stabilized through coordination of a zinc ion. The zinc finger domain, by binding to a target DNA sequence, directs the activity of the enzymatic domain to the vicinity of the sequence and, hence, induces modification of the endogenous target gene in the vicinity of the target sequence. A zinc finger domain can be engineered to bind to virtually any desired sequence. Accordingly, after identifying a target genetic locus containing a target DNA sequence at which cleavage or recombination is desired (e.g., a target locus or epitope identified herein), one or more zinc finger binding domains can be engineered to bind to one or more target DNA sequences in the target genetic locus. Expression of a fusion protein comprising a zinc finger binding domain and an enzymatic domain in a cell, effects modification in the target genetic locus.

In some embodiments, a zinc finger binding domain comprises one or more zinc fingers. Miller et al. (1985) EMBO J. 4:16010-1714; Rhodes (1993) Scientific American February:56-65; U.S. Pat. No. 6,453,242. Typically, a single zinc finger domain is about 30 amino acids in length. An individual zinc finger binds to a three-nucleotide (i.e., triplet) sequence (or a four-nucleotide sequence which can overlap, by one nucleotide, with the four-nucleotide binding site of an adjacent zinc finger). Therefore the length of a sequence to which a zinc finger binding domain is engineered to bind (e.g., a target sequence) will determine the number of zinc fingers in an engineered zinc finger binding domain. In some embodiments, the DNA-binding domains of individual ZFNs comprise between three and six individual zinc finger repeats and can each recognize between 9 and 18 base pairs.

Zinc finger binding domains can be engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416. An engineered zinc finger binding domain can have a novel binding specificity, compared to a naturally-occurring zinc finger protein. Engineering methods include, but are not limited to, rational design and various types of selection.

Selection of a target DNA sequence for binding by a zinc finger domain can be accomplished, for example, according to the methods disclosed in U.S. Pat. No. 6,453,242. It will be clear to those skilled in the art that simple visual inspection of a nucleotide sequence can also be used for selection of a target DNA sequence. Accordingly, any means for target DNA sequence selection can be used in the methods described herein. A target site generally has a length of at least 9 nucleotides and, accordingly, is bound by a zinc finger binding domain comprising at least three zinc fingers. However binding of, for example, a 4-finger binding domain to a 12-nucleotide target site, a 5-finger binding domain to a 15-nucleotide target site or a 6-finger binding domain to an 18-nucleotide target site, is also possible. As will be apparent, binding of larger binding domains (e.g., 7-, 8-, 9-finger and more) to longer target sites is also possible.

In some embodiments, the zinc finger binding domains bind to a target DNA sequence that is at least 90% identical to a target DNA sequence (e.g., epitope-encoding) within a target locus of a target CD117 gene. In some embodiments, the zinc finger binding domains bind to a target DNA sequence that is at least 95%, 96%, 97%, 98%, or 99% identical to a target DNA sequence within a target locus of a target gene. In some embodiments, the zinc finger binding domains bind to a target DNA sequence that is 100% identical to a target DNA sequence within a target locus of a target gene.

The enzymatic domain portion of the zinc finger fusion proteins can be obtained from any endo- or exonuclease. Exemplary endonucleases from which an enzymatic domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., 51 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains. Exemplary restriction endonucleases (restriction enzymes) suitable for use as an enzymatic domain of the ZFPs described herein are present in many species and are capable of sequence-specific binding to DNA (at a recognition site), and cleaving DNA at or near the site of binding. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the enzymatic domain from at least one Type IIS restriction enzyme and one or more zinc finger binding domains.

An exemplary Type IIS restriction enzyme, whose cleavage domain is separable from the binding domain, is Fok I. This particular enzyme is active as a dimer. Bitinaite et al. (1998) Proc. Natl. Acad. Sci. USA 95: 10,570-10,575. Thus, for targeted double-stranded DNA cleavage using zinc finger-Fok I fusions, two fusion proteins, each comprising a FokI enzymatic domain, can be used to reconstitute a catalytically active cleavage domain. Alternatively, a single polypeptide molecule containing a zinc finger binding domain and two FokI enzymatic domains can also be used. Exemplary ZFPs comprising FokI enzymatic domains are described in U.S. Pat. No. 9,782,437.

TALEN-based systems comprise a protein comprising a TAL effector DNA binding domain and an enzymatic domain. They are made by fusing a TAL effector DNA-binding domain to a DNA cleavage domain (a nuclease which cuts DNA strands). The FokI restriction enzyme described above is an exemplary enzymatic domain suitable for use in TALEN-based gene regulating systems.

TAL effectors are proteins that are secreted by Xanthomonas bacteria via their type III secretion system when they infect plants. The DNA binding domain contains a repeated, highly conserved, 33-34 amino acid sequence with divergent 12th and 13th amino acids. These two positions, referred to as the Repeat Variable Diresidue (RVD), are highly variable and strongly correlated with specific nucleotide recognition. Therefore, the TAL effector domains can be engineered to bind specific target DNA sequences by selecting a combination of repeat segments containing the appropriate RVDs. The nucleic acid specificity for RVD combinations is as follows: HD targets cytosine, NI targets adenenine, NG targets thymine, and NN targets guanine (though, in some embodiments, NN can also bind adenenine with lower specificity).

In some embodiments, the TAL effector domains bind to a target DNA sequence that is at least 90% identical to a target DNA sequence (e.g., epitope-enoding) within a target locus of a CD117 gene. In some embodiments, the TAL effector domains bind to a target DNA sequence that is at least 95%, 96%, 97%, 98%, or 99% identical to a target DNA sequence within a target locus of a target gene. In some embodiments, the TAL effector domains bind to a target DNA sequence that is 100% identical to a target DNA sequence within a target locus of a target gene. Methods and compositions for assembling the TAL-effector repeats are known in the art. See e.g., Cermak et al, Nucleic Acids Research, 39:12, 2011, e82. Plasmids for constructions of the TAL-effector repeats are commercially available from Addgene.

In some embodiments, the gene-editing system is a combination gene-regulating system comprising a site-directed modifying polypeptide and a nucleic acid guide molecule. Herein, a “site-directed modifying polypeptide” refers to a polypeptide that binds to a nucleic acid guide molecule, is targeted to a target nucleic acid sequence, such as, for example, a DNA sequence, by the nucleic acid guide molecule to which it is bound, and modifies the target DNA sequence (e.g., cleavage, mutation, or methylation of target DNA). A site-directed modifying polypeptide comprises two portions, a portion that binds the nucleic acid guide and an activity portion. In some embodiments, a site-directed modifying polypeptide comprises an activity portion that exhibits site-directed enzymatic activity (e.g., DNA methylation, DNA cleavage, histone acetylation, histone methylation, etc.), wherein the site of enzymatic activity is determined by the guide nucleic acid.

In particular embodiments, the nucleic acid guide comprises two portions: a first portion that is complementary to, and capable of binding with, an endogenous target DNA sequence (referred to herein as a “DNA-binding segment”), and a second portion that is capable of interacting with the site-directed modifying polypeptide (referred to herein as a “protein-binding segment”). In some embodiments, the DNA-binding segment and protein-binding segment of a nucleic acid guide are comprised within a single polynucleotide molecule. In some embodiments, the DNA-binding segment and protein-binding segment of a nucleic acid guide are each comprised within separate polynucleotide molecules, such that the nucleic acid guide comprises two polynucleotide molecules that associate with each other to form the functional guide.

The nucleic acid guide mediates the target specificity of the combined protein/nucleic gene regulating systems by specifically hybridizing with a target DNA sequence comprised within the DNA sequence of a target gene. Reference herein to a target gene encompasses the full-length DNA sequence for that particular gene and a full-length DNA sequence for a particular target gene will comprise a plurality of target genetic loci, which refer to portions of a particular target gene sequence (e.g., an exon or an intron). Within each target genetic loci are shorter stretches of DNA sequences referred to herein as “target DNA sequences” or “target sequences” that can be modified by the gene-regulating systems described herein. Further, each target genetic loci comprises a “target modification site,” which refers to the precise location of the modification induced by the gene-regulating system (e.g., the location of an insertion, a deletion, or mutation, the location of a DNA break, or the location of an epigenetic modification). The gene-regulating systems described herein may comprise a single nucleic acid guide, or may comprise a plurality of nucleic acid guides (e.g., 2, 3, 4, 5, 6, 7, 8, 9, 10, or more nucleic acid guides).

The CRISPR/Cas systems described below are exemplary embodiments of a combination protein/nucleic acid system.

In some embodiments, the gene editing systems described herein are CRISPR (Clustered Regularly Interspaced Short Palindromic Repeats)/Cas (CRISPR Associated) nuclease systems. In such embodiments, the site-directed modifying polypeptide is a CRISPR-associated endonuclease (a “Cas” endonuclease) and the nucleic acid guide molecule is a guide RNA (gRNA).

A Cas polypeptide refers to a polypeptide that can interact with a gRNA molecule and, in concert with the gRNA molecule, homes or localizes to a target DNA sequence and includes naturally occurring Cas proteins and engineered, altered, or otherwise modified Cas proteins that differ by one or more amino acid residues from a naturally-occurring Cas sequence.

In some embodiments, the Cas protein is a Cas9 protein. Cas9 is a multi-domain enzyme that uses an HNH nuclease domain to cleave the target strand of DNA and a RuvC-like domain to cleave the non-target strand. In some embodiments, mutants of Cas9 can be generated by selective domain inactivation enabling the conversion of WT Cas9 into an enzymatically inactive mutant (e.g., dCas9), which is unable to cleave DNA, or a nickase mutant, which is able to produce single-stranded DNA breaks by cleaving one or the other of the target or non-target strand.

A guide RNA (gRNA) typically comprises two segments, a DNA-binding segment and a protein-binding segment. In some embodiments, the protein-binding segment of a gRNA is comprised in one RNA molecule and the DNA-binding segment is comprised in another separate RNA molecule. Such embodiments are referred to herein as “double-molecule gRNAs” or “two-molecule gRNA” or “dual gRNAs.” In some embodiments, the gRNA is a single RNA molecule and is referred to herein as a “single-guide RNA” or an “sgRNA.” The term “guide RNA” or “gRNA” is inclusive, referring both to two-molecule guide RNAs and sgRNAs.

The protein-binding segment of a gRNA typically comprises, in part, two complementary stretches of nucleotides that hybridize to one another to form a double stranded RNA duplex (dsRNA duplex), which facilitates binding to the Cas protein.

The DNA-binding segment (or “DNA-binding sequence”) of a gRNA comprises a nucleotide sequence that is complementary to and capable of binding to a specific sequence target DNA sequence or RNA sequence. The protein-binding segment of the gRNA interacts with a Cas polypeptide and the interaction of the gRNA molecule and site-directed modifying polypeptide results in Cas binding to the endogenous DNA or RNA and produces one or more modifications within or around the target DNA sequence. The precise location of the target modification site is determined by both (i) base-pairing complementarity between the gRNA and the target DNA or RNA sequence; and (ii) the location of a short motif, referred to as the protospacer adjacent motif (PAM), in the target DNA sequence. The PAM sequence is required for Cas binding to the target DNA sequence. A variety of PAM sequences are known in the art and are suitable for use with a particular Cas endonuclease (e.g., a Cas9 endonuclease) are known in the art (See e.g., Nat Methods. 2013 November; 10(11): 1116-1121 and Sci Rep. 2014; 4: 5405). In some embodiments, the PAM sequence is located within 50 base pairs of the target modification site. In some embodiments, the PAM sequence is located within 10 base pairs of the target modification site. The DNA or RNA sequences that can be targeted by this method are limited only by the relative distance of the PAM sequence to the target modification site and the presence of a unique 20 base pair sequence to mediate sequence-specific, gRNA-mediated Cas binding. In some embodiments, the target modification site is located at the 5′ terminus of the target locus. In some embodiments, the target modification site is located at the 3′ end of the target locus. In some embodiments, the target modification site is located within an intron or an exon of the target locus.

In particular embodiments, the guide RNA binds to a CD117 polynucleotide sequence and includes a region complementary to a target CD117 sequence. In certain embodiments, the guide RNA targets or binds a region of CD117 polynucleotide sequence that encodes one of the following amino acid residues: E73, D121, R122, S123, Y125, K203, Y259, S261, W262, Y269, or R271. In some embodiments, the present disclosure provides a polynucleotide encoding a gRNA. In some embodiments, a gRNA-encoding nucleic acid is comprised in an expression vector, e.g., a recombinant expression vector. In some embodiments, the present disclosure provides a polynucleotide encoding a site-directed modifying polypeptide. In some embodiments, the polynucleotide encoding a site-directed modifying polypeptide is comprised in an expression vector, e.g., a recombinant expression vector.

In some embodiments, the site-directed modifying polypeptide is a Cas protein, e.g., a Cas9 protein. Cas molecules of a variety of species can be used in the methods and compositions described herein, including Cas molecules derived from S. pyogenes, S. aureus, N. meningitidis, S. thermophiles, etc. In some embodiments, the Cas protein is a Cas9 protein or a Cas9 ortholog and is selected from the group consisting of SpCas9, SpCas9-HF1, SpCas9-HF2, SpCas9-HF3, SpCas9-HF4, SaCas9, FnCpf, FnCas9, eSpCas9, and NmeCas9. In some embodiments, the Cas9 protein is a naturally-occurring Cas9 protein. Exemplary naturally occurring Cas9 molecules are described in Chylinski et al., RNA Biology 2013 10:5, 727-737.

In some embodiments, a Cas9 protein comprises an amino acid sequence having at least 60%, 65%, 70%, 75%, 80%, 85%, 90%, 95%, 96%, 97%, 98%, 99%, or 100% sequence identity to a Cas9 amino acid sequence described in Chylinski et al., RNA Biology 2013 10:5, 727-737; Hou et al., PNAS Early Edition 2013, 1-6).

In some embodiments, a Cas polypeptide comprises one or more of the following activities:
a) a nickase activity, i.e., the ability to cleave a single strand, e.g., the non-complementary strand or the complementary strand, of a nucleic acid molecule;
b) a double stranded nuclease activity, i.e., the ability to cleave both strands of a double stranded nucleic acid and create a double stranded break, which in an embodiment is the presence of two nickase activities;
c) an endonuclease activity;
d) an exonuclease activity; and/or
e) a helicase activity, i.e., the ability to unwind the helical structure of a double stranded nucleic acid.

In some embodiments, the Cas9 is a wildtype (WT) Cas9 protein or ortholog. WT Cas9 comprises two catalytically active domains (HNH and RuvC). Binding of WT Cas9 to DNA based on gRNA specificity results in double-stranded DNA breaks that can be repaired by non-homologous end joining (NHEJ) or homology-directed repair (HDR). In some embodiments, Cas9 is fused to heterologous proteins that recruit DNA-damage signaling proteins, exonucleases, or phosphatases to further increase the likelihood or the rate of repair of the target sequence by one repair mechanism or another. In some embodiments, a WT Cas9 is co-expressed with a nucleic acid repair template to facilitate the incorporation of an exogenous nucleic acid sequence by homology-directed repair.

In some embodiments, different Cas9 proteins (i.e., Cas9 proteins from various species) may be advantageous to use in the various provided methods in order to capitalize on various enzymatic characteristics of the different Cas9 proteins (e.g., for different PAM sequence preferences; for increased or decreased enzymatic activity; for an increased or decreased level of cellular toxicity; to change the balance between NHEJ, homology-directed repair, single strand breaks, double strand breaks, etc.).

In some embodiments, the Cas polypeptides are engineered to alter one or more properties of the Cas polypeptide. For example, in some embodiments, the Cas polypeptide comprises altered enzymatic properties, e.g., altered nuclease activity, (as compared with a naturally occurring or other reference Cas molecule) or altered helicase activity.

The present disclosure provides guide RNAs (gRNAs) that direct a site-directed modifying polypeptide to a specific target DNA sequence. A gRNA comprises a DNA-targeting segment and protein-binding segment. The DNA-targeting segment of a gRNA comprises a nucleotide sequence that is complementary to a sequence in the target DNA sequence. As such, the DNA-targeting segment of a gRNA interacts with a target DNA in a sequence-specific manner via hybridization (i.e., base pairing), and the nucleotide sequence of the DNA-targeting segment determines the location within the target DNA that the gRNA will bind. The DNA-targeting segment of a gRNA can be modified (e.g., by genetic engineering) to hybridize to any desired sequence within a target DNA sequence.

The protein-binding segment of a guide RNA interacts with a site-directed modifying polypeptide (e.g. a Cas9 protein) to form a complex. The guide RNA guides the bound polypeptide to a specific nucleotide sequence within target DNA via the above-described DNA-targeting segment. The protein-binding segment of a guide RNA comprises two stretches of nucleotides that are complementary to one another and which form a double stranded RNA duplex.

In some embodiments, a gRNA comprises two separate RNA molecules. In such embodiments, each of the two RNA molecules comprises a stretch of nucleotides that are complementary to one another such that the complementary nucleotides of the two RNA molecules hybridize to form the double-stranded RNA duplex of the protein-binding segment. In some embodiments, a gRNA comprises a single RNA molecule (sgRNA).

The specificity of a gRNA for a target loci is mediated by the sequence of the DNA-binding segment, which comprises about 20 nucleotides that are complementary to a target DNA sequence within the target locus. In some embodiments, the corresponding target DNA sequence is approximately 20 nucleotides in length. In some embodiments, the DNA-binding segments of the gRNA sequences of the present invention are at least 90% complementary to a target DNA sequence within a target locus. In some embodiments, the DNA-binding segments of the gRNA sequences of the present disclosure are at least 95%, 96%, 97%, 98%, or 99% complementary to a target DNA sequence within a target locus, e.g., CD117. In some embodiments, the DNA-binding segments of the gRNA sequences of the present invention are 100% complementary to a target DNA sequence within a target locus.

In some embodiments, the DNA-binding segments of the gRNA sequences bind to a target DNA sequence that is at least 90% identical to a target DNA sequence within a target locus of a CD117 gene. In some embodiments, the DNA-binding segments of the gRNA sequences bind to a target DNA sequence that is at least 95%, 96%, 97%, 98%, or 99% identical to a target DNA sequence within a target locus of a target gene. In some embodiments, the DNA-binding segments of the gRNA sequences bind to a target DNA sequence that is 100% identical to a target DNA sequence within a target locus of a target gene.

In some embodiments, the DNA-binding segments of the gRNA sequences described herein are designed to minimize off-target binding using algorithms known in the art (e.g., Cas-OFF finder) to identify target sequences that are unique to a particular target locus or target gene. In some embodiments, the gRNAs described herein can comprise one or more modified nucleosides or nucleotides which introduce stability toward nucleases. In such embodiments, these modified gRNAs may elicit a reduced innate immune as compared to a non-modified gRNA. The term “innate immune response” includes a cellular response to exogenous nucleic acids, including single stranded nucleic acids, generally of viral or bacterial origin, which involves the induction of cytokine expression and release, particularly the interferons, and cell death.

In some embodiments, the gRNAs described herein are modified at or near the 5′ end (e.g., within 1-10, 1-5, or 1-2 nucleotides of their 5′ end). In some embodiments, the 5′ end of a gRNA is modified by the inclusion of a eukaryotic mRNA cap structure or cap analog (e.g., a G(5′)ppp(5′)G cap analog, a m7G(5′)ppp(5′)G cap analog, or a 3′-0-Me-m7G(5′)ppp(5′)G anti reverse cap analog (ARCA)). In some embodiments, an in vitro transcribed gRNA is modified by treatment with a phosphatase (e.g., calf intestinal alkaline phosphatase) to remove the 5′ triphosphate group. In some embodiments, a gRNA comprises a modification at or near its 3′ end (e.g., within 1-10, 1-5, or 1-2 nucleotides of its 3′ end). For example, in some embodiments, the 3′ end of a gRNA is modified by the addition of one or more (e.g., 25-200) adenine (A) residues.

In some embodiments, modified nucleosides and modified nucleotides can be present in a gRNA, but also may be present in other gene-regulating systems, e.g., mRNA, RNAi, or siRNA-based systems. In some embodiments, modified nucleosides and nucleotides can include one or more of.
a) alteration, e.g., replacement, of one or both of the non-linking phosphate oxygens and/or of one or more of the linking phosphate oxygens in the phosphodiester backbone linkage;
b) alteration, e.g., replacement, of a constituent of the ribose sugar, e.g., of the 2′ hydroxyl on the ribose sugar;
c) wholesale replacement of the phosphate moiety with “dephospho” linkers;
d) modification or replacement of a naturally occurring nucleobase;
e) replacement or modification of the ribose-phosphate backbone;
f) modification of the 3′ end or 5′ end of the oligonucleotide, e.g., removal, modification or replacement of a terminal phosphate group or conjugation of a moiety; and
g) modification of the sugar.

In some embodiments, the modifications listed above can be combined to provide modified nucleosides and nucleotides that can have two, three, four, or more modifications. For example, in some embodiments, a modified nucleoside or nucleotide can have a modified sugar and a modified nucleobase. In some embodiments, every base of a gRNA is modified. In some embodiments, each of the phosphate groups of a gRNA molecule are replaced with phosphorothioate groups.

In some embodiments, a software tool can be used to optimize the choice of gRNA within a user's target sequence, e.g., to minimize total off-target activity across the genome. Off target activity may be other than cleavage. For example, for each possible gRNA choice using S. pyogenes Cas9, software tools can identify all potential off-target sequences (preceding either NAG or NGG PAMs) across the genome that contain up to a certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible gRNA can then be ranked according to its total predicted off-target cleavage; the top-ranked gRNAs represent those that are likely to have the greatest on-target and the least off-target cleavage. Other functions, e.g., automated reagent design for gRNA vector construction, primer design for the on-target Surveyor assay, and primer design for high-throughput detection and quantification of off-target cleavage via next-generation sequencing, can also be included in the tool.

Additional information regarding CRISPR-Cas systems and components thereof are described in, U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641 and applications related thereto; and WO2014/018423, WO2014/093595, WO2014/093622, WO2014/093635, WO2014/093655, WO2014/093661, WO2014/093694, WO2014/093701, WO2014/093709, WO2014/093712, WO2014/093718, WO2014/145599, WO2014/204723, WO2014/204724, WO2014/204725, WO2014/204726, WO2014/204727, WO2014/204728, WO2014/204729, WO2015/065964, WO2015/089351, WO2015/089354, WO2015/089364, WO2015/089419, WO2015/089427, WO2015/089462, WO2015/089465, WO2015/089473 and WO2015/089486, WO2016205711, WO2017/106657, WO2017/127807 and applications related thereto.

In certain embodiments, the gene editing methods comprise or consist of base editing methods. Various base editing methods are well known in the art and may be adapted to edit any target nucleic acid sequence within a cell. Base editing activity involves chemically altering a base within a polynucleotide, e.g., converting a first base to a second base. In one embodiment, the base editing activity is cytidine deaminase activity, e.g., converting target C⋅G to T⋅A. In another embodiment, the base editing activity is adenosine or adenine deaminase activity, e.g., converting A⋅T to G⋅C. In another embodiment, the base editing activity is cytidine deaminase activity, e.g., converting target C⋅G to T⋅A and adenosine or adenine deaminase activity, e.g., converting A⋅T to G⋅C. In some embodiments, the base editing methods comprise single nucleotide base editing, such as nucleotide deamination, i.e., A→G or C→T. Base editing systems may edit genomic DNA or transcribed RNA.

A variety of base editing methods are known and used in the art, including but not limited to those disclosed in the references cited herein. Adenosine and cytidine base editors that may be used include, but are not limited to, base editors described in Antoniou P. et al., Base and Prime Editing Technologies for Blood Disorders, Front. Genome Ed., 28 Jan. 2021. In some embodiments, base editing methods comprise C→G conversion as described in Kurt, I. C. et al. CRISPR C-to-G base editors for inducing targeted DNA transversions in human cells. Nat. Biotechnol. (2020). In some embodiments, dual editors facilitate simultaneous C→T and A→G conversion as described in Zhao, D. et al. New base editors change C to A in bacteria and C to G in mammalian cells. Nat. Biotechnol. (2020).

A base editor system generally refers to a system for editing a nucleobase of a target nucleotide sequence. In certain embodiments, a base editor (BE) system comprises: (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises: (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine base editor (CBE).

Cas9 or Cas9 domain refers to an RNA guided nuclease comprising a Cas9 protein, or a fragment or variant thereof (e.g., a protein comprising an active, inactive, or partially active DNA cleavage domain of Cas9, and/or the gRNA binding domain of Cas9). A variety of different Cas9 proteins, and fragments and variants thereof, are known and available in the art.

A guide polynucleotide is a polynucleotide that is specific for a target sequence (e.g., specifically hybridizes to a target polynucleotide sequence, such as a CD117 gene or mRNA) and can form a complex with a polynucleotide programmable nucleotide binding domain protein (e.g., Cas9 or Cpfl). In certain embodiments, the guide polynucleotide is a guide RNA (gRNA). gRNAs can exist as a complex of two or more RNAs, or as a single RNA molecule. gRNA is used to refer to guide RNAs that exist as either single molecules or as a complex of two or more molecules gRNAs, and gRNAs that exist as a single RNA molecule may be referred to as single-guide RNAs (sgRNAs). gRNAs that exist as single RNA species may comprise two domains: (1) a domain that shares homology to a target nucleic acid, and thus directs binding of a Cas9 complex to the target nucleic acid; and (2) a domain that binds a Cas9 protein. In some embodiments, domain (2) is a sequence known as a tracrRNA, which comprises a stem-loop structure. For example, in some embodiments, domain (2) is identical or homologous to a tracrRNA as provided in Jinek et al., Science 337:816-821(2012). Other examples of gRNAs (e.g., those including domain 2) are described, e.g., in US20160208288, entitled “Switchable Cas9 Nucleases and Uses Thereof,” and U.S. Pat. No. 9,737,604, entitled “Delivery System For Functional Nucleases.” In some embodiments, a gRNA comprises two or more of domains (1) and (2), which may be referred to as an extended gRNA. An extended gRNA will bind two or more Cas9 proteins and bind a target nucleic acid at two or more distinct regions. The gRNA comprises a nucleotide sequence that complements a target site, which mediates binding of the nuclease/RNA complex to the target site, providing the sequence specificity of the nuclease: RNA complex.

In some embodiments, the base editing method, e.g., a single nucleotide base editing method, targets a polynucleotide encoding a CD117 polypeptide. An illustrative CD117 polynucleotide sequence follows:
>NM_000222.2 Homo sapiens KIT proto-oncogene, receptor tyrosine kinase (KIT), transcript variant 1 mRNA:

(SEQ ID NO: 18)
TCTGGGGGCTCGGCTTTGCCGCGCTCGCTGCACTTGGGCGAGAGCTGGAACGTG
GACCAGAGCTCGGATCCCATCGCAGCTACCGCGATGAGAGGCGCTCGCGGCGC
CTGGGATTTTCTCTGCGTTCTGCTCCTACTGCTTCGCGTCCAGACAGGCTCTTCT
CAACCATCTGTGAGTCCAGGGGAACCGTCTCCACCATCCATCCATCCAGGAAAA
TCAGACTTAATAGTCCGCGTGGGCGACGAGATTAGGCTGTTATGCACTGATCCG
GGCTTTGTCAAATGGACTTTTGAGATCCTGGATGAAACGAATGAGAATAAGCAG
AATGAATGGATCACGGAAAAGGCAGAAGCCACCAACACCGGCAAATACACGTG
CACCAACAAACACGGCTTAAGCAATTCCATTTATGTGTTTGTTAGAGATCCTGC
CAAGCTTTTCCTTGTTGACCGCTCCTTGTATGGGAAAGAAGACAACGACACGCT
GGTCCGCTGTCCTCTCACAGACCCAGAAGTGACCAATTATTCCCTCAAGGGGTG
CCAGGGGAAGCCTCTTCCCAAGGACTTGAGGTTTATTCCTGACCCCAAGGCGGG
CATCATGATCAAAAGTGTGAAACGCGCCTACCATCGGCTCTGTCTGCATTGTTCT
GTGGACCAGGAGGGCAAGTCAGTGCTGTCGGAAAAATTCATCCTGAAAGTGAG
GCCAGCCTTCAAAGCTGTGCCTGTTGTGTCTGTGTCCAAAGCAAGCTATCTTCTT
AGGGAAGGGGAAGAATTCACAGTGACGTGCACAATAAAAGATGTGTCTAGTTC
TGTGTACTCAACGTGGAAAAGAGAAAACAGTCAGACTAAACTACAGGAGAAAT
ATAATAGCTGGCATCACGGTGACTTCAATTATGAACGTCAGGCAACGTTGACTA
TCAGTTCAGCGAGAGTTAATGATTCTGGAGTGTTCATGTGTTATGCCAATAATA
CTTTTGGATCAGCAAATGTCACAACAACCTTGGAAGTAGTAGATAAAGGATTCA
TTAATATCTTCCCCATGATAAACACTACAGTATTTGTAAACGATGGAGAAAATG
TAGATTTGATTGTTGAATATGAAGCATTCCCCAAACCTGAACACCAGCAGTGGA
TCTATATGAACAGAACCTTCACTGATAAATGGGAAGATTATCCCAAGTCTGAGA
ATGAAAGTAATATCAGATACGTAAGTGAACTTCATCTAACGAGATTAAAAGGC
ACCGAAGGAGGCACTTACACATTCCTAGTGTCCAATTCTGACGTCAATGCTGCC
ATAGCATTTAATGTTTATGTGAATACAAAACCAGAAATCCTGACTTACGACAGG
CTCGTGAATGGCATGCTCCAATGTGTGGCAGCAGGATTCCCAGAGCCCACAAT
AGATTGGTATTTTTGTCCAGGAACTGAGCAGAGATGCTCTGCTTCTGTACTGCCA
GTGGATGTGCAGACACTAAACTCATCTGGGCCACCGTTTGGAAAGCTAGTGGTT
CAGAGTTCTATAGATTCTAGTGCATTCAAGCACAATGGCACGGTTGAATGTAAG
GCTTACAACGATGTGGGCAAGACTTCTGCCTATTTTAACTTTGCATTTAAAGGTA
ACAACAAAGAGCAAATCCATCCCCACACCCTGTTCACTCCTTTGCTGATTGGTTT
CGTAATCGTAGCTGGCATGATGTGCATTATTGTGATGATTCTGACCTACAAATAT
TTACAGAAACCCATGTATGAAGTACAGTGGAAGGTTGTTGAGGAGATAAATGG
AAACAATTATGTTTACATAGACCCAACACAACTTCCTTATGATCACAAATGGGA
GTTTCCCAGAAACAGGCTGAGTTTTGGGAAAACCCTGGGTGCTGGAGCTTTCGG
GAAGGTTGTTGAGGCAACTGCTTATGGCTTAATTAAGTCAGATGCGGCCATGAC
TGTCGCTGTAAAGATGCTCAAGCCGAGTGCCCATTTGACAGAACGGGAAGCCCT
CATGTCTGAACTCAAAGTCCTGAGTTACCTTGGTAATCACATGAATATTGTGAA
TCTACTTGGAGCCTGCACCATTGGAGGGCCCACCCTGGTCATTACAGAATATTG
TTGCTATGGTGATCTTTTGAATTTTTTGAGAAGAAAACGTGATTCATTTATTTGT
TCAAAGCAGGAAGATCATGCAGAAGCTGCACTTTATAAGAATCTTCTGCATTCA
AAGGAGTCTTCCTGCAGCGATAGTACTAATGAGTACATGGACATGAAACCTGGA
GTTTCTTATGTTGTCCCAACCAAGGCCGACAAAAGGAGATCTGTGAGAATAGGC
TCATACATAGAAAGAGATGTGACTCCCGCCATCATGGAGGATGACGAGTTGGCC
CTAGACTTAGAAGACTTGCTGAGCTTTTCTTACCAGGTGGCAAAGGGCATGGCT
TTCCTCGCCTCCAAGAATTGTATTCACAGAGACTTGGCAGCCAGAAATATCCTC
CTTACTCATGGTCGGATCACAAAGATTTGTGATTTTGGTCTAGCCAGAGACATC
AAGAATGATTCTAATTATGTGGTTAAAGGAAACGCTCGACTACCTGTGAAGTGG
ATGGCACCTGAAAGCATTTTCAACTGTGTATACACGTTTGAAAGTGACGTCTGG
TCCTATGGGATTTTTCTTTGGGAGCTGTTCTCTTTAGGAAGCAGCCCCTATCCTG
GAATGCCGGTCGATTCTAAGTTCTACAAGATGATCAAGGAAGGCTTCCGGATGC
TCAGCCCTGAACACGCACCTGCTGAAATGTATGACATAATGAAGACTTGCTGGG
ATGCAGATCCCCTAAAAAGACCAACATTCAAGCAAATTGTTCAGCTAATTGAGA
AGCAGATTTCAGAGAGCACCAATCATATTTACTCCAACTTAGCAAACTGCAGCC
CCAACCGACAGAAGCCCGTGGTAGACCATTCTGTGCGGATCAATTCTGTCGGCA
GCACCGCTTCCTCCTCCCAGCCTCTGCTTGTGCACGACGATGTCTGAGCAGAATC
AGTGTTTGGGTCACCCCTCCAGGAATGATCTCTTCTTTTGGCTTCCATGATGGTT
ATTTTCTTTTCTTTCAACTTGCATCCAACTCCAGGATAGTGGGCACCCCACTGCA
ATCCTGTCTTTCTGAGCACACTTTAGTGGCCGATGATTTTTGTCATCAGCCACCA
TCCTATTGCAAAGGTTCCAACTGTATATATTCCCAATAGCAACGTAGCTTCTACC
ATGAACAGAAAACATTCTGATTTGGAAAAAGAGAGGGAGGTATGGACTGGGGG
CCAGAGTCCTTTCCAAGGCTTCTCCAATTCTGCCCAAAAATATGGTTGATAGTTT
ACCTGAATAAATGGTAGTAATCACAGTTGGCCTTCAGAACCATCCATAGTAGTA
TGATGATACAAGATTAGAAGCTGAAAACCTAAGTCCTTTATGTGGAAAACAGA
ACATCATTAGAACAAAGGACAGAGTATGAACACCTGGGCTTAAGAAATCTAGT
ATTTCATGCTGGGAATGAGACATAGGCCATGAAAAAAATGATCCCCAAGTGTG
AACAAAAGATGCTCTTCTGTGGACCACTGCATGAGCTTTTATACTACCGACCTG
GTTTTTAAATAGAGTTTGCTATTAGAGCATTGAATTGGAGAGAAGGCCTCCCTA
GCCAGCACTTGTATATACGCATCTATAAATTGTCCGTGTTCATACATTTGAGGGG
AAAACACCATAAGGTTTCGTTTCTGTATACAACCCTGGCATTATGTCCACTGTGT
ATAGAAGTAGATTAAGAGCCATATAAGTTTGAAGGAAACAGTTAATACCATTTT
TTAAGGAAACAATATAACCACAAAGCACAGTTTGAACAAAATCTCCTCTTTTAG
CTGATGAACTTATTCTGTAGATTCTGTGGAACAAGCCTATCAGCTTCAGAATGG
CATTGTACTCAATGGATTTGATGCTGTTTGACAAAGTTACTGATTCACTGCATGG
CTCCCACAGGAGTGGGAAAACACTGCCATCTTAGTTTGGATTCTTATGTAGCAG
GAAATAAAGTATAGGTTTAGCCTCCTTCGCAGGCATGTCCTGGACACCGGGCCA
GTATCTATATATGTGTATGTACGTTTGTATGTGTGTAGACAAATATTTGGAGGGG
TATTTTTGCCCTGAGTCCAAGAGGGTCCTTTAGTACCTGAAAAGTAACTTGGCTT
TCATTATTAGTACTGCTCTTGTTTCTTTTCACATAGCTGTCTAGAGTAGCTTACCA
GAAGCTTCCATAGTGGTGCAGAGGAAGTGGAAGGCATCAGTCCCTATGTATTTG
CAGTTCACCTGCACTTAAGGCACTCTGTTATTTAGACTCATCTTACTGTACCTGT
TCCTTAGACCTTCCATAATGCTACTGTCTCACTGAAACATTTAAATTTTACCCTT
TAGACTGTAGCCTGGATATTATTCTTGTAGTTTACCTCTTTAAAAACAAAACAAA
ACAAAACAAAAAACTCCCCTTCCTCACTGCCCAATATAAAAGGCAAATGTGTAC
ATGGCAGAGTTTGTGTGTTGTCTTGAAAGATTCAGGTATGTTGCCTTTATGGTTT
CCCCCTTCTACATTTCTTAGACTACATTTAGAGAACTGTGGCCGTTATCTGGAAG
TAACCATTTGCACTGGAGTTCTATGCTCTCGCACCTTTCCAAAGTTAACAGATTT
TGGGGTTGTGTTGTCACCCAAGAGATTGTTGTTTGCCATACTTTGTCTGAAAAAT
TCCTTTGTGTTTCTATTGACTTCAATGATAGTAAGAAAAGTGGTTGTTAGTTATA
GATGTCTAGGTACTTCAGGGGCACTTCATTGAGAGTTTTGTCTTGGATATTCTTG
AAAGTTTATATTTTTATAATTTTTTCTTACATCAGATGTTTCTTTGCAGTGGCTTA
ATGTTTGAAATTATTTTGTGGCTTTTTTTGTAAATATTGAAATGTAGCAATAATG
TCTTTTGAATATTCCCAAGCCCATGAGTCCTTGAAAATATTTTTTATATATACAG
TAACTTTATGTGTAAATACATAAGCGGCGTAAGTTTAAAGGATGTTGGTGTTCC
ACGTGTTTTATTCCTGTATGTTGTCCAATTGTTGACAGTTCTGAAGAATTCTAAT
AAAATGTACATATATAAATCAAAAAAAAAAAAAAAA.

In particular embodiments, the guide RNA binds to a CD117 polynucleotide sequence and includes a region complementary to a target CD117 sequence. In certain embodiments, the guide RNA targets or binds a region of CD117 polynucleotide sequence that encodes one of the following amino acid residues: E73, D121, R122, S123, Y125, K203, Y259, S261, W262, Y269, or R271.

One example of base editing methods, systems, and components thereof that may be used according to the methods and compositions disclosed herein is described in PCT Application Publication No. WO2021041945. In some embodiments, the base editing method comprises use of a modified CRISPR protein, bound to a guide RNA, and a base editing enzyme, such as a deaminase, wherein the modified CRISPR protein does not cause a double-stranded break. In some embodiments, the modified CRISPR protein is a nucleobase editor polypeptide or nucleic acid programmable-DNA binding protein (napDNAbp), as disclosed in PCT Application Publication Nos. WO2021041945 or WO2021163587 In some embodiments, the method of base editing a polynucleotide encoding a CD117 polypeptide comprises expressing in a cell a nucleobase editor polypeptide, wherein the nucleobase editor polypeptide comprises a napDNAbp and a deaminase, and contacting the cell with a guide RNA capable of targeting the polynucleotide encoding a CD117 polypeptide.

In other embodiments, base editing may refer to RNA base editing methods, e.g., as described in Porto E. et al. Base editing: advances and therapeutic opportunities, Nature Reviews Drug Discovery volume 19, pages 839-859 (2020).

In particular embodiments, any of the gene editing, including base editing methods disclosed herein or known in the art may be used to modify one or more amino acids within an epitope of wild type human CD117 bound by an anti-CD117 antibody, optionally wherein the epitope comprises one or more of the following amino acids present in the wild type human CD117: E73, D121, R122, S123, Y125, K203, Y259, S261, W262, Y269, or R271, including but not limited to any of these recited amino acid residues. In certain embodiments, the method introduces a A→G or C→T mutation into one or both alleles of the CD117 gene, which results in the gene encoding a different amino acid by the codon that was mutated.

In certain embodiments, the disclosure provides a modified cell, e.g., HSPC or HSC, that comprises one or more components of a gene editing, e.g., base editing, system disclosed herein. In particular embodiments, the one or more component comprises a nucleic acid that binds to a CD117 gene or encoded mRNA, e.g., at a site to be modified to result in the encoding and/or expression of a modified CD117 disclosed herein, such as, e.g., a guide RNA. In particular embodiments, the guide RNA binds to a CD117 polynucleotide sequence and includes a region complementary to a target CD117 sequence. In certain embodiments, the guide RNA targets or binds a region of CD117 polynucleotide sequence that encodes one of the following amino acid residues: E73, D121, R122, S123, Y125, K203, Y259, S261, W262, Y269, or R271. In particular embodiments, the one or more component comprises a base editing enzyme, e.g., any of those disclosed herein or in references cited herein.

In particular embodiments, a modified cell expressing a modified CD117 polypeptide is not substantially inhibited, eliminated, or killed by monoclonal antibodies (mAbs) that bind endogenous or wild-type cell-surface CD117 and inhibit proliferation of or kill a cell expressing only the wild-type CD117 and not a modified CD117 polypeptide disclosed herein. In certain embodiments, proliferation of the modified cell expressing the modified CD117 polypeptide is inhibited by less than 50%, less than 40%, less than 30%, less than 20%, or less than 10%, as compared to proliferation of the same cell type that is not modified, e.g., only expresses wild-type CD117.

Compositions and methods disclosed herein may be applicable to any anti-CD117 antibody, particularly monoclonal anti-human CD117 antibodies. Illustrative CD117 signaling antibodies include, but are not limited to, SR-1, JSP191, 8D7, K45, 104D2, CK6, YB5.B8, AF-2-1, AF11, AF12, AF112, AF-3, AF-1-1, NF, NF-2-1, NF11, NF12, NF112, NF-3, HF11, HF12, and HF112. A number of antibodies contemplated by the disclosure that specifically bind human CD117 are known in the art and commercially available, including without limitation SR1, 2B8, ACK2, YB5-B8, 57A5, 104D2, etc. In certain embodiments, the anti-CD117 antibody is selected from the group consisting of: JSP191 (Jasper Therapeutics; Redwood City, CA); CDX-0159 (Celldex Therapeutics, Hampton, NJ); MGTA-117 (AB85) (Magenta Therapeutics, Cambridge, MA); CK6 (Magenta Therapeutics, Cambridge, MA); AB249 (Magenta Therapeutics, Cambridge, MA); and FSI-174 (Gilead, Foster City, CA). Antibodies from Magenta Therapeutics contemplated by the disclosure include but are not limited to those that are disclosed in US Patent Application Publication No. 20190153114, PCT Application Publication Nos. WO2019084064, WO2020/219748, and WO2020/219770. The FSI-174 antibody is disclosed in PCT application Publication No. WO2020/112687 and U.S. Patent Application Publication No. 20200165337. The disclosure includes but is not limited to any anti-CD117 antibodies and/or CDR sets disclosed in any of the patent application disclosed herein, which are all incorporated by reference in their entireties.

In certain embodiments, the anti-CD117 antibody binds to the extracellular region of CD117, i.e., amino acids 26-524. The sequence of this region is shown below:

(SEQ ID NO: 19)
QPSVSPGEPSPPSIHPGKSDLIVRVGDEIRLLCTDPGFVKWTFEILDET
NENKQNEWITEKAEATNTGKYTCTNKHGLSNSIYVFVRDPAKLFLVDRS
LYGKEDNDTLVRCPLTDPEVTNYSLKGCQGKPLPKDLRFIPDPKAGIMI
KSVKRAYHRLCLHCSVDQEGKSVLSEKFILKVRPAFKAVPVVSVSKASY
LLREGEEFTVTCTIKDVSSSVYSTWKRENSQTKLQEKYNSWHHGDFNYE
RQATLTISSARVNDSGVFMCYANNTFGSANVTTTLEVVDKGFINIFPMI
NTTVFVNDGENVDLIVEYEAFPKPEHQQWIYMNRTFTDKWEDYPKSENE
SNIRYVSELHLTRLKGTEGGTYTFLVSNSDVNAAIAFNVYVNTKPEILT
YDRLVNGMLQCVAAGFPEPTIDWYFCPGTEQRCSASVLPVDVQTLNSSG
PPFGKLVVQSSIDSSAFKHNGTVECKAYNDVGKTSAYFNFAFKGNNKEQ
IHPHTLFTP.

In particular embodiments, the antibody is a humanized form of SR1, which is a murine anti-CD117 antibody described in U.S. Pat. Nos. 5,919,911 and 5,489,516. The humanized form, JSP191 is disclosed in U.S. U.S. Pat. Nos. 7,915,391, 8,436,150, and 8,791,249. JSP191 is an aglycosylated IgG1 humanized antibody. JSP191 (formerly referred to as AMG191) is a humanized monoclonal antibody in clinical development as a conditioning agent to clear hematopoietic stem cells from bone marrow. JSP191 specifically binds to human CD117, a receptor for stem cell factor (SCF), which is expressed on the surface of hematopoietic stem and progenitor cells. JSP191 blocks SCF from binding to CD117 and disrupts critical survival signals, leading to the depletion of hematopoietic stem cells.

The sequences of the heavy chains and light chains of JSP191 are disclosed as SEQ ID NO: 4 from U.S. Pat. No. 8,436,150 and SEQ ID NO: 2 from U.S. Pat. No. 8,436,150,respectively. The sequences of the heavy and light chains of JSP191 are:

### Heavy Chain

(SEQ ID NO: 20)
MDWTWRVFCLLAVAPGAHSQVQLVQSGAEVKKPGASVKVSCKASGYTFT
SYNMHWVRQAPGQGLEWMGVIYSGNGDTSYNQKFKGRVTITADKSTSTA
YMELSSLRSEDTAVYYCARERDTRFGNWGQGTLVTVSSASTKGPSVFPL
APSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTC
PPCPAPELLGGPSVFLFPPKPKDTLMISRTPEVTCVVVDVSHEDPEVKF
NWYVDGVEVHNAKTKPREEQYQSTYRVVSVLTVLHQDWLNGKEYKCKVS
NKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNV
FSCSVMHEALHNHYTQKSLSLSPGK

### Light Chain

(SEQ ID NO: 21)
MVLQTQVFISLLLWISGAYGDIVMTQSPDSLAVSLGERATINCRASESV
DIYGNSFMHWYQQKPGQPPKLLIYLASNLESGVPDRFSGSGSGTDFTLT
ISSLQAEDVAVYYCQQNNEDPYTFGGGTKVEIKRTVAAPSVFIFPPSDE
QLKSGTASVVCLLNNFYPREAKVQWKVDNALQSGNSQESVTEQDSKDST
YSLSSTLTLSKADYEKHKVYACEVTHQGLSSPVTKSFNRGEC

In certain embodiments, the variable heavy domain of JSP191 comprises the following sequence:

(SEQ ID NO: 22)
QVQLVQSGAEVKKPGASVKVSCKASGYTFTSYNMHWVRQAPGQGLEWMG
VIYSGNGDTSYNQKFKGRVTITADKSTSTAYMELSSLRSEDTAVYYCAR
ERDTRFGNWGQGTLVTVSS.

In certain embodiments, the variable light chain domain of JSP191 comprises the following sequence:

(SEQ ID NO: 23)
DIVMTQSPDSLAVSLGERATINCRASESVDIYGNSFMHWYQQKPGQPPK
LLIYLASNLESGVPDRFSGSGSGTDFTLTISSLQAEDVAVYYCQQNNED
PYTFGGGTKVEIK .

The CDRs present in JSP191 are as follows:

VH CDR1 =
(SEQ ID NO: 24)
YNMH;
VH CDR2 =
(SEQ ID NO: 25)
IYSGNGDTSYNQKFKG;
VH CDR3 =
(SEQ ID NO: 26)
ERDTRFGN;
VL CDR1 =
(SEQ ID NO: 27)
RASESVDIYGNSFMH;
VL CDR2 =
(SEQ ID NO: 28)
LASNLES;
and
VL CDR3 =
(SEQ ID NO: 29)
QQNNEDPYT.

CDX-0159 is a humanized monoclonal antibody that specifically binds the receptor tyrosine kinase KIT with high specificity and potently inhibits its activity. CDX-0159 is designed to block KIT activation by disrupting both SCF binding and KIT dimerization. CDX-0159 and other anti-CD117 antibodies are described in U.S. Pat. No. 10,781,267, and in particular embodiments, an anti-CD117 disclosed herein comprises the CDRs of any of the antibodies disclosed therein. In certain embodiments, the anti-CD117 antibody comprises: (i) a light chain variable region (“VL”) comprising the amino acid sequence:

(SEQ ID NO: 30)
DIVMTQSPSX_{K1}LSASVGDRVTITCKASQNVRINVAWYQQKPGKAPKX_{K2}
LIYSASYRYSGVPDRFX_{K3}GSGSGTDFTLTISSLQX_{K4}EDFAX_{K5}YX_{K6}CQQ
YNSYPRTFGGGTKVEIK,

wherein X_{K1} is an amino acid with an aromatic or aliphatic hydroxyl side chain, X_{K2} is an amino acid with an aliphatic or aliphatic hydroxyl side chain, X_{K3} is an amino acid with an aliphatic hydroxyl side chain, X_{K4} is an amino acid with an aliphatic hydroxyl side chain or is P, X_{K5} is an amino acid with a charged or acidic side chain, and X_{K6} is an amino acid with an aromatic side chain; and (ii) a heavy chain variable region (“VH”) comprising the amino acid sequence:

(SEQ ID NO: 31)
QVQLVQSGAEXHIKKPGASVKX_{H2}SCKASGYTFTDYYINAVVX_{H3}QAPGK
GLEWIARIYPGSGNTYYNEKFKGRX_{H4}TX_{H5}TAX_{H6}KSTSTAYMX_{H7}LSSLR
SEDX_{H8}AVYFCARGVYYFDYWGQGTTVTVSS,

wherein X_{H1} is an amino acid with an aliphatic side chain, X_{H2} is an amino acid with an aliphatic side chain, X_{H3} is an amino acid with a polar or basic side chain, X_{H4} is an amino acid with an aliphatic side chain, X_{H5} is an amino acid with an aliphatic side chain, X_{H6} is an amino acid with an acidic side chain, X_{H7} is an amino acid with an acidic or amide derivative side chain, and X_{H8} is an amino acid with an aliphatic hydroxyl side chain. In specific aspects, described herein are antibodies (e.g., human or humanized antibodies), including antigen-binding fragments thereof, comprising: (i) VH CDRs of a VH domain comprising the amino acid sequence

(SEQ ID NO: 32)
QVQLKQSGAELVRPGASVKLSCKASGYTFTDYYINWVKQRPGQGLEWIA
RIYPGSGNTYYNEKFKGKATLTAEKSSSTAYMQLSSLTSEDSAVYFCAR
GVYYFDYWGQGTTLTVSS
or
(SEQ ID NO: 33)
QVQLKQSGAELVRPGASVKLSCKASGYTFTDYYINWVKQRPGQGLEWIA
RIYPGSGNTYYNEKFKGKATLTAEKSSSTAYMQLSSLTSEDSAVYFCAR
GVYYFDYWGQGTTLTVSA,
and
(ii) VL CDRs of a VL domain comprising the amino
acid sequence
(SEQ ID NO: 34)
DIVMTQSQKFMSTSVGDRVSVTCKASQNVRTNVAWYQQKPGQSPKALIY
SASYRYSGVPDRFTGSGSGTDFTLTISNVQSEDLADYFCQQYNSYPRTF
GGGTKLEIKR.

MGTA-117 (AB85) is a CD117-targeted antibody engineered for the transplant setting and conjugated to amanitin, which is being developed for patients undergoing immune reset through either autologous or allogeneic stem cell transplant. MGTA-117 depletes hematopoietic stem and progenitor cells, and this antibody and others contemplated by the disclosure are described in U.S. Application Publication No. 20200407440 and/or PCT Application Publication No. WO2019084064. Epitope analysis of AB85 binding to CD177 is described in PCT Application Publication No. WO2020219770, which identified the following two epitopes within CD117:

(SEQ ID NO: 35)
EKAEATNTGKYTCTNKHGLSNSIYVFVRDPA

(amino acids 60-90), and

(SEQ ID NO: 36)
RCPLTDPEVTNYSLKGCQGKP

(amino acids 100-130).

The sequences of the variable heavy chain and variable light chains of AB85 are disclosed as SEQ ID NO: 143 and SEQ ID NO: 144 from WO2019084064, respectively.

The heavy chain variable region (VH) amino acid sequence of AB85 is:

(SEQ ID NO: 37)
EVQLVQSGAEVKKPGESLKISCKGSGYSFTNYWIGWVRQMPGKGLEWMA
IINPRDSDTRYRPSFQGQVTISADKSISTAYLQWSSLKASDTAMYYCAR
HGRGYEGYEGAFDIWGQGTLVTVSS.

The VH CDR amino acid sequences of AB85 are as follows:

(VH CDR1; SEQ ID NO: 38)
NYWIG;
(VH CDR2; SEQ ID NO: 39)
IINPRDSDTRYRPSFQG;
and
(VH CDR3; SEQ ID NO: 40)
HGRGYEGYEGAFDI.

The light chain variable region (VL) amino acid sequence of AB85 is:

(SEQ ID NO: 41)
DIQMTQSPSSLSASVGDRVTITCRSSQGIRSDLGWYQQKPGKAPKLLIY
DASNLETGVPSRFSGSGSGTDFTLTISSLQPEDFATYYCQQANGFPLTF
GGGTKVEIK.

The VL CDR amino acid sequences of AB85 are as follows:

(VL CDR1; SEQ ID NO: 42)
RSSQGIRSDLG;
(VL CDR2; SEQ ID NO: 43)
DASNLET;
and
(VL CDR3; SEQ ID NO: 44)
QQANGFPLT .

FSI-174 is an anti-CD117 antibody being developed in combination with 5F9 as a non-toxic transplant conditioning regimen, as well as a treatment for targeted hematologic malignancies. The sequences of FSI-174 are disclosed in PCT Application Publication No. 2020/112687, U.S. Patent Application Publication No. 20200165337, and U.S. Pat. No. 11,041,022. In particular embodiments, an anti-CD117 antibody comprises the three CDRs or variable heavy chain regions present in any of AH1, AH2, AH3, AH4, or AH5 disclosed therein, and/or the three CDRs or variable heavy chain regions present in any of AL1 or AL2 disclosed therein.

In certain embodiments, the CDRs present in FSI-174 and related antibodies are as follows:

VH CDR1 =
(SEQ ID NO: 45)
SYNMH;
VH CDR2 =
(SEQ ID NO: 46)
VIYSGNGDTSY(A/N)QKF(K/Q)G;
VH CDR3 =
(SEQ ID NO: 26)
ERDTREGN;
VL CDR1 =
(SEQ ID NO: 47)
RAS(D/E)SVDIYG(N/Q)SFMH;
VL CDR2 =
(SEQ ID NO: 28)
LASNLES;
and
VL CDR3 =
(SEQ ID NO: 29)
QQNNEDPYT.

A/N and the like indicate that the amino acid position may be either of the two amino acids, in this example, A or N. In certain embodiments, CDRs present in the heavy variable region are CDRs H1, H2 and H3 as derinea by Kapat:

H1 =
(SEQ ID NO: 45)
SYNMH;
H2 =
(SEQ ID NO: 48)
VIYSGNGDTSYAQKFKG;
H3 =
(SEQ ID NO: 26)
ERDTREGN;

and the CDRs present in the light variable region are CDRs L1, L2 and L3 as defined by Kabat:

L1 =
(SEQ ID NO: 49)
RASESVDIYGQSFMH;
L2 =
(SEQ ID NO: 28)
LASNLES;
and
L3 =
(SEQ ID NO: 29)
QQNNEDPYT,

respectively except that 1, 2, or 3 CDR residue substitutions is/are present selected from N to A at heavy chain position 60, K to Q at heavy chain position 64 and N to Q at light chain position 30, positions being numbered according to Kabat. In certain embodiments, the antibody comprises any of the heavy chain variable region sequences (AH2, AH3, AH4) and/or light chain variable chain region sequences provided below (AL2), or the CDRs therein shown underlined:

AH2:
(SEQ ID NO: 50)
QVQLVQSGAEVKKPGASVKVSCKASGYTFTSYMNHWVRQAPGQGLEWMG
VIYSGNGDTSYAQKFKGRVTITADKSTSTAYMELSSLRSEDTAVYYCAR
ERDTRFGNWGQGTLVTVSS
AH3:
(SEQ ID NO: 51)
QVQLVQSGAEVKKPGASVKVSCKASGYTFTSYMNHWVRQAPGQGLEWMG
VIYSGNGDTSYNQKFQGRVTITADKSTSTAYMELSSLRSEDTAVYYCAR
ERDTREGNWGQGTLVTVSS
AH4
(SEQ ID NO: 52)
QVQLVQSGAEVKKPGASVKVSCKASGYTFTSYMNHWVRQAPGQGLEWMG
VIYSGNGDTSYAQKFQGRVTITADKSTSTAYMELSSLRSEDTAVYYCAR
ERDTRFGNWGQGTLVTVSS
AL2:
(SEQ ID NO: 53)
DIVMTQSPLSLPVTPGEPASISCRASESVDIYGQSFMHWYQQKPGQPPK
LLIYLASNLESGVPDRFSGSGSGTDFTLKISRVEAEDVGVYYCQQNNED
PYTFGGGTKVEIK

In certain embodiments, the anti-CD117 antibody comprises the full heavy chain and/or full light chain of any of the antibodies disclosed herein, or an amino acid sequence having at least 85%, at least 90%, at least 95%, at least 98%, at least 99% identity to a heavy or light chain disclosed herein, e.g., a JSP191 heavy or light chain. In certain embodiments, the anti-CD117 antibody comprises the variable region of a heavy chain and/or light chain of any of the antibodies disclosed herein, or an amino acid sequence having at least 85%, at least 90%, at least 95%, at least 98%, at least 99% identity to the variable region of a heavy or light chain disclosed herein, e.g., a JSP191 heavy or light chain variable region. In certain embodiments, the anti-CD117 antibody comprises a heavy chain and/or a light chain comprising one or more CDRs of an antibody disclosed herein, e.g., two, three, four, five or six CDRs of an antibody disclosed herein, e.g., a JSP191 antibody. In particular embodiments, the anti-CD117 antibody comprises a heavy chain or variable region thereof comprising one, two, or three heavy chain CDRs disclosed herein, e.g., a JSP191 heavy chain. In particular embodiments, the anti-CD117 antibody comprises a light chain or variable region thereof comprising one, two, or three light chain CDRs disclosed herein, e.g., a JSP191 light chain.

In particular embodiments, the antibody binds to a region of wild-type CD117 or an epitope of wild-type CD117 that is modified in a modified CD117 polypeptides disclosed herein. In particular embodiments, the antibody does not bind a modified CD117 polypeptide disclosed herein, or binds to a modified CD117 polypeptide disclosed herein with reduced affinity, e.g, less than 50%, less than 25%, or less than 10%. Antibody affinity to a particular polypeptide, such as wild-type CD117 or a modified CD117 may be determined, e.g., by measuring the equilibrium dissociation constant between the antibody and its antigen (K_{D}), which may be determined by routine methods in the art, e.g., by surface plasmon resonance, as described in Hearty, Stephen, Paul Leonard, and Richard O'Kennedy “Measuring antibody—antigen binding kinetics using surface plasmon resonance” Antibody Engineering: Methods and Protocols, Second Edition (2012): 411-442.

In particular embodiments, the modified cell expressing the modified CD117 polypeptide is capable of proliferating or surviving in the presence of an anti-CD117 antibody, e.g., an anti-CD117 antibody that blocks or inhibits binding of SCF to CD117 on the cell surface. In certain embodiments, the anti-CD117 antibody is capable of inhibiting proliferation of or inducing death or apoptosis of a cell expressing only the wild-type CD117 and not a modified CD117 polypeptide disclosed herein. In particular embodiments, the anti-CD117 antibody is selected from the group consisting of: SR1, 2B8, ACK2, YB5-B8, 57A5, 104D2, JSP191, CDX-0159, MGTA-117 (AB85), and FSI-174. In particular embodiments, the antibody is JSP191. Thus, in particular embodiments, the modified CD117 polypeptides disclosed herein, when expressed on the surface of an HSC and/or HSPC, are capable of substantially binding SCF in the presence of an anti-CD117 antibody, e.g., an anti-CD117 antibody that inhibits binding of SCF to endogenous, wild-type CD117 on the cell surface. Similarly, in particular embodiments, the modified CD117 polypeptides disclosed herein, when expressed on a HSC and/or HSPC surface, are capable of intracellular signaling when bound by SCF, in the absence of and in the presence of an anti-CD117 antibody e.g., an anti-CD117 antibody that inhibits binding of SCF to endogenous, wild-type CD117 on the cell surface. In particular embodiments, SCF binding and/or SCF-mediating signaling in the modified cell comprising the modified CD117 polypeptide is not substantially reduced in the presence of the anti-CD117 antibody, e.g., binding and/or signaling of the modified cell expressing the modified CD117 polypeptide is at least 40%, at least 50%, at least 60%, at least 70%, at least 80%, or at least 90% of the level of binding and/or signaling observed in the same cell type that is not modified, e.g., only expresses wild-type CD117.

For engraftment purposes, a composition comprising HSCs and/or HSPCs is administered to a patient. Such methods are well known in the art. The stem cells are optionally, although not necessarily, purified. Abundant reports explore various methods for purification of stem cells and subsequent engraftment, including flow cytometry; an isolex system (Klein et al. (2001) Bone Marrow Transplant. 28(11):1023-9; Prince et al. (2002) Cytotherapy 4(2):137-45); immunomagnetic separation (Prince et al. (2002) Cytotherapy 4(2):147-55; Handgretinger et al. (2002) Bone Marrow Transplant. 29(9):731-6; Chou et al. (2005) Breast Cancer. 12(3):178-88); and the like. Each of these references is herein specifically incorporated by reference, particularly with respect to procedures, cell compositions and doses for hematopoietic stem cell transplantation.

The present disclosure also includes pharmaceutical compositions comprising one or more modified CD117 polypeptides, one or more polynucleotides or vectors comprising a sequence encoding a modified CD117 polypeptide (e.g., a modified mRNA), or a modified cell comprising a polynucleotide or vector encoding a modified CD117 polypeptide and/or expressing a modified CD117, in combination with one or more pharmaceutically acceptable diluent, carrier, or excipient.

The present invention discloses a pharmaceutical composition comprising a modified cell comprising a modified CD117 polypeptide (or nucleic acid sequence encoding the modified CD117 polypeptide) described herein and one or more pharmaceutically acceptable diluent, carrier, or excipient. In particular embodiments, the cell is a heterologous cell or an autologous cell obtained from the subject to be treated. In particular embodiments, the cell is a stem cell, e.g., a HSC and/or HSPC. In certain embodiments, the pharmaceutical composition further comprises one or more additional active agents. In certain embodiments, the one or more additional active agent comprises an anti-CD117 antibody. In particular embodiments, the anti-CD117 antibody is selected from the group consisting of: SR1, 2B8, ACK2, YB5-B8, 57A5, 104D2, JSP191, CDX-0159, MGTA-117 (AB85), and FSI-174. In particular embodiments, the antibody is JSP191. In certain embodiments, the one or more additional active agent comprises one or more anti-CD47, anti-CD40L, anti-CD122, anti-CD4, and/or anti-CD8 antibody.

The polynucleotides, polypeptides, and cells described herein can be combined with pharmaceutically-acceptable carriers, diluents and reagents useful in preparing a formulation that is generally safe, non-toxic, and desirable, and includes excipients that are acceptable for mammalian, e.g., human or primate, use. In certain embodiments, the pharmaceutical composition is a solution or suspension comprising modified cells disclosed herein. Examples of such carriers or diluents include, but are not limited to, water, saline, Ringer's solutions, dextrose solution, and 5% human serum albumin. Supplementary active compounds can also be incorporated into the formulations. Solutions or suspensions used for the formulations can include a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates; detergents such as Tween 20 to prevent aggregation; and compounds for the adjustment of tonicity such as sodium chloride or dextrose. The pH can be adjusted with acids or bases, such as hydrochloric acid or sodium hydroxide. In particular embodiments, the pharmaceutical compositions are sterile. For intravenous administration, suitable carriers include physiological saline, bacteriostatic water, or phosphate buffered saline (PBS). In certain embodiments, it is stable under the conditions of manufacture and storage and is preserved against the contaminating action of microorganisms such as bacteria and fungi. The carrier can be, e.g., a solvent or dispersion medium containing, for example, water, ethanol, polyol (for example, glycerol, propylene glycol, and liquid polyethylene glycol, and the like), and suitable mixtures thereof. In some cases, the composition is sterile and may be fluid to the extent that easy syringability exists. Prevention of the action of microorganisms can be achieved by various antibacterial and antifungal agents, for example, parabens, chlorobutanol, phenol, ascorbic acid, thimerosal, and the like. In certain embodiments, a pharmaceutical composition include isotonic agents, for example, sugars, polyalcohols such as mannitol, sorbitol, sodium chloride in the composition. Prolonged absorption of the internal compositions can be brought about by including in the composition an agent which delays absorption, for example, aluminum monostearate and gelatin.

### Methods of Use

In further aspects, the disclosure provides methods of treating a mammalian subject in need thereof, comprising administering to the subject modified cells, e.g., HSCs and/or HSPCs, comprising a modified CD117 polypeptide described herein and/or a nucleic acid encoding the modified CD117 polypeptide. In particular embodiments, the subject is in need of HCT or a hematopoietic stem cell transplant. The transplant may be autologous, allogeneic, or xenogeneic, including without limitation allogeneic haploidentical stem cells, mismatched allogeneic stem cells, genetically engineered autologous or allogeneic cells, etc. In particular embodiments, the modified HSCs are infused into the subject, e.g., by intravenous infusion, e.g., through a central vein over a period of several minutes to several hours.

Where the donor is allogeneic to the recipient, the HLA type of the donor and recipient may be tested for a match, or haploidentical cells may be used. In certain embodiments, cells obtained from HLA-haploidentical donors or HLA-identical donors are used. HLA-haploidentical donors can be manipulated by CD34 or CD34/CD90 selection. For HLA matching, traditionally, the loci critical for matching are HLA-A, HLA-B, and HLA-DR. HLA-C and HLA-DQ are also now considered when determining the appropriateness of a donor. A completely matched sibling donor is generally considered the ideal donor. For unrelated donors, a complete match or a single mismatch is considered acceptable for most transplantation, although in certain circumstances, a greater mismatch is tolerated. Preferably matching is both serologic and molecular. Where the donor cells are from umbilical cord blood, the degree of tolerable HLA disparity is much greater, and a match of three or four out of the six HLA-A, HLA-B and HLA-DRBI antigens is typically sufficient for transplantation. Immunocompetent donor T cells may be removed using a variety of methods to reduce or eliminate the possibility that graft versus host disease (GVHD) will develop.

The HCT methods disclosed use modified HSCs comprising a modified CD117 polypeptide or nucleic acid encoding the modified CD117 polypeptide. The methods may result in reduced toxicity, reduced morbidity, or reduced graft-versus-host disease, as compared to HCT wherein a subject is administered HSCs that do not comprise the modified CD117 polypeptide or nucleic acid encoding the modified CD117 polypeptide. The methods of the invention are also believed to provide for improved engraftment of stem cells after transplantation into a recipient.

In particular embodiments of any of the methods of treatment disclosed herein, the subject is administered a conditioning regimen to facilitate or increase engraftment of the modified cells. In certain embodiments, the conditioning regimen depletes endogenous normal or disease HSCs of the subject. Conditioning regimens may be given prior to transplant to reduce the number of blood stem cells in the bone marrow to make space for donor blood stem cells to engraft and cure the patient. Typically, the conditioning regimen is administered prior to and/or concurrent with the administering of the pharmaceutical composition. In certain embodiments, the conditioning regimen comprises administration of an anti-CD117 antibody, wherein the anti-CD117 antibody depletes endogenous HSCs expressing wild-type CD117, but the anti-CD117 antibody does not deplete the administered modified HSCs. In particular embodiments, the anti-CD117 antibody is selected from the group consisting of: SR1, 2B8, ACK2, YB5-B8, 57A5, 104D2, JSP191, CDX-0159, MGTA-117 (AB85), and FSI-174. In particular embodiments, the antibody is JSP191. In particular embodiments, the conditioning regimen comprises an anti-CD117 antibody alone. In particular embodiments, the subject is administered the anti-CD117 antibody prior to administration of the modified HSCs, e.g., as a single dose.

An effective dose of anti-CD117 antibody is the dose that depletes endogenous hematopoietic stem cells. The effective dose will depend on the individual and the specific antibody, but will generally be up to about 100 μg/kg body weight, up to about 250 μg/kg, up to about 500 μg/kg, up to about 750 μg/kg, up to about 1 mg/kg, up to about 1.2 mg/kg, up to about 1.5 mg/kg, up to about 3 mg/kg, up to about 5 mg/kg, up to about 10 mg/kg. In some embodiments, the subject is administered about 0.01 mg/kg to about 2 mg/kg of the anti-CD117 antibody, e.g., JSP191, and optionally the subject is administered about 0.1 mg/kg to about 1 mg/kg of the anti-CD117 antibody, e.g., JSP191. In some embodiments, anti-CD117 antibody may be administered to a subject in a dose about 0.01 mg/kg to about 2 mg/kg of the subject's body weight, or about 0.1 mg/kg to about 1 mg/kg of the subject's body weight. In some embodiments, the anti-CD117 signaling antibodies are administered in a dose of about 0.6 mg/kg.

In certain embodiments, the conditioning regimen comprises administration of an anti-CD117 antibody in combination with one or more additional antibodies. In certain embodiments, the one or more additional antibodies comprise one or more of: anti-CD47, anti-CD40L, anti-CD122, anti-CD4, and/or anti-CD8 antibody.

In certain embodiments, the conditioning regimen comprises administration of an anti-CD117 antibody, alone or in combination with a myeloablative (MA) conditioning, reduced intensity conditioning (RIC), or other non-MA (NMA) conditioning regimen. In certain embodiments, the conditioning regimen is a genotoxic conditioning regimen and/or may comprise one or more of: chemotherapy (optionally a nucleoside analog and/or an alkylating agent), monoclonal antibody therapy, and radiation, optionally radiation to the entire body.

However, in other embodiments, the subject is not administered a myeloablative or genotoxic conditioning regimen prior to or concurrent with the administering of the pharmaceutical composition. For example, the recipient may be immunocompetent, and the transplantation may be performed in the absence of myeloablative conditioning, i.e., in the absence of radiation and/or chemotherapeutic drugs. The recipient may be conditioned with the combined administration a set of agents selected according to the cells and HLA match.

The dose of stem cells, e.g., modified HSCs comprising a modified CD117 polypeptide and/or nucleic acid encoding a modified CD117 polypeptide, administered to a subject may depend on the purity of the infused cell composition, and the source of the cells. In particular embodiments, the dose administered is at least or about 1−2×10⁶ CD34+ cells/kg body weight for autologous and allogeneic transplants. Higher doses can include, for example, at least or about 3×10⁶, at least or about 4×10⁶, at least or about 5×10⁶, at least or about 6×10⁶, at least or about 7×10⁶, at least or about 8×10⁶, at least or about 9×10⁶, at least or about 10⁷ or more CD34+ cells/kg body weight for autologous and allogeneic transplants. Frequently, the dose is limited by the number of available cells, and the methods disclosed encompass delivering less cells when necessary or limited. Typically, regardless of the source, the dose is calculated by the number of CD34+ cells present. The percent number of CD34+ cells can be low for unfractionated bone marrow or mobilized peripheral blood; in which case the total number of cells administered may be higher.

In certain embodiments, a maximum number of CD3+ cells delivered with the modified HSC composition is not more than about 10⁷ CD3+ cells/kg of recipient body weight, not more than about 10⁶ CD3+ cells/kg of recipient body weight, not more than about 10⁵ CD3+ cells/kg of recipient body weight, or not more than about 10⁴ CD3+ cells/kg of recipient body weight. Alternatively, cell populations may be selected for expression of CD34 and CD90, which cell populations may be highly purified, e.g., at least about 85% CD34+ CD90+ cells, at least about 90% CD34+ CD90+ cells, at least about 95% CD34+ CD90+ cells and may be up to about 99% CD34+ CD90+ cells or more.

In certain embodiments, the disclosure includes a method of treating a mammalian subject in need thereof, comprising administering to the subject modified cells, e.g., HSCs and/or HSPCs, comprising a modified CD117 polypeptide disclosed herein. In particular embodiments, the subject is also administered a conditioning regimen to facilitate or increase engraftment of the modified cells following transplantation, wherein the conditioning regimen is administered prior to and/or or concurrent with and/or following the administering of the pharmaceutical composition. In particular embodiments, the conditioning regimen comprises administration of an anti-CD117antibody, e.g., any disclosed herein, to the subject. In some embodiments, the anti-CD117 antibody is administered to the subject prior to administration of the pharmaceutical composition to the subject. In particular embodiments, there is a “wash-out” period following administration of the anti-CD117 antibody and before administration of the modified cells (i.e., the HCT). This period of time allows clearance of the anti-CD117 antibody (or other agent used for conditioning). The period of time required for clearance of the ablative agent may be empirically determined, or may be based on prior experience of the pharmacokinetics of the agent. Historically, the time for clearance was usually the time sufficient for the level of ablative agent, e.g., anti-CD117 antibody, to decrease at least about 10-fold from peak levels, usually at least about 100-fold, 1000-fold, 10,000-fold, or more. However, since the modified cells being administered to the subject according to the methods disclosed herein comprise a modified CD117 polypeptide that is not bound by the ablative anti-CD117 antibody used for conditioning, the disclosed methods do not require a wash-out period, or require only a reduced wash-out period as compared to when unmodified cells are transplanted. In certain embodiments, the wash-out period is less than five days, less than four days, less than 3 days, less than two days, or less than one day. In certain embodiments, the method comprises administering the anti-CD117 antibody and the pharmaceutical composition or modified cells, e.g., modified HSCs and/or HSPCs, during an overlapping period of time or at about the same time. In particular embodiments, the method comprises also, or alternatively, administering the anti-CD117 antibody to the subject after administration of the pharmaceutical composition or modified cells, e.g., modified HSCs and/or HSPCs, optionally for a period of time of at least one day, at least two days, at least three days, at least four days, at least five days, or at least one week. This may continue to ablate endogenous HSCs and/or HSPCs following administration of the modified HSCs and/or HSPCs, thus allowing greater engraftment.

In one embodiment, the method comprises:
(i) selectively ablating endogenous hematopoietic stem cells in the subject by administering to the subject an anti-CD117 antibody, e.g., JSP-191;
(ii) optionally, waiting for a period of time following administration of the anti-CD117 antibody; and
(iii) following (ii), administering to the subject the pharmaceutical composition comprising the modified cells, e.g., modified HSCs and/or modified HSPCs, in a dose effective to achieve multilineage peripheral blood chimerism,
   In particular embodiments, the period of time is less than five days, less than four days, less than 3 days, less than two days, or less than one day, or there is no period of time.

In certain embodiments, the method of treating a subject in need of HCT comprises:
(i) administering a conditioning regimen to the subject, wherein the conditioning regimen comprises an anti-CD117 monoclonal antibody, e.g., JSP191; and
(ii) administering modified HSCs to the subject, wherein the modified HSCs comprise a modified CD117 polypeptide disclosed herein, wherein the modified CD117 polypeptide is expressed on the cell surface, and wherein the modified HSCs are not depleted by the conditioning regimen to the same extent as endogenous HSCs that comprise only wild type CD117 polypeptide.

In particular embodiments of the methods disclosed, the anti-CD117 antibody in any one of JSP191, AB85, CDX-0159, or FSI-174, or any other anti-CD117 antibody disclosed herein. In particular embodiments, the anti-CD117 antibody is JSP-191, and the modified CD117 comprises an amino acid modification, e.g., a substitution, of one or more of: E73, D121, R122, S123, Y125, and K203, or is within an epitope comprising any of these amino acids. In some embodiments, the anti-CD117 antibody is AB85, and the modified CD117 comprises an amino acid modification, e.g., a substitution, of one or more of Y259, S261, W262, Y269, and R271, or is within an epitope comprising any of the amino acids. In some embodiments, the anti-CD117 antibody is CDX-0159, and the modified CD117 comprises one or more amino acid modifications within one or more epitope on CD117 bound by CDX-0159. In some embodiments, the anti-CD117 antibody is FSI-174, and the modified CD117 comprises one or more amino acid modifications within one or more epitope on CD117 bound by FSI-174. In particular embodiments, the anti-CD117 antibody is FSI-174, and the modified CD117 comprises an amino acid modification, e.g., a substitution, of one or more of: E73, D121, R122, S123, Y125, and K203, or is within an epitope comprising any of these amino acids. In certain embodiments, the modification is an amino acid substitution, and in some embodiments, the amino acid substitution is an alanine substitution, a conservative substitution, or a non-conservative substitution. In particular embodiments, the one or more amino acid modifications comprise one or more amino acid substitutions or deletions of an amino acid residue selected from the following in human CD117: E73, D121, R122, S123, Y125, K203, Y259, S261, W262, Y269, or R271. In certain embodiments, the one or more amino acid modifications comprise one or more amino acid substitutions, e.g., of any of these residues.

In some embodiments, the transplantation is performed in the absence of myeloablative conditioning. In some embodiments the recipient is immunocompetent. The administration of the pre-transplantation conditioning regimen is repeated as necessary to achieve the desired level of ablation. Following transplantation with donor stem cells, the recipient may be a chimera or mixed chimera for the donor cells.

The methods disclosed herein may be used to treat a variety of indications amenable to stem cell transplantation. For example, in certain embodiments, the methods comprise: (i) conditioning a subject by administration of an anti-CD117 antibody, e.g., JSP191, alone or in combination with one or more additional conditioning agent; and administering to the subject modified HSPCs/HSCs comprising a modified CD117 disclosed herein, e.g., CD117 E73A. In particular embodiments, the modified CD117 cells comprise one or more additional modifications. For example, they may comprise one or more introduced genes to replace a missing, mutated, or dysfunctional gene or protein product in a diseased cell.

In particular embodiments, HCT methods disclosed herein are used to treat a disease or disorder selected from the group consisting of: a cancer, a cardiac disorder, a neural disorder, an autoimmune disease, an immunodeficiency, a metabolic disorder, hemoglobinopathies, and a genetic disorder. In particular embodiments, they are used to treat any of the following disorders: multiple myeloma, non-Hodgkin lymphoma, Hodgkin disease, acute myeloid leukemia, neuroblastoma, germ cell tumors, and autoimmune disorders, e.g., systemic lupus erythematosus (SLE), systemic sclerosis, or amyloidosis, for example, by autologous HCT. In particular embodiments, they are used to treat any of the following disorders: acute myeloid leukemia, acute lymphoblastic leukemia, chronic myeloid leukemia; chronic lymphocytic leukemia, myeloproliferative disorders, myelodysplastic syndromes, multiple myeloma, non-Hodgkin lymphoma, Hodgkin disease, aplastic anemia, pure red cell aplasia, paroxysmal nocturnal hemoglobinuria, Fanconi anemia, thalassemias, thalassemia major, sickle cell anemia, combined immunodeficiency, severe combined immunodeficiency (SCID), Wiskott-Aldrich syndrome, hemophagocytic lymphohistiocytosis (HLH), inborn errors of metabolism (e.g., mucopolysaccharidosis, Gaucher disease, metachromatic leukodystrophies, and adrenoleukodystrophies), epidermolysis bullosa, severe congenital neutropenia, Shwachman-Diamond syndrome, Diamond-Blackfan anemia, leukocyte adhesion deficiency, and the like, for example, by allogeneic HCT.

In particular embodiments, the methods disclosed are used to treat a solid tissue cancer or a blood cancer, such as a leukemia, a lymphoma, or a myelodysplastic syndrome. In particular embodiments, the leukemia is acute myeloid leukemia (AML). In particular embodiments, the lymphoma is diffuse large B-cell lymphoma.

In particular embodiments, the methods disclosed are used to treat an immunodeficiency. In particular embodiments, the immunodeficiency is severe combined immunodeficiency (SCID). In particular embodiments, the immunodeficiency is immunoglobulin G subclass deficiency, selective immunoglobulin A deficiency, DiGeorge syndrome, hyper-immunoglobulin M (HIGM) syndrome, selective IgM deficiency, Wiskott-Aldrich syndrome, or X-linked agammaglobulinemia (XLA).

In particular embodiments, the methods disclosed are used to treat a genetic disorder. In particular embodiments, the genetic disorder is sickle cell disease or Fanconi anemia. Sickle cell diseases that may be treat include, but are not limited to: HbS disease; drepanocytic anemia; meniscocytosis, and chronic hemolytic anemia.

In certain embodiments of any of the HCT methods disclosed, the method further comprises administering to the subject a therapeutic agent for treatment of the disease or disorder being treated by the HCT method.

### EXAMPLES

### Example 1

### Identification of Anti-CD117 Antibody Epitopes on CD117

Epitopes on CD117 bound by various anti-CD117 antibodies were identified by alanine scanning mutagenesis of the wild type human CD117 protein.

HEK-293T cells were transfected with a wild type (WT) construct of the CD117 protein or with vector alone in 384-well format, followed by confirmation of cellular expression via high-throughput flow cytometry. The MAbs tested included JSP191 and AB85; the ligand tested included AF488-conjugated stem cell factor (SCF), and the control MABs tested included YB5.88 (Invitrogen, Cat. No. 14-1179-82) and 104D2 (BioLegend, Cat. No. 313202), all of which bind WT CD117.

Shotgun Mutagenesis epitope mapping services were provided by Integral Molecular (Philadelphia, PA) as described in Davidson and Doranz, 2014. Briefly, a mutation library of the target protein was created by high-throughput, site-directed mutagenesis. Each residue was individually mutated to alanine, with alanine codons mutated to serine. The mutant library was arrayed in 384-well microplates and transiently transfected into HEK293T cells. Following transfection, cells were incubated with the indicated antibodies at concentrations pre-determined using an independent immunofluorescence titration curve on wild type protein. MAbs were detected using an Alexa Fluor 488-conjugated secondary antibody and mean cellular fluorescence was determined using Intellicyt iQue flow cytometry platform. Mutated residues were identified as being critical to the MAb epitope if they did not support the reactivity of the test MAb but did support the reactivity of the reference MAb. This counterscreen strategy facilitates the exclusion of mutants that are locally misfolded or that have an expression defect.

Serial dilution of each monoclonal antibody (Mab) or ligand were tested for immunoreactivity against cells expressing target protein (WT) or vector alone to determine optimal screening conditions for each Mab or ligand based on raw signal values and signal-to-background calculations. AlexaFluor 488®-labeled goat anti-human IgG antibody was used as secondary detection antibody for JSP191 and AB85, and AlexaFluor 488®-labeled goat anti-mouse IgG antibody was used as secondary detection antibody for YB5.B8 and 104D2.

Library screens of very-high-affinity MAbs sometimes fail to yield critical residues for antibody binding. Conversion of a high-affinity MAb to a Fab usually weakens binding sufficiently to allow identification of critical residues for binding. Serial dilution of Fabs of MAbs were also tested for immunoreactivity against cells expressing target protein (WT) or vector alone to determine optimal screening conditions for each Fab based on raw signal values and signal-to-background calculations. The Fabs tested included two JSP191 Fab (JSP191Fab and JSP191Fab2) and an AB85 Fab. AlexaFluor 488®-labeled goat anti-human IgG F(ab′)2 antibody was used as secondary detection antibody for JSP191 and AB85 Fabs.

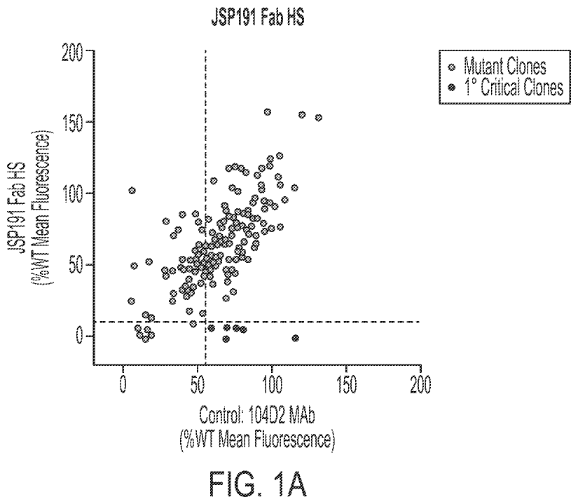

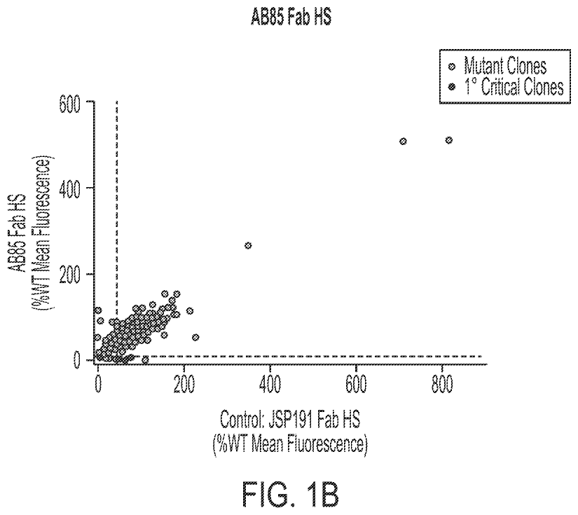

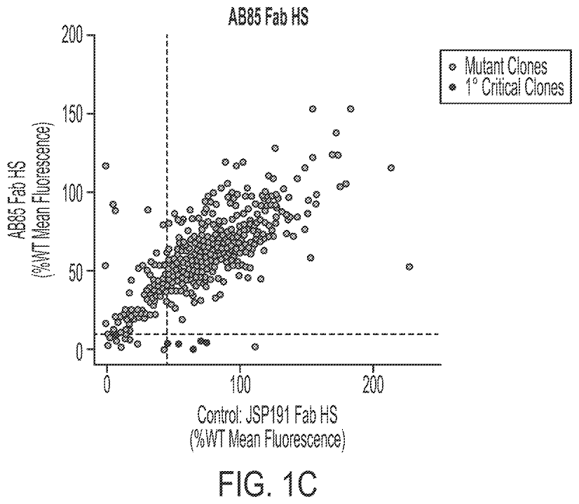

Binding of each test Ab to each mutant clone in the alanine scanning library was determined, in duplicate, by high-throughput flow cytometry. For each point, background fluorescence was subtracted from the raw data, which were then normalized to Ab reactivity with WT target protein. For each mutant clone, the mean binding value was plotted as a function of expression (represented by control reactivity) (FIGS. 1A-C). To identify preliminary primary critical clones (lower right quadrant of graphs), a threshold (dashed lines) of >55% WT binding to control Ab and <10% WT binding to test Abs was applied for JSP191, and >45% WT binding to control Ab and <10% WT binding to test Abs for AB85.

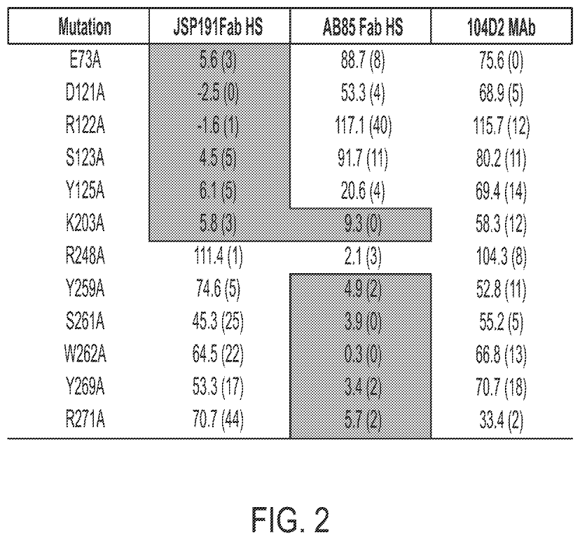

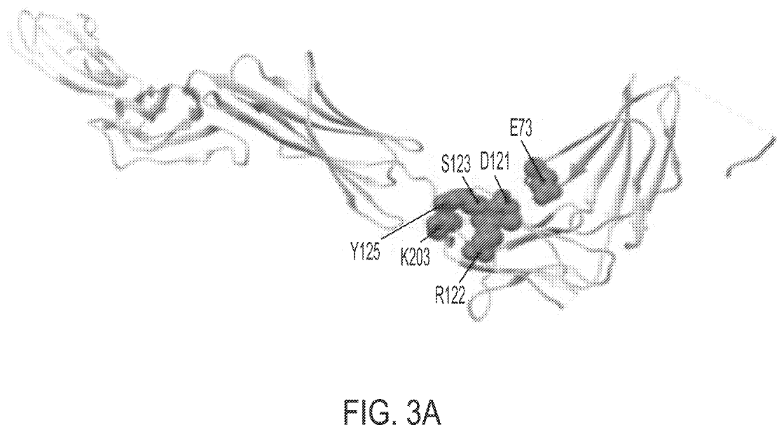

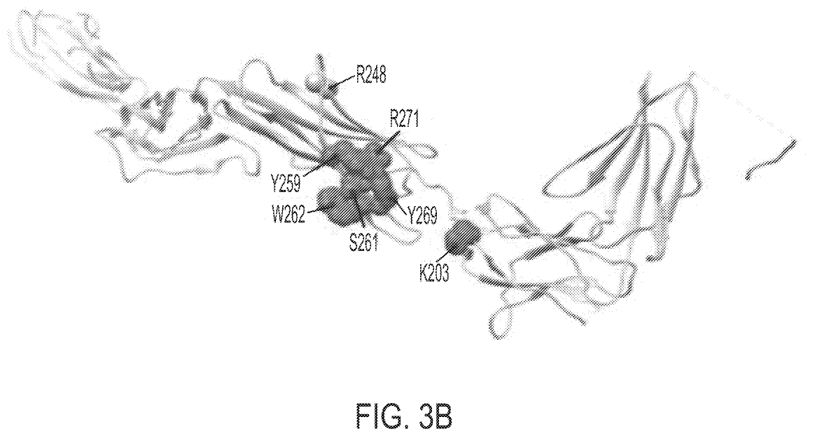

Mean binding reactivities (and ranges) are listed for all identified critical residues in FIG. 2. Critical residues for Ab binding (shaded) were residues whose mutations were negative for binding to test Abs, but positive for binding to control antibody. Although mutant clone R248A showed a loss of binding for AB85, we do not consider it critical, as R248 appears to be an internal residue that is distant from the other epitope residues (see FIGS. 3A-B).

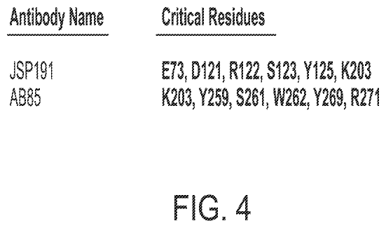

Critical residues whose mutation gave the lowest reactivities with specific antibodies are highlighted in bold and underlined in FIG. 4. Validated critical residues represent amino acids whose side chains make the highest energetic contributions to the antibody-epitope interaction (Bogan and Thorn, 1998; Lo Conte et al., 1999); therefore, the highlighted residues are likely the major energetic contributors to binding.

### Example 2

Effects of CD117 mRNA Expression

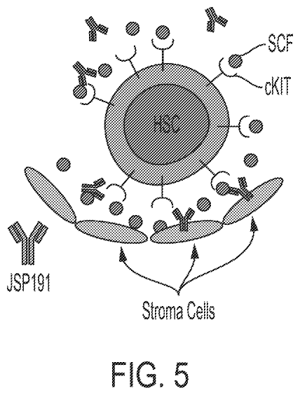

CD117 (cKit) binds stem cell factor (SCF) to regulate HSC survival, self-renewal, and differentiation, as diagrammed in FIG. 5. Experiments were performed to assess wild type and modified CD117 mRNAs, expressed from DNA templates provided in FIG. 6 and SEQ ID NOS: 5-7. In brief, human CD34+ and/or Ba/F3 cell lines were transfected with various mRNA constructs encoding wild type CD117 (produced from templates comprising SEQ ID NOs: 5 and 6), CD117 E73A from (produced from a template comprising SEQ ID NO: 7), or another CD117 with an amino acid substitution not identified as critical for JSP191 binding, (KG2-DV and KG2-DV-5moU). The sequences of SEQ ID NOS: 5-7 are the DNA templates and include a T7promoter, CleanCapAG, Kozak sequence, an HBA1 5′ UTR, a TAATGA double stop codon, an HBB1 3′ UTR. SEQ ID NOs: 5-7 do not include the poly-adenosine tail of 70 nucleotides that is also present in the template and resulting mRNAs. The mRNAs correspond to mRNA expressed from these templates, so their sequences comprise Us instead of Ts. Controls included no transfection or null transfection where indicated.

cKIT Expression

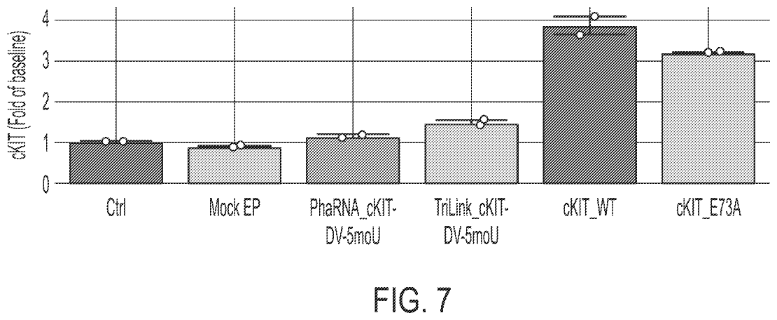

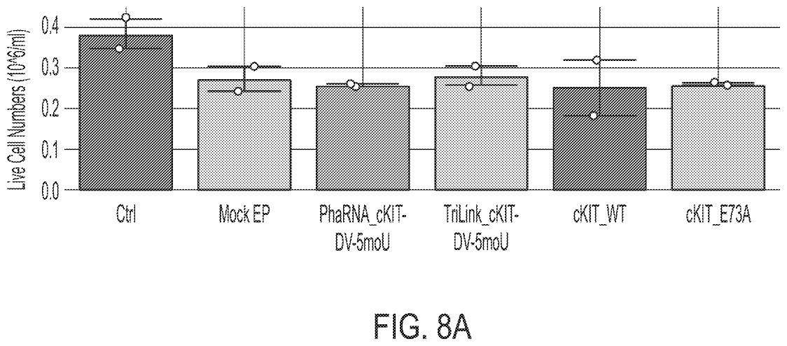

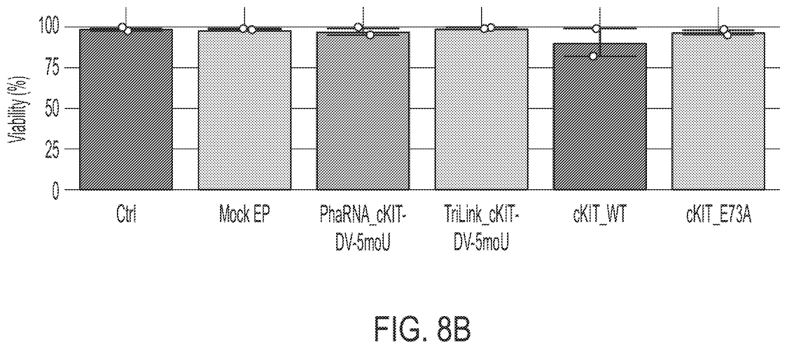

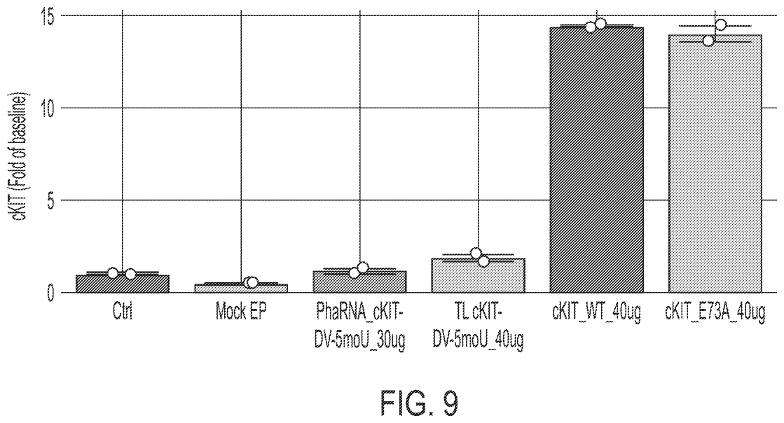

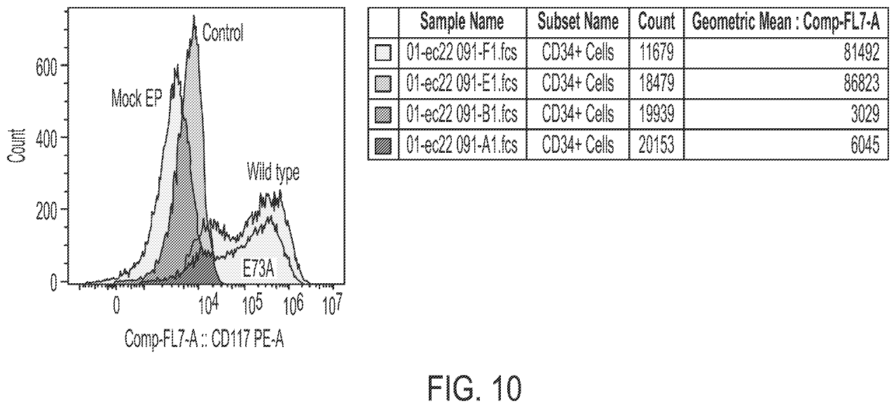

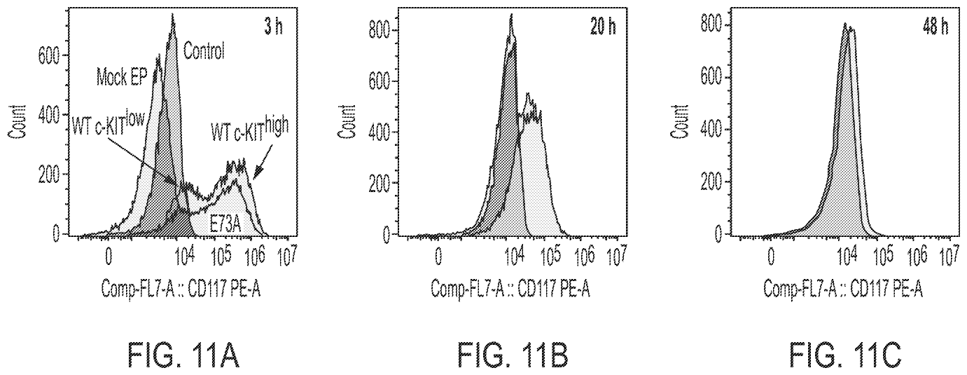

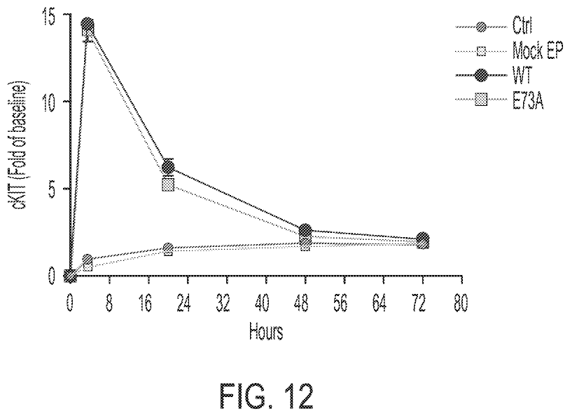

The codon optimized mRNAs provided herein, e.g., cKIT_WT_co1 (produced from SEQ ID NO: 6) and cKIT_E73A_co1 (produced from SEQ ID NO: 7), demonstrated higher expression levels than the PhaRNA (KG2-DV) and TriLINK (KG2-DV-5moU) cKIT mRNAs, as determined by CD117 (cKIT) levels measured 20 hours after electroporation (FIG. 7) in human CD34+ cells. When measured just 3 hours after electroporation, the mRNAs provided herein demonstrated significant improvement in in CD117 expression levels over the aforementioned mRNAs (FIG. 9). At the same time, the mRNAs provided herein showed similar levels of CD34+ cell viability to the comparison mRNAs (KG2-DV and KG2-DV-5moU) 20 hours after electroporation (FIGS. 8A and 8B). FIG. 10 is a graph of cell count versus CD117 expression in human CD34+ cells expressing mock electroporated (mock EP), null (control), wild type CD117, and CD117 E73A mRNAs. FIGS. 11A-11C and FIG. 12 demonstrate that CD34+ cells expressing wild type CD117and E73A mRNAs exhibited two distinct levels of transient CD117 expression at 3 hours after electroporation, which resolved by 20 hours after electroporation.

### Resistance to JSP 191

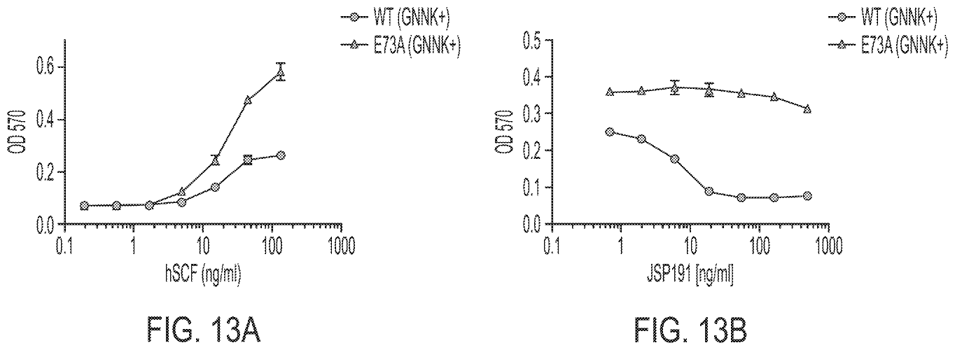

To determine the effects of the E73A mutant CD117 expression on JSP191 resistance in cells, E73A CD117 mutant lentivirus constructs were transfected into Ba/F3 cells. The Ba/F3 cells grew in response to human stem cell factor, and independently of IL3 stimulation (FIG. 13A). Notably, the E73A mutant expressing cells were more growth responsive to stem cell factor than were the wild type (FIG. 13A). Expression of E73A led to nearly complete resistance to JSP191 (FIG. 13B), even at higher concentrations.

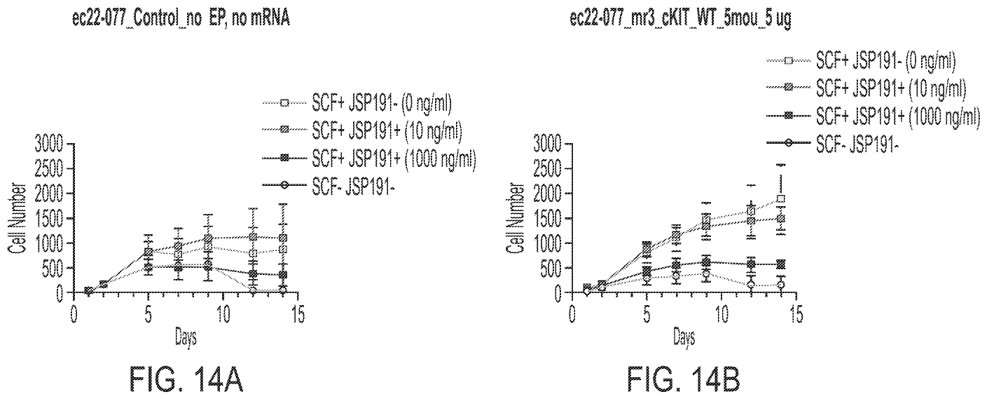

FIGS. 14A-14B show the effects of CD117 expression on human CD34+ cell growth in the presence of stem cell factor (SCF) and the JSP91 antibody. FIG. 14A shows the growth of cells transfected without mRNA. FIG. 14B shows the growth of cells transfected with non-codon optimized wild type CD117 mRNA (as encoded by SEQ ID NO: 54).

### REFERENCES

Bogan, A.A. and Thorn, K.S. (1998). Anatomy of hot spots in protein interfaces. J. Mol. Biol. 280, 1-9.
Davidson, E. and Doranz, B.J. (2014). A high-throughput shotgun mutagenesis approach to mapping B-cell antibody epitope. Immunology 143, 13-20.
Lo Conte, L., Chothia, C., and Janin, J. (1999). The atomic structure of protein-protein recognition sites. J. Mol. Biol. 285, 2177-2198.
Yuzawa, S., Opatowsky, Y., Zhang, Z., Mandiyan, V., Lax, I., Schlessinger, J. (2007). Structural basis for activation of the receptor tyrosine kinase KIT by Stem Cell Factor. Cell 130:323-334.

The various embodiments described above can be combined to provide further embodiments.

Aspects of the embodiments can be modified, if necessary to employ concepts of the various patents, patent applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure

All of the U.S. patents, U.S. patent application publications, U.S. patent applications, foreign patents, foreign patent applications, and non-patent publications referred to in this specification and/or listed in the Application Data Sheet are incorporated herein by reference, in their entirety.

1. A modified CD117 polypeptide comprising one or more amino acid modifications as compared to a wild type CD117 polypeptide, optionally wherein the one or more amino acid modifications inhibit or reduce binding of an anti-CD117 antibody to the modified CD117 polypeptide expressed in cells as compared to the binding of the antibody to wild type CD117 polypeptide expressed in cells and/or do not substantially inhibit or reduce binding of stem cell factor (SCF) to the modified CD117 polypeptide expressed in cells as compared to the binding of SCF to the wild type CD117 polypeptide expressed in cells.

2. The modified CD117 polypeptide of claim 1, wherein the one or more amino acid modifications inhibit or reduce binding of the anti-CD117 antibody to the modified CD117 polypeptide expressed in cells as compared to the binding of the antibody to the wild type CD117 polypeptide expressed in cells.

3. The modified CD117 polypeptide of claim 1 or claim 2, wherein the one or more amino acid modifications do not substantially inhibit or reduce binding of stem cell factor (SCF) to the modified CD117 polypeptide expressed in cells as compared to the binding of SCF to the wild type CD117 polypeptide expressed in cells.

4. The modified CD117 polypeptide of any one of claims 1-3, wherein the one or more amino acid modifications do not substantially inhibit or reduce CD117 signaling, optionally in response to SCF binding, by the modified CD117 polypeptide expressed in cells as compared to CD117 signaling by the wild type CD117 polypeptide expressed in cells.

5. The modified CD117 polypeptide of any one of claims 1-4, wherein the modified CD117 polypeptide substantially retains kinase activity in cells, optionally in response to SCF binding, as compared to the kinase activity of the wild type CD117 polypeptide in cells.

6. The modified CD117 polypeptide of any one of claims 1-5, wherein the anti-CD117 antibody comprises at least five, optionally six, CDRs present in any one of JSP191, AB85, CDX-0159, or FSI-174.

7. The modified CD117 polypeptide of claim 6, wherein the anti-CD117 antibody in any one of JSP191, AB85, CDX-0159, or FSI-174.

8. The modified CD117 polypeptide of claim 7, wherein the anti-CD117 antibody is JSP-191.

9. The modified CD117 polypeptide of any one of claims 1-8, wherein the one or more amino acid modifications comprise one or more amino acid substitutions, insertions, or deletions.

10. The modified CD117 polypeptide of claim 9, wherein one or more of the amino acid modifications are present within surface exposed amino acid residues of the extracellular domain of the wild type CD117 polypeptide.

11. The modified CD117 polypeptide of claim 9 or claim 10, wherein the one or more amino acid substitutions disrupt binding of the CD117 antibody to an epitope present in the wild type CD117 polypeptide bound by the anti-CD117 antibody.

12. The modified CD117 polypeptide of any one of claims 9-11, wherein the one or more amino acid modifications comprise one or more amino acid substitutions or deletions.

13. The modified CD117 polypeptide of claim 12, wherein the one of more amino acid substitutions or deletions comprises a substitution and/or deletion within an epitope of wild type human CD117 bound by an anti-CD117 antibody, optionally wherein the epitope comprises one or more of the following amino acids present in the wild type human CD117: E73, D121, R122, S123, Y125, K203, Y259, S261, W262, Y269, or R271.

14. The modified CD117 polypeptide of any one of claims 9-13, wherein the one of more amino acid substitutions or deletions comprises substitution or deletion of one or more of the following amino acids present in wild type human CD117: E73, D121, R122, S123, Y125, K203, Y259, S261, W262, Y269, or R271.

15. The modified CD117 polypeptide of any one of claims 1-14, wherein the modified CD117 polypeptide has at least 90%, at least 95%, at least 98%, or at least 99% sequence homology to the wild type CD117 polypeptide.

16. The modified CD117 polypeptide of any one of claims 1-15, wherein the wild type CD117 polypeptide is a wild type human CD117 polypeptide, optionally having one of the following amino acid sequences:
(SEQ ID NO: 1)
MRGARGAWDFLCVLLLLLRVQTGSSQPSVSPGEPSPPSIHPGKSDLIVRVGDEIRLLCTDP
GFVKWTFEILDETNENKQNEWITEKAEATNTGKYTCTNKHGLSNSIYVFVRDPAKLFLV
DRSLYGKEDNDTLVRCPLTDPEVTNYSLKGCQGKPLPKDLRFIPDPKAGIMIKSVKRAY
HRLCLHCSVDQEGKSVLSEKFILKVRPAFKAVPVVSVSKASYLLREGEEFTVTCTIKDVS
SSVYSTWKRENSQTKLQEKYNSWHHGDFNYERQATLTISSARVNDSGVFMCYANNTFG
SANVTTTLEVVDKGFINIFPMINTTVFVNDGENVDLIVEYEAFPKPEHQQWIYMNRTFTD
KWEDYPKSENESNIRYVSELHLTRLKGTEGGTYTFLVSNSDVNAAIAFNVYVNTKPEILT
YDRLVNGMLQCVAAGFPEPTIDWYFCPGTEQRCSASVLPVDVQTLNSSGPPFGKLVVQS
SIDSSAFKHNGTVECKAYNDVGKTSAYFNFAFKGNNKEQIHPHTLFTPLLIGFVIVAGM
MCIIVMILTYKYLQKPMYEVQWKVVEEINGNNYVYIDPTQLPYDHKWEFPRNRLSFGK
TLGAGAFGKVVEATAYGLIKSDAAMTVAVKMLKPSAHLTEREALMSELKVLSYLGNH
MNIVNLLGACTIGGPTLVITEYCCYGDLLNFLRRKRDSFICSKQEDHAEAALYKNLLHSK
ESSCSDSTNEYMDMKPGVSYVVPTKADKRRSVRIGSYIERDVTPAIMEDDELALDLEDL
LSFSYQVAKGMAFLASKNCIHRDLAARNILLTHGRITKICDFGLARDIKNDSNYVVKGN
ARLPVKWMAPESIFNCVYTFESDVWSYGIFLWELFSLGSSPYPGMPVDSKFYKMIKEGF
RMLSPEHAPAEMYDIMKTCWDADPLKRPTFKQIVQLIEKQISESTNHIYSNLANCSPNRQ
KPVVDHSVRINSVGSTASSSQPLLVHDDV;
or
(SEQ ID NO: 2)
MRGARGAWDFLCVLLLLLRVQTGSSQPSVSPGEPSPPSIHPGKSDLIVRVGDEIRLLCTDP
GFVKWTFEILDETNENKQNEWITEKAEATNTGKYTCTNKHGLSNSIYVFVRDPAKLFLV
DRSLYGKEDNDTLVRCPLTDPEVTNYSLKGCQGKPLPKDLRFIPDPKAGIMIKSVKRAY
HRLCLHCSVDQEGKSVLSEKFILKVRPAFKAVPVVSVSKASYLLREGEEFTVTCTIKDVS
SSVYSTWKRENSQTKLQEKYNSWHHGDFNYERQATLTISSARVNDSGVFMCYANNTFG
SANVTTTLEVVDKGFINIFPMINTTVFVNDGENVDLIVEYEAFPKPEHQQWIYMNRTFTD
KWEDYPKSENESNIRYVSELHLTRLKGTEGGTYTFLVSNSDVNAAIAFNVYVNTKPEILT
YDRLVNGMLQCVAAGFPEPTIDWYFCPGTEQRCSASVLPVDVQTLNSSGPPFGKLVVQS
SIDSSAFKHNGTVECKAYNDVGKTSAYFNFAFKEQIHPHTLFTPLLIGFVIVAGMMCIIV
MILTYKYLQKPMYEVQWKVVEEINGNNYVYIDPTQLPYDHKWEFPRNRLSFGKTLGAG
AFGKVVEATAYGLIKSDAAMTVAVKMLKPSAHLTEREALMSELKVLSYLGNHMNIVN
LLGACTIGGPTLVITEYCCYGDLLNFLRRKRDSFICSKQEDHAEAALYKNLLHSKESSCS
DSTNEYMDMKPGVSYVVPTKADKRRSVRIGSYIERDVTPAIMEDDELALDLEDLLSFSY
QVAKGMAFLASKNCIHRDLAARNILLTHGRITKICDFGLARDIKNDSNYVVKGNARLPV
KWMAPESIFNCVYTFESDVWSYGIFLWELFSLGSSPYPGMPVDSKFYKMIKEGFRMLSP
EHAPAEMYDIMKTCWDADPLKRPTFKQIVQLIEKQISESTNHIYSNLANCSPNRQKPVVD
HSVRINSVGSTASSSQPLLVHDDV.

17. A nucleic acid encoding the modified CD117 polypeptide of any one of claims 1-16.

18. The nucleic acid of claim 17, wherein the nucleic acid comprises RNA, DNA, or a combination thereof, optionally a modified mRNA.

19. The nucleic acid of claim 17 or claim 18, wherein the nucleic acid comprises:
a) a sequence of any one of SEQ ID NOS: 5-7, optionally further comprising a poly A tail, optionally an A70 poly A tail;
b) a sequence having at least 50%, at least 55%, at least 60%, at least 70%, at least 75%, at least 80%, at least 85%, at least 90%, at least 95%, at least 96%, at least 97%, at least 98%, or at least 99%, sequence identity to any one of SEQ ID NOS: 5-7, optionally further comprising a poly A tail, optionally an A70 poly A tail;
c) a coding sequence of any one of SEQ ID NOS: 5-7;
d) a sequence having at least 50%, at least 55%, at least 60%, at least 70%, at least 75%, at least 80%, at least 85%, at least 90%, at least 95%, at least 96%, at least 97%, at least 98%, or at least 99%, sequence identity to a coding sequence of any one of SEQ ID NOS: 5-7;
e) a sequence comprising the Cap sequence, Kozak sequence, and coding sequence of any one of SEQ ID NOS: 5-7, optionally further comprising a poly A tail, optionally an A70 poly A tail; and
f) a sequence having at least 50%, at least 55%, at least 60%, at least 70%, at least 75%, at least 80%, at least 85%, at least 90%, at least 95%, at least 96%, at least 97%, at least 98%, or at least 99%, sequence identity to a sequence comprising the Cap sequence, Kozak sequence, and coding sequence of any one of SEQ ID NOS: 5-7, optionally further comprising a poly A tail, optionally an A70 poly A tail.

20. The nucleic acid of any one of claims 17-19, wherein the nucleic acid is associated with one or more lipids, optionally wherein the nucleic acid is present within a lipid nucleic acid particle, a lipid nanoparticle, or a liposome.

21. A vector comprising the nucleic acid of any one of claims 17-20.

22. The vector of claim 21, wherein the vector is an expression vector.

23. The vector of claim 22, wherein the vector is a viral vector, optionally an AAV vector or a lentiviral vector.

24. The vector of any one of claims 21-23, wherein the vector is capable of transducing hematopoietic stem cells.

25. A modified cell comprising the modified CD117 polypeptide of any one of claims 1-16 and/or the nucleic acid of any one of claims 17-20.

26. The modified cell of claim 25, wherein the cell expresses both the modified CD117 polypeptide and a wild type CD117 polypeptide.

27. The modified cell of claim 25 wherein the cell was transduced with the vector of any one of claims 22-24.

28. The modified cell of claim 25, wherein the modified cell expresses the modified CD117 polypeptide but not a wild type CD117 polypeptide.

29. The modified cell of claim 25 or claim 28, wherein the endogenous gene encoding the wild type CD117 polypeptide was genetically modified to encode the modified CD117 polypeptide.

30. The modified cell of claim 29, wherein the modified cell was genetically modified by gene editing.

31. The modified cell of claim 30, wherein the gene editing was performed by a procedure selected from gene editing or base editing, optionally CRISPR-Cas9 gene editing, TALEN gene editing, zinc finger gene editing, or homing endonuclease or meganuclease gene editing.

32. The modified cell of any one of claims 25-31, wherein the cell is a stem cell or a pluripotent cell.

33. The modified cell of claim 19, wherein the stem cell is a hematopoietic stem cell (HSC) or a hematopoietic stem and progenitor cell (HSPC).

34. The modified cell of any one of claims 25-33, wherein the cell is CD34+, optionally wherein the cell is CD34+/CD90+, CD34+/CD38−, or CD34+/CD38−/CD90+, or CD34+CD133+.

35. The modified cell of any one of claims 25-34, wherein the cell is a human cell.

36. The modified cell of any one of claims 25-35, wherein the cell was obtained from a mammalian donor.

37. The modified cell of claim 36, wherein the mammalian donor is a subject is in need of a hematopoietic cell transplant (HCT).

38. The modified cell of claim 36, wherein the mammalian donor is a healthy donor.

39. The modified cell of any one of claims 36-38, wherein the cell obtained from the mammalian donor was modified ex vivo.

40. The modified cell of any one of claims 25-39, wherein the cell expresses the modified CD117 polypeptide, optionally wherein the modified cell expresses the modified CD117 polypeptide transiently or constitutively.

41. The modified cell of claim 40, wherein the modified CD117 polypeptide is expressed on the cell surface or in the cell membrane.

42. The modified cell of any one of claims 25-41, wherein the cell is capable of proliferating and/or survival in the presence of an anti-CD117 antibody.

43. The modified cell of claim 42, wherein the anti-CD117 antibody is capable of inhibiting proliferation and/or survival of a cell expressing only the wild-type CD117.

44. The modified cell of claim 42 or claim 43, wherein the anti-CD117 antibody is selected from the group consisting of: JSP191, CDX-0159, AB85, and FSI-174.

45. A pharmaceutical composition comprising the modified cells of any one of claims 25-44, and a pharmaceutically acceptable excipient, carrier, or diluent, optionally wherein the cells comprise HSCs and/or HSPCs.

46. The pharmaceutical composition of claim 45, further comprising an anti-CD117 antibody.

47. The pharmaceutical composition of claim 45 or claim 46, further comprising one or more anti-CD47, anti-CD40L, anti-CD122, anti-CD4, and/or anti-CD8 antibody.

48. A method of modifying a cell, optionally an HSC and/or HSPC, comprising introducing the nucleic acid of any one of claims 17-20 or the vector of any one of claims 21-24 into the cell, optionally wherein the cell is transiently or permanently modified, and optionally wherein the method is for preparing modified cells for hematopoietic cell transplantation (HCT) into a mammalian subject.

49. The method of claim 48, wherein the nucleic acid or vector is introduced into the cell by transfection, transduction, infection, electroporation, or nanopore technology.

50. The method of claim 48, wherein the nucleic acid is an mRNA introduced into the cell via electroporation.

51. A method of treating a mammalian subject in need thereof, comprising administering to the subject the pharmaceutical composition of any one of claims 45-47.

52. The method of claim 51, wherein the subject is administered a conditioning regimen to facilitate or increase engraftment of the modified cells, wherein the conditioning regimen is administered prior to and/or concurrent with and/or following the administering of the pharmaceutical composition.

53. The method of claim 52, wherein the conditioning regimen comprises administration of an anti-CD117 antibody to the subject.

54. The method of claim 53, wherein the anti-CD117 antibody is administered to the subject prior to administration of the pharmaceutical composition to the subject.

55. The method of claim 52, wherein the method comprises:
(i) selectively ablating endogenous hematopoietic stem cells in the subject by administering to the subject the anti-CD117 antibody;
(ii) optionally, waiting for a period of time following administration of the anti-CD117 antibody; and
(iii) following (ii), administering to the subject the pharmaceutical composition comprising the modified cells hematopoietic stem cells in a dose effective to achieve long term multilineage peripheral blood chimerism,
wherein the period of time is less than 5 days.

56. The method of claim 55, wherein the method comprises administering the anti-CD117 antibody and the pharmaceutical composition during an overlapping period of time or at about the same time.

57. The method of any one of claims 51-56, wherein the subject is treated for a disease or disorder selected from the group consisting of: a cancer, a cardiac disorder, a neural disorder, an autoimmune disease, an immunodeficiency, a metabolic disorder, and a genetic disorder.

58. The method of claim 57, wherein the cancer is a solid tissue cancer or a blood cancer.

59. The method of claim 58, wherein the blood cancer is a leukemia, a lymphoma, or a myelodysplastic syndrome.

60. The method of claim 59, wherein the leukemia is acute myeloid leukemia (AML).

61. The method of claim 57, wherein the immunodeficiency is severe combined immunodeficiency (SCID).

62. The method of claim 57, wherein the genetic disorder is sickle cell disease or Fanconi anemia.

63. The method of any one of claims 51-62, further comprising administering to the subject an additional therapeutic agent for treatment of the disease or disorder.


SEQUENCE LISTING (54 sequences)

SEQ ID NO: 1 - protein, 976 aa, Homo sapiens [sequence omitted]

SEQ ID NO: 2 - protein, 972 aa, Homo sapiens
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 3-4 - skipped (2 entries)

SEQ ID NO: 5 - DNA, 3132 bp, synthetic construct
  source 1..3132: mol_type = other DNA
taatacgactcactataaggactcttctggtccccacagactcagagagaacccaccgccgccaccatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctaatgagctcgctttcttgctgtccaatttctattaaaggttcctttgttccctaagtccaactactaaactgggggatattatgaagggccttgagcatctggattctgcctaataaaaaacatttattttcattgc

SEQ ID NO: 6 - DNA, 3132 bp, synthetic construct
  source 1..3132: mol_type = other DNA
taatacgactcactataaggactcttctggtccccacagactcagagagaacccaccgccgccaccatgagaggcgcccggggcgcatgggacttcttatgtgtattattgctcctcctgagggttcagaccgggagtagccaaccttccgtgtcccctggtgaaccatcgccaccatcgatccaccctggcaaatccgacctgatcgtgagggtcggcgatgaaatccggctcctatgcacggaccctggttttgtgaaatggacctttgagatcctcgatgaaaccaatgagaataaacagaacgagtggattacagaaaaggctgaggccacaaatacaggtaaatacacatgcaccaataagcacggcctctcgaactctatctacgtttttgtgcgagatcctgcaaaacttttcctcgtcgatcgctccttatacggaaaggaggacaatgatacactggtcagatgccctttaacagaccccgaggttacaaactattcactcaagggttgtcagggtaaacctctgccaaaagacctgcgctttatccctgatcctaaagccggcatcatgataaaatctgtaaagcgggcctaccaccgtctctgcttgcactgtagcgttgatcaagaaggcaagtcagttttaagcgagaaattcatcctgaaggtgcgcccagccttcaaggctgtgcctgtagtgtcagtgtctaaggcctcatacctgctccgggagggggaggaatttactgtgacctgcacgataaaagacgtttcctcttctgtgtactctacttggaagagggaaaatagccagaccaagctgcaggagaagtacaacagctggcatcatggtgacttcaactacgagagacaggccactctgactatttcatctgcacgcgtgaatgactccggtgtgtttatgtgctacgcaaataacaccttcggcagtgccaatgtgacaactaccctggaagtcgttgacaagggcttcattaacatctttccaatgatcaatacaaccgtctttgttaacgatggcgaaaacgtggacctgatcgttgaatatgaagcattccccaaaccagagcaccagcagtggatctacatgaatcgcactttcaccgacaagtgggaagactaccccaaatccgagaacgagtctaacatccgctatgtgtcggaactccacctgactagacttaagggtacggaaggaggcacctacacctttttggtgagcaatagcgacgtgaacgcggcaattgcttttaacgtatacgtgaatacgaaacccgaaatattgacatatgaccgtctggtgaacggaatgcttcagtgtgtggccgcaggctttcctgaaccaaccatcgactggtacttttgccctggtaccgagcagcggtgctccgcgagcgtgctgcctgtggacgtccagacgctaaattctagtgggccaccttttggaaaactggtggttcagtcgtcaattgattcttctgcatttaagcataatgggacagtggagtgtaaagcttacaacgatgtggggaagacaagcgcctatttcaactttgcctttaaggggaacaataaagagcagattcatccacacaccttgttcactcctttattgatcgggtttgtgatcgtggcgggaatgatgtgtattatcgttatgattttgacttataagtacctgcagaagcctatgtatgaagtgcagtggaaagtggtggaagagatcaacgggaacaattacgtttatatcgaccccacccagttgccatatgaccacaaatgggaatttcccaggaatcgcttgagcttcgggaagacactcggtgccggagccttcggaaaggtagtagaagcaacggcttacgggctaatcaagtcagatgccgctatgactgttgcggtgaaaatgttgaagccatcggctcatctgacagagcgggaggctctgatgagcgaactcaaggttctcagttacctcggcaatcacatgaacattgtaaatctccttggggcctgtacgatcggcggtcccaccctcgtcataacagaatactgctgctatggcgatctgctgaacttcctccggcgcaagagggattcctttatatgtagcaaacaagaagaccacgcggaggccgctctatacaaaaacctgttacacagtaaagagtcttcatgcagcgacagtacgaatgaatacatggacatgaaacctggggtaagttatgttgtgcctactaaagccgacaagcggcgcagcgtcaggatcggatcctatattgagagggacgtgacacctgctattatggaagatgacgagttagcattggacctcgaggaccttctatccttttcatatcaggtggccaagggcatggccttcctggcgtctaaaaactgtattcaccgcgatttggccgcgagaaacatcctgctcacacatggaagaatcaccaaaatttgcgactttggcctggccagagatatcaagaacgactcaaactatgtggtgaagggaaatgcacgtctgcccgtgaagtggatggcaccagagtcaatctttaattgtgtgtatacgttcgaaagtgatgtctggtcatacggaatcttcctgtgggagttgttctccctggggtcttccccctacccagggatgcctgtggactctaaattctacaagatgatcaaggagggcttccgcatgttatcaccagagcacgcacccgctgagatgtacgatattatgaaaacttgctgggacgctgatcccctaaagcggccaactttcaaacagattgttcagcttattgaaaagcagatcagtgaatctacaaaccatatctatagtaatctggccaattgctcacctaaccgacagaagcccgtggttgatcactccgttaggatcaactctgtgggcagcactgcaagctccagtcagcccctgcttgtccacgatgatgtctaatgagctcgctttcttgctgtccaatttctattaaaggttcctttgttccctaagtccaactactaaactgggggatattatgaagggccttgagcatctggattctgcctaataaaaaacatttattttcattgc

SEQ ID NO: 7 - DNA, 3132 bp, synthetic construct
  source 1..3132: mol_type = other DNA
taatacgactcactataaggactcttctggtccccacagactcagagagaacccaccgccgccaccatgagaggcgcccggggcgcatgggacttcttatgtgtattattgctcctcctgagggttcagaccgggagtagccaaccttccgtgtcccctggtgaaccatcgccaccatcgatccaccctggcaaatccgacctgatcgtgagggtcggcgatgaaatccggctcctatgcacggaccctggttttgtgaaatggacctttgagatcctcgatgccaccaatgagaataaacagaacgagtggattacagaaaaggctgaggccacaaatacaggtaaatacacatgcaccaataagcacggcctctcgaactctatctacgtttttgtgcgagatcctgcaaaacttttcctcgtcgatcgctccttatacggaaaggaggacaatgatacactggtcagatgccctttaacagaccccgaggttacaaactattcactcaagggttgtcagggtaaacctctgccaaaagacctgcgctttatccctgatcctaaagccggcatcatgataaaatctgtaaagcgggcctaccaccgtctctgcttgcactgtagcgttgatcaagaaggcaagtcagttttaagcgagaaattcatcctgaaggtgcgcccagccttcaaggctgtgcctgtagtgtcagtgtctaaggcctcatacctgctccgggagggggaggaatttactgtgacctgcacgataaaagacgtttcctcttctgtgtactctacttggaagagggaaaatagccagaccaagctgcaggagaagtacaacagctggcatcatggtgacttcaactacgagagacaggccactctgactatttcatctgcacgcgtgaatgactccggtgtgtttatgtgctacgcaaataacaccttcggcagtgccaatgtgacaactaccctggaagtcgttgacaagggcttcattaacatctttccaatgatcaatacaaccgtctttgttaacgatggcgaaaacgtggacctgatcgttgaatatgaagcattccccaaaccagagcaccagcagtggatctacatgaatcgcactttcaccgacaagtgggaagactaccccaaatccgagaacgagtctaacatccgctatgtgtcggaactccacctgactagacttaagggtacggaaggaggcacctacacctttttggtgagcaatagcgacgtgaacgcggcaattgcttttaacgtatacgtgaatacgaaacccgaaatattgacatatgaccgtctggtgaacggaatgcttcagtgtgtggccgcaggctttcctgaaccaaccatcgactggtacttttgccctggtaccgagcagcggtgctccgcgagcgtgctgcctgtggacgtccagacgctaaattctagtgggccaccttttggaaaactggtggttcagtcgtcaattgattcttctgcatttaagcataatgggacagtggagtgtaaagcttacaacgatgtggggaagacaagcgcctatttcaactttgcctttaaggggaacaataaagagcagattcatccacacaccttgttcactcctttattgatcgggtttgtgatcgtggcgggaatgatgtgtattatcgttatgattttgacttataagtacctgcagaagcctatgtatgaagtgcagtggaaagtggtggaagagatcaacgggaacaattacgtttatatcgaccccacccagttgccatatgaccacaaatgggaatttcccaggaatcgcttgagcttcgggaagacactcggtgccggagccttcggaaaggtagtagaagcaacggcttacgggctaatcaagtcagatgccgctatgactgttgcggtgaaaatgttgaagccatcggctcatctgacagagcgggaggctctgatgagcgaactcaaggttctcagttacctcggcaatcacatgaacattgtaaatctccttggggcctgtacgatcggcggtcccaccctcgtcataacagaatactgctgctatggcgatctgctgaacttcctccggcgcaagagggattcctttatatgtagcaaacaagaagaccacgcggaggccgctctatacaaaaacctgttacacagtaaagagtcttcatgcagcgacagtacgaatgaatacatggacatgaaacctggggtaagttatgttgtgcctactaaagccgacaagcggcgcagcgtcaggatcggatcctatattgagagggacgtgacacctgctattatggaagatgacgagttagcattggacctcgaggaccttctatccttttcatatcaggtggccaagggcatggccttcctggcgtctaaaaactgtattcaccgcgatttggccgcgagaaacatcctgctcacacatggaagaatcaccaaaatttgcgactttggcctggccagagatatcaagaacgactcaaactatgtggtgaagggaaatgcacgtctgcccgtgaagtggatggcaccagagtcaatctttaattgtgtgtatacgttcgaaagtgatgtctggtcatacggaatcttcctgtgggagttgttctccctggggtcttccccctacccagggatgcctgtggactctaaattctacaagatgatcaaggagggcttccgcatgttatcaccagagcacgcacccgctgagatgtacgatattatgaaaacttgctgggacgctgatcccctaaagcggccaactttcaaacagattgttcagcttattgaaaagcagatcagtgaatctacaaaccatatctatagtaatctggccaattgctcacctaaccgacagaagcccgtggttgatcactccgttaggatcaactctgtgggcagcactgcaagctccagtcagcccctgcttgtccacgatgatgtctaatgagctcgctttcttgctgtccaatttctattaaaggttcctttgttccctaagtccaactactaaactgggggatattatgaagggccttgagcatctggattctgcctaataaaaaacatttattttcattgc

SEQ ID NO: 8-13 - skipped (6 entries)

SEQ ID NO: 14 - protein, 271 aa, Homo sapiens
MKKTQTWILTCIYLQLLLFNPLVKTEGICRNRVTNNVKDVTKLVANLPKDYMITLKYVPGMDVLPSHCWISEMVVQLSDSLTDLLDKFSNISEGLSNYSIIDKLVNIVDDLVECVKENSSKDLKKSFKSPEPRLFTPEEFFRIFNRSIDAFKDFWASETSDCWSSTLSPEKDSRVSVTKPFMLPPVAASSLRNDSSSSNRKAKNPPGDSSLHWAAMALPALFSLIIGFAFGALYWKKRQPSLTRAVENIQINEEDNEISMLQEKEREFQEV

SEQ ID NO: 15 - DNA, 37 bp, synthetic construct
  source 1..37: mol_type = other DNA
actcttctggtccccacagactcagagagaacccacc

SEQ ID NO: 16 - DNA, 132 bp, synthetic construct
  source 1..132: mol_type = other DNA
gctcgctttcttgctgtccaatttctattaaaggttcctttgttccctaagtccaactactaaactgggggatattatgaagggccttgagcatctggattctgcctaataaaaaacatttattttcattgc

SEQ ID NO: 17 - skipped

SEQ ID NO: 18 - DNA, 5190 bp, Homo sapiens
  source 1..5190: mol_type = other DNA
tctgggggctcggctttgccgcgctcgctgcacttgggcgagagctggaacgtggaccagagctcggatcccatcgcagctaccgcgatgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaaggtaacaacaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctgagcagaatcagtgtttgggtcacccctccaggaatgatctcttcttttggcttccatgatggttattttcttttctttcaacttgcatccaactccaggatagtgggcaccccactgcaatcctgtctttctgagcacactttagtggccgatgatttttgtcatcagccaccatcctattgcaaaggttccaactgtatatattcccaatagcaacgtagcttctaccatgaacagaaaacattctgatttggaaaaagagagggaggtatggactgggggccagagtcctttccaaggcttctccaattctgcccaaaaatatggttgatagtttacctgaataaatggtagtaatcacagttggccttcagaaccatccatagtagtatgatgatacaagattagaagctgaaaacctaagtcctttatgtggaaaacagaacatcattagaacaaaggacagagtatgaacacctgggcttaagaaatctagtatttcatgctgggaatgagacataggccatgaaaaaaatgatccccaagtgtgaacaaaagatgctcttctgtggaccactgcatgagcttttatactaccgacctggtttttaaatagagtttgctattagagcattgaattggagagaaggcctccctagccagcacttgtatatacgcatctataaattgtccgtgttcatacatttgaggggaaaacaccataaggtttcgtttctgtatacaaccctggcattatgtccactgtgtatagaagtagattaagagccatataagtttgaaggaaacagttaataccattttttaaggaaacaatataaccacaaagcacagtttgaacaaaatctcctcttttagctgatgaacttattctgtagattctgtggaacaagcctatcagcttcagaatggcattgtactcaatggatttgatgctgtttgacaaagttactgattcactgcatggctcccacaggagtgggaaaacactgccatcttagtttggattcttatgtagcaggaaataaagtataggtttagcctccttcgcaggcatgtcctggacaccgggccagtatctatatatgtgtatgtacgtttgtatgtgtgtagacaaatatttggaggggtatttttgccctgagtccaagagggtcctttagtacctgaaaagtaacttggctttcattattagtactgctcttgtttcttttcacatagctgtctagagtagcttaccagaagcttccatagtggtgcagaggaagtggaaggcatcagtccctatgtatttgcagttcacctgcacttaaggcactctgttatttagactcatcttactgtacctgttccttagaccttccataatgctactgtctcactgaaacatttaaattttaccctttagactgtagcctggatattattcttgtagtttacctctttaaaaacaaaacaaaacaaaacaaaaaactccccttcctcactgcccaatataaaaggcaaatgtgtacatggcagagtttgtgtgttgtcttgaaagattcaggtatgttgcctttatggtttcccccttctacatttcttagactacatttagagaactgtggccgttatctggaagtaaccatttgcactggagttctatgctctcgcacctttccaaagttaacagattttggggttgtgttgtcacccaagagattgttgtttgccatactttgtctgaaaaattcctttgtgtttctattgacttcaatgatagtaagaaaagtggttgttagttatagatgtctaggtacttcaggggcacttcattgagagttttgtcttggatattcttgaaagtttatatttttataattttttcttacatcagatgtttctttgcagtggcttaatgtttgaaattattttgtggctttttttgtaaatattgaaatgtagcaataatgtcttttgaatattcccaagcccatgagtccttgaaaatattttttatatatacagtaactttatgtgtaaatacataagcggcgtaagtttaaaggatgttggtgttccacgtgttttattcctgtatgttgtccaattgttgacagttctgaagaattctaataaaatgtacatatataaatcaaaaaaaaaaaaaaaa

SEQ ID NO: 19 - protein, 499 aa, Homo sapiens
QPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTP

SEQ ID NO: 20 - protein, 466 aa, Homo sapiens
MDWTWRVFCLLAVAPGAHSQVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 238 aa, Homo sapiens
MVLQTQVFISLLLWISGAYGDIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 117 aa, Homo sapiens
QVQLVQSGAEVKKPGASVKVSCKASGYTFTSYNMHWVRQAPGQGLEWMGVIYSGNGDTSYNQKFKGRVTITADKSTSTAYMELSSLRSEDTAVYYCARERDTRFGNWGQGTLVTVSS

SEQ ID NO: 23 - protein, 111 aa, Homo sapiens
DIVMTQSPDSLAVSLGERATINCRASESVDIYGNSFMHWYQQKPGQPPKLLIYLASNLESGVPDRFSGSGSGTDFTLTISSLQAEDVAVYYCQQNNEDPYTFGGGTKVEIK

SEQ ID NO: 24 - protein, 4 aa, Homo sapiens
YNMH

SEQ ID NO: 25 - protein, 16 aa, Homo sapiens
IYSGNGDTSYNQKFKG

SEQ ID NO: 26 - protein, 8 aa, Homo sapiens
ERDTRFGN

SEQ ID NO: 27 - protein, 15 aa, Homo sapiens
RASESVDIYGNSFMH

SEQ ID NO: 28 - protein, 7 aa, Homo sapiens
LASNLES

SEQ ID NO: 29 - protein, 9 aa, Homo sapiens
QQNNEDPYT

SEQ ID NO: 30 - protein, 107 aa, synthetic construct
  VARIANT 10: note = X is an amino acid with an aromatic or aliphatic hydroxyl side chain
  VARIANT 46: note = X is an amino acid with an aliphatic or aliphatic hydroxyl side chain
  VARIANT 63: note = X is an amino acid with an aliphatic hydroxyl side chain
  VARIANT 80: note = X is an amino acid with an aliphatic hydroxyl side chain or is P
  VARIANT 85: note = X is an amino acid with a charged or acidic side chain
  VARIANT 87: note = X is an amino acid with an aromatic side chain
DIVMTQSPSXLSASVGDRVTITCKASQNVRTNVAWYQQKPGKAPKXLIYSASYRYSGVPDRFXGSGSGTDFTLTISSLQXEDFAXYXCQQYNSYPRTFGGGTKVEIK

SEQ ID NO: 31 - protein, 117 aa, synthetic construct
  VARIANT 11: note = X is an amino acid with an aliphatic side chain
  VARIANT 20: note = X is an amino acid with an aliphatic side chain
  VARIANT 39: note = X is an amino acid with a polar or basic side chain
  VARIANT 69: note = X is an amino acid with an aliphatic side chain
  VARIANT 71: note = X is an amino acid with an aliphatic side chain
  VARIANT 74: note = X is an amino acid with an acidic side chain
  VARIANT 83: note = X is an amino acid with an acidic or amide derivative side chain
  VARIANT 92: note = X is an amino acid with an aliphatic hydroxyl side chain
QVQLVQSGAEXKKPGASVKXSCKASGYTFTDYYINAVVXQAPGKGLEWIARIYPGSGNTYYNEKFKGRXTXTAXKSTSTAYMXLSSLRSEDXAVYFCARGVYYFDYWGQGTTVTVSS

SEQ ID NO: 32 - protein, 116 aa, synthetic construct
QVQLKQSGAELVRPGASVKLSCKASGYTFTDYYINWVKQRPGQGLEWIARIYPGSGNTYYNEKFKGKATLTAEKSSSTAYMQLSSLTSEDSAVYFCARGVYYFDYWGQGTTLTVSS

SEQ ID NO: 33 - protein, 116 aa, synthetic construct
QVQLKQSGAELVRPGASVKLSCKASGYTFTDYYINWVKQRPGQGLEWIARIYPGSGNTYYNEKFKGKATLTAEKSSSTAYMQLSSLTSEDSAVYFCARGVYYFDYWGQGTTLTVSA

SEQ ID NO: 34 - protein, 108 aa, synthetic construct
DIVMTQSQKFMSTSVGDRVSVTCKASQNVRTNVAWYQQKPGQSPKALIYSASYRYSGVPDRFTGSGSGTDFTLTISNVQSEDLADYFCQQYNSYPRTFGGGTKLEIKR

SEQ ID NO: 35 - protein, 31 aa, Homo sapiens
EKAEATNTGKYTCTNKHGLSNSIYVFVRDPA

SEQ ID NO: 36 - protein, 21 aa, Homo sapiens
RCPLTDPEVTNYSLKGCQGKP

SEQ ID NO: 37 - protein, 123 aa, synthetic construct
EVQLVQSGAEVKKPGESLKISCKGSGYSFTNYWIGWVRQMPGKGLEWMAIINPRDSDTRYRPSFQGQVTISADKSISTAYLQWSSLKASDTAMYYCARHGRGYEGYEGAFDIWGQGTLVTVSS

SEQ ID NO: 38 - protein, 5 aa, synthetic construct
NYWIG

SEQ ID NO: 39 - protein, 17 aa, synthetic construct
IINPRDSDTRYRPSFQG

SEQ ID NO: 40 - protein, 14 aa, synthetic construct
HGRGYEGYEGAFDI

SEQ ID NO: 41 - protein, 107 aa, synthetic construct
DIQMTQSPSSLSASVGDRVTITCRSSQGIRSDLGWYQQKPGKAPKLLIYDASNLETGVPSRFSGSGSGTDFTLTISSLQPEDFATYYCQQANGFPLTFGGGTKVEIK

SEQ ID NO: 42 - protein, 11 aa, synthetic construct
RSSQGIRSDLG

SEQ ID NO: 43 - protein, 7 aa, synthetic construct
DASNLET

SEQ ID NO: 44 - protein, 9 aa, synthetic construct
QQANGFPLT

SEQ ID NO: 45 - protein, 5 aa, synthetic construct
SYNMH

SEQ ID NO: 46 - protein, 17 aa, synthetic construct
  VARIANT 12: note = A or N
  VARIANT 16: note = K or Q
VIYSGNGDTSYXQKFXG

SEQ ID NO: 47 - protein, 15 aa, synthetic construct
  VARIANT 4: note = D or E
  VARIANT 11: note = N or Q
RASXSVDIYGXSFMH

SEQ ID NO: 48 - protein, 17 aa, synthetic construct
VIYSGNGDTSYAQKFKG

SEQ ID NO: 49 - protein, 15 aa, synthetic construct
RASESVDIYGQSFMH

SEQ ID NO: 50 - protein, 117 aa, synthetic construct
QVQLVQSGAEVKKPGASVKVSCKASGYTFTSYMNHWVRQAPGQGLEWMGVIYSGNGDTSYAQKFKGRVTITADKSTSTAYMELSSLRSEDTAVYYCARERDTRFGNWGQGTLVTVSS

SEQ ID NO: 51 - protein, 117 aa, synthetic construct
QVQLVQSGAEVKKPGASVKVSCKASGYTFTSYMNHWVRQAPGQGLEWMGVIYSGNGDTSYNQKFQGRVTITADKSTSTAYMELSSLRSEDTAVYYCARERDTRFGNWGQGTLVTVSS

SEQ ID NO: 52 - protein, 117 aa, synthetic construct
QVQLVQSGAEVKKPGASVKVSCKASGYTFTSYMNHWVRQAPGQGLEWMGVIYSGNGDTSYAQKFQGRVTITADKSTSTAYMELSSLRSEDTAVYYCARERDTRFGNWGQGTLVTVSS

SEQ ID NO: 53 - protein, 111 aa, synthetic construct
DIVMTQSPLSLPVTPGEPASISCRASESVDIYGQSFMHWYQQKPGQPPKLLIYLASNLESGVPDRFSGSGSGTDFTLKISRVEAEDVGVYYCQQNNEDPYTFGGGTKVEIK

SEQ ID NO: 54 - DNA, 2919 bp, synthetic construct
  source 1..2919: mol_type = other DNA
atgagaggcgctcgcggcgcctgggattttctctgcgttctgctcctactgcttcgcgtccagacaggctcttctcaaccatctgtgagtccaggggaaccgtctccaccatccatccatccaggaaaatcagacttaatagtccgcgtgggcgacgagattaggctgttatgcactgatccgggctttgtcaaatggacttttgagatcctggatgaaacgaatgagaataagcagaatgaatggatcacggaaaaggcagaagccaccaacaccggcaaatacacgtgcaccaacaaacacggcttaagcaattccatttatgtgtttgttagagatcctgccaagcttttccttgttgaccgctccttgtatgggaaagaagacaacgacacgctggtccgctgtcctctcacagacccagaagtgaccaattattccctcaaggggtgccaggggaagcctcttcccaaggacttgaggtttattcctgaccccaaggcgggcatcatgatcaaaagtgtgaaacgcgcctaccatcggctctgtctgcattgttctgtggaccaggagggcaagtcagtgctgtcggaaaaattcatcctgaaagtgaggccagccttcaaagctgtgcctgttgtgtctgtgtccaaagcaagctatcttcttagggaaggggaagaattcacagtgacgtgcacaataaaagatgtgtctagttctgtgtactcaacgtggaaaagagaaaacagtcagactaaactacaggagaaatataatagctggcatcacggtgacttcaattatgaacgtcaggcaacgttgactatcagttcagcgagagttaatgattctggagtgttcatgtgttatgccaataatacttttggatcagcaaatgtcacaacaaccttggaagtagtagataaaggattcattaatatcttccccatgataaacactacagtatttgtaaacgatggagaaaatgtagatttgattgttgaatatgaagcattccccaaacctgaacaccagcagtggatctatatgaacagaaccttcactgataaatgggaagattatcccaagtctgagaatgaaagtaatatcagatacgtaagtgaacttcatctaacgagattaaaaggcaccgaaggaggcacttacacattcctagtgtccaattctgacgtcaatgctgccatagcatttaatgtttatgtgaatacaaaaccagaaatcctgacttacgacaggctcgtgaatggcatgctccaatgtgtggcagcaggattcccagagcccacaatagattggtatttttgtccaggaactgagcagagatgctctgcttctgtactgccagtggatgtgcagacactaaactcatctgggccaccgtttggaaagctagtggttcagagttctatagattctagtgcattcaagcacaatggcacggttgaatgtaaggcttacaacgatgtgggcaagacttctgcctattttaactttgcatttaaagagcaaatccatccccacaccctgttcactcctttgctgattggtttcgtaatcgtagctggcatgatgtgcattattgtgatgattctgacctacaaatatttacagaaacccatgtatgaagtacagtggaaggttgttgaggagataaatggaaacaattatgtttacatagacccaacacaacttccttatgatcacaaatgggagtttcccagaaacaggctgagttttgggaaaaccctgggtgctggagctttcgggaaggttgttgaggcaactgcttatggcttaattaagtcagatgcggccatgactgtcgctgtaaagatgctcaagccgagtgcccatttgacagaacgggaagccctcatgtctgaactcaaagtcctgagttaccttggtaatcacatgaatattgtgaatctacttggagcctgcaccattggagggcccaccctggtcattacagaatattgttgctatggtgatcttttgaattttttgagaagaaaacgtgattcatttatttgttcaaagcaggaagatcatgcagaagctgcactttataagaatcttctgcattcaaaggagtcttcctgcagcgatagtactaatgagtacatggacatgaaacctggagtttcttatgttgtcccaaccaaggccgacaaaaggagatctgtgagaataggctcatacatagaaagagatgtgactcccgccatcatggaggatgacgagttggccctagacttagaagacttgctgagcttttcttaccaggtggcaaagggcatggctttcctcgcctccaagaattgtattcacagagacttggcagccagaaatatcctccttactcatggtcggatcacaaagatttgtgattttggtctagccagagacatcaagaatgattctaattatgtggttaaaggaaacgctcgactacctgtgaagtggatggcacctgaaagcattttcaactgtgtatacacgtttgaaagtgacgtctggtcctatgggatttttctttgggagctgttctctttaggaagcagcccctatcctggaatgccggtcgattctaagttctacaagatgatcaaggaaggcttccggatgctcagccctgaacacgcacctgctgaaatgtatgacataatgaagacttgctgggatgcagatcccctaaaaagaccaacattcaagcaaattgttcagctaattgagaagcagatttcagagagcaccaatcatatttactccaacttagcaaactgcagccccaaccgacagaagcccgtggtagaccattctgtgcggatcaattctgtcggcagcaccgcttcctcctcccagcctctgcttgtgcacgacgatgtctga